(19) United States
(12) Patent Application Publication
(10) Pub. No.: US 2010/0216821 A1
(43) Pub. Date: Aug. 26, 2010

(54) Benzimidazoles With A Hetero Spiro-Decane Residue As NPY-Y5 Antagonists

(76) Inventors: Nicholas Paul Barton, Essex (GB); Benjamin Richard Bellenie, Essex (GB); Jonathan Bentley, Verona (IT); Matteo Biagetti, Verona (IT); Thorsten Genski, Verona (IT); Colin Philip Leslie, Verona (IT); Angelica Mazzali, Verona (IT); Domenica Antonia Pizzi, Verona (IT); Fabio Maria Sabbatini, Verona (IT); Catia Seri, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/596,931

(22) PCT Filed: Apr. 18, 2008
(86) PCT No.: PCT/EP2008/054757

§ 371 Date: May 13, 2010

(30) Foreign Application Priority Data
Apr. 24, 2007 (GB) 0707934.6

(51) Int. Cl. A61K 31/52; A61P 3/04; A61P 25/24; C07D 498/10; A61K 31/438; C07D 471/10; C07D 413/08; A61K 31/423; C07D 403/14; A61K 31/506

(52) U.S. Cl. 514/263.2; 546/19; 514/278; 546/20; 548/216; 514/376; 544/230; 514/275

(57) ABSTRACT

The present invention relates to compounds of formula (I), or a pharmaceutically acceptable salts, solvates, stereoisomers thereof,

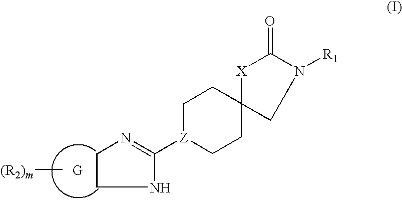

R₁ may be C₁-C₄ alkyl, aryl or heteroaryl, which may be substituted by one or more: halogen, C₁-C₄ alkyl, C₁-C₄ alkoxy, C₁-C₄ haloalkyl, C₁-C₄ haloalkoxy, cyano;
R₂ may be halogen, C₁-C₄ alkyl, C₁-C₄ alkoxy, C₁-C₄ haloalkyl, C₁-C₄ haloalkoxy, cyano, nitro; or aryl, heteroaryl or heterocycle, which may be substituted by one or more: halogen, C₁-C₄ alkyl, C₁-C₄ alkoxy, C₁-C₄ haloalkyl, C₁-C₄ haloalkoxy, cyano; or R₂ may correspond to —O—R₃;
R₃ is a 6-membered aromatic carbocyclic ring which may contain 1 or 2 nitrogen
X is carbon or oxygen;
Z is carbon or nitrogen;
G is a fused 6-membered aromatic carbocyclic ring which may contain 1 or 2 nitrogen;
m may be 0 or an integer ranging from 1 to 4;
   processes for their preparation, intermediates used in these processes, pharmaceutical compositions containing them and their use in therapy, as NPY-Y5 receptor antagonists.

The present invention relates to novel compounds, processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

Neuropeptide Y (hereinafter referred to as NPY), a peptide consisting of 36 amino acids, was first isolated from porcine brain by Tatemoto et al. in 1982 [Nature, 296: 659 (1982)]. NPY is widely distributed in central and peripheral nervous systems and plays various roles as one of the most abundant peptides in the nervous system. NPY acts as an orexigenic substance in the central nervous system and markedly promotes fat accumulation via the mediation of the secretion of various hormones or the action of the nervous system. It is known that the continuous intracerebroventricular administration of NPY induces obesity and insulin resistance based on these actions (International Journal of Obesity, vol. 19: 517 (1995); Endocrinology, vol. 133: 1753 (1993)). It is also known that NPY has central effects that are related to diseases such as depression, anxiety, schizophrenia, pain, dementia and the like (Drugs, vol. 52, 371 (1996). Furthermore, in the periphery, NPY coexists with norepinephrine in sympathetic nerve endings and is involved in the tonicity of the sympathetic nervous system. It is known that peripheral administration of NPY causes vasoconstriction and enhances the activities of other vasoconstrictive substances such as norepinephrine (British Journal of Pharmacology, vol. 95: 419 (1988)). It is also reported that NPY could participate in the development of cardiac hypertrophy as a result of the sympathetic stimulation (Proceeding National Academic Science USA, Vol. 97, 1595 (2000)).

Endogenous receptor proteins that bind NPY and related peptides as ligands have been identified and distinguished, and several such proteins have been cloned and expressed. Six different receptor subtypes [Y1, Y2, Y3, Y4 (PP), Y5, Y6] are recognised today based upon binding profile, pharmacology and/or composition if identity is known.

The Y5 subtype was isolated, characterized and reported recently in U.S. Pat. No. 5,602,024 (WO 96/16542). The effects mediated by the NPY Y5 receptor include eating stimulation and accumulation of fat (Nature, vol. 382, 168 (1996)); American Journal of Physiology, vol. 277, R1428 (1999)). It is reported that the NPY Y5 receptor also mediates some CNS effects, such as seizure and epilepsy, or pain and morphine withdrawal symptoms (Natural Medicine, vol. 3, 761 (1997); Proceeding Academic Science USA, vol. 96, 13518 (1999); The Journal of Pharmacology and Experimental Therapetics, vol. 284, 633 (1998)). In the periphery, the NPY Y5 receptor is reported to be involved in diuresis and the hypoglycemic effect caused by NPY (British Journal of Pharmacology, vol. 120, 1335 (1998); Endocrinology, vol. 139, 3018 (1998)). NPY is also reported to enhance cardiac hypertrophy as a result of sympathetic accentuation (Proceeding National Academic Science USA, Vol. 97, 1595 (2000)).

The effects of NPY occur by binding to the NPY receptors in the central or peripheral nervous system. Therefore, the action of NPY can be prevented by blocking the binding to NPY receptors. Substances that antagonize NPY binding to NPY receptors may be useful for the prophylaxis or treatment of various diseases related to NPY, such as cardiovascular disorders (for example hypertension, nephropathy, heart disease, vasospasm), central nervous system disorders (for example bulimia, binge eating, depression, anxiety, seizure, epilepsy, dementia, pain, alcoholism, drug withdrawal), metabolic diseases (for example obesity, diabetes, hormone abnormality), sexual and reproductive dysfunction, gastro-intestinal motility disorder, respiratory disorder, inflammation or glaucoma and the like (Trends in Pharmacological Sciences, 15: 153 (1994); Life Science, 55, 551 (1994); Drugs, vol. 52, 371 (1996); The Journal of Allergy and Immunology, vol. 101, S345 (1998); Nature, vol. 396, 366 (1998); The Journal of Pharmacology and Experimental Therapeutics, vol. 284, 633 (1998); Trends in Pharmacological Science, vol. 20, 104 (1999); Proceeding National Academic Science USA, vol. 97, 1595 (2000)).

The object of the present invention is to provide compounds of formula (I) or a pharmaceutically acceptable salt or solvate thereof:

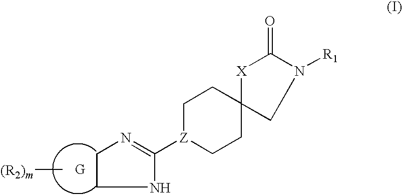

R₁ may be C₁-C₄ alkyl, aryl or heteroaryl, which may be substituted by one or more: halogen, C₁-C₄ alkyl, C₁-C₄ alkoxy, C₁-C₄ haloalkyl, C₁-C₄ haloalkoxy, cyano;
R₂ may be halogen, C₁-C₄ alkyl, C₁-C₄ alkoxy, C₁-C₄ haloalkyl, C₁-C₄ haloalkoxy, cyano, nitro; or aryl, heteroaryl or heterocycle, which may be substituted by one or more: halogen, C₁-C₄ alkyl, C₁-C₄ alkoxy, C₁-C₄ haloalkyl, C₁-C₄ haloalkoxy, cyano; or R₂ may correspond to —O—R₃;
R₃ is a 6-membered aromatic carbocyclic ring which may contain 1 or 2 nitrogen
X is carbon or oxygen;
Z is carbon or nitrogen;
G is a fused 6-membered aromatic carbocyclic ring which may contain 1 or 2 nitrogen;
m may be 0 or an integer ranging from 1 to 4.

The compounds of the present invention may be in the form of and/or may be administered as a pharmaceutically acceptable salt. For a review on suitable salts see Berge et al, J. Pharm. Sci., 1977, 66, 1-19.

Typically, a pharmaceutically acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, pyruvate, oxalate, oxaloacetate, trifluoroacetate, saccharate, benzoate, methanesulphonate, ethanesulphonate, benzenesulphonate, p-toluenesulphonate and isethionate.

Pharmaceutically acceptable base salts include ammonium salts, alkali metal salts such as those of sodium and potassium, alkaline earth metal salts such as those of calcium and magnesium.

Pharmaceutically acceptable salts may also be prepared from other salts, including other pharmaceutically acceptable salts, of the compound of formula (I) using conventional methods.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

With regard to stereoisomers, the compounds of general formula (I) may have one or more asymmetric carbon atoms and may occur as racemates, racemic mixtures and as individual enantiomers or diastereomers. All such isomeric forms are included within the present invention, including mixtures thereof.

When a specific enantiomer of a compound of general formula (I) is required, this may be obtained for example by resolution of a corresponding enantiomeric mixture of a compound of formula (I) using conventional methods, such as H.P.L.C. of the corresponding racemate using a suitable chiral support or by fractional crystallisation of the diastereoisomeric salts formed by reaction of the corresponding racemate with a suitable optically active acid or base, as appropriate.

Or a specific enantiomer may also be prepared from a corresponding optically pure intermediate.

Separation of diastereoisomers or cis and trans isomers or syn and anti isomers may be achieved by conventional techniques, e.g. by fractional crystallisation, chromatography or H.P.L.C. of a stereoisomeric mixture.

Furthermore, some of the crystalline forms of the compounds of structure (I) may exist as polymorphs, which are included in the present invention.

The term C₁-C₄ alkyl as used herein as a group or a part of the group refers to a linear or branched alkyl group containing from 1 to 4 carbon atoms; examples of such groups include methyl, ethyl, propyl, isopropyl, n-butyl, isobutyl, tert butyl.

The term halogen refers to a fluorine, chlorine, bromine or iodine atom.

The term halo C₁-C₄ alkyl means an alkyl group having one to 4 carbon atoms and wherein at least one hydrogen atom is replaced with halogen such as for example a trifluoromethyl group and the like.

The term C₁-C₄ alkoxy group may be a linear or a branched chain alkoxy group, for example methoxy, ethoxy, propoxy, prop-2-oxy, butoxy, but-2-oxy or methylprop-2-oxy and the like.

The term halo C₁-C₄ alkoxy group may be a C₁-C₄ alkoxy group as defined before substituted with at least one halogen, preferably fluorine, such as OCHF₂, or OCF₃.

The term aryl means an aromatic carbocyclic moiety such as phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono- and bicyclic ring systems.

Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, and benzodioxolyl.

The term heterocycle means a 5 to 7-membered monocyclic, or 7- to 14-membered polycyclic, heterocycle ring which is either saturated or unsaturated, and which contains from 1 to 4 heteroatoms independently selected from nitrogen, oxygen and sulfur, and wherein the heterocycle may contain an amide or urea moiety. The heterocycle may be attached via any heteroatom or carbon atom. Heterocycles include (but are not limited to) 2,3-dihydro-1,4-benzodioxinyl, 2(1H)-pyridinonyl and 3(2H)-pyridazinonyl 1-methyl-2-imidazolidinonyl and the like.

The 6-membered aromatic carbocyclic rings which may contain 1 or 2 nitrogen include phenyl, pyrimidinyl, pyridinyl, pyrazinyl and pyridazinyl.

With regard to stereoisomers, the compounds of general formula (I)′, which correspond to the compounds of formula (I) when Z is carbon

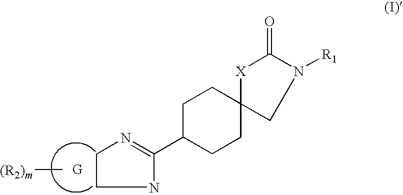

can exist as two stereoisomers represented by the general formulas (Ia)′ and (Ib)′.

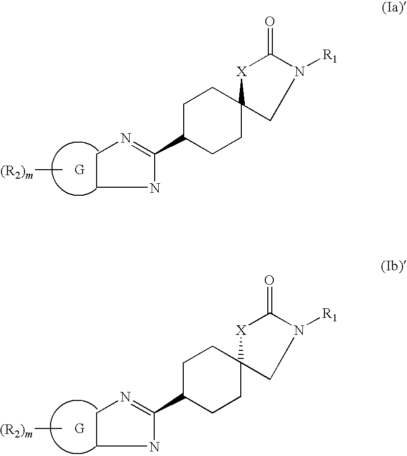

In one embodiment compounds of formula (Ia)′ are provided in which the stereochemistry is “cis”. In another embodiment of the present invention, compounds of formula (Ib)′ are provided and in which the stereochemistry is “trans”.

“Trans” stereochemistry is due to highest priority groups, according to Kahn-Prelog-Ingold classification, attached to the cyclohexane ring being on opposite sides of the cyclohexane ring. “Trans” stereochemistry can be designated also as “trans configuration” or “anti”; in the case of formula (Ib)′ description (5r,8r) can also be used to describe the “trans” stereochemistry.

In one embodiment, X is carbon or oxygen. In another embodiment, X is oxygen.

In one embodiment, Z is carbon or nitrogen. In another embodiment, Z is carbon.

In one embodiment, R₁ is C₁-C₄ alkyl. In another embodiment, R₁ is aryl or heteroaryl, which may be substituted by one or more: halogen, C₁-C₄ alkyl, C₁-C₄ alkoxy, C₁-C₄ haloalkyl, C₁-C₄ haloalkoxy, cyano.

In one embodiment, G is a phenyl ring.

In one embodiment, m is 0, 1 or 2. In another embodiment, m is 1.

In one embodiment, R₂ is halogen, C₁-C₄ alkyl, C₁-C₄ alkoxy, C₁-C₄ haloalkyl, C₁-C₄ haloalkoxy, cyano, nitro; or is aryl, heteroaryl or heterocycle, which may be substituted by one or more: halogen, C₁-C₄ alkyl, C₁-C₄ alkoxy, C₁-C₄ haloalkyl, C₁-C₄ haloalkoxy, cyano; or R₂ is —O—R₃. In another embodiment, R₂ is halogen, C₁-C₄ alkyl, C₁-C₄ alkoxy, C₁-C₄ haloalkyl, C₁-C₄ haloalkoxy, cyano, nitro. In a further embodiment, R₂ is aryl, heteroaryl or heterocycle, which may be substituted by one or more: halogen, C₁-C₄ alkyl, C₁-C₄ alkoxy, C₁-C₄ haloalkyl, C₁-C₄ haloalkoxy, cyano. In a still further embodiment, R₂ is —O—R₃.

In one embodiment of the present invention, compounds of formula (Ib)′ are provided, corresponding to compounds of formula (I), in which R₁ is aryl or heteroaryl and may be substituted by one or more: halogen, C₁-C₄ alkyl, C₁-C₄ alkoxy, C₁-C₄ haloalkyl, C₁-C₄ haloalkoxy, cyano; and R₂, X, and G are defined as above.

In another embodiment of the present invention, compounds of formula (Ib)′ are provided, corresponding to compounds of formula (I), in which R₁ is phenyl or 2-pyridine and may be substituted by one or more: halogen, C₁-C₄ alkyl, C₁-C₄ alkoxy, C₁-C₄ haloalkyl, C₁-C₄ haloalkoxy, cyano; X is oxygen, g is phenyl and R₂ is as above defined.

Example compounds of the present invention include:
8-(1H-benzimidazol-2-yl)-3-phenyl-1-oxa-3,8-diazaspiro[4.5]decan-2-one);
3-phenyl-8-[5-(trifluoromethyl)-1H-benzimidazol-2-yl]-1-oxa-3,8-diazaspiro[4.5]decan-2-one;
(cis)-3-phenyl-8-[5-(trifluoromethyl)-1H-benzimidazol-2-yl]-1-oxa-3-azaspiro[4.5]decan-2-one;
(trans)-3-phenyl-8-[5-(trifluoromethyl)-1H-benzimidazol-2-yl]-1-oxa-3-azaspiro[4.5]decan-2-one;
8-(5-bromo-1H-benzimidazol-2-yl)-3-phenyl-1-oxa-3,8-diazaspiro[4.5]decan-2-one;
8-(5,6-dichloro-1H-benzimidazol-2-yl)-3-phenyl-1-oxa-3,8-diazaspiro[4.5]decan-2-one;
8-(5-fluoro-1H-benzimidazol-2-yl)-3-phenyl-1-oxa-3,8-diazaspiro[4.5]decan-2-one;
3-(3,4-dichlorophenyl)-8-[5-(trifluoromethyl)-1H-benzimidazol-2-yl]-1-oxa-3,8-diazaspiro[4.5]decan-2-one;
8-[5-(4-fluorophenyl)-1H-benzimidazol-2-yl]-3-phenyl-1-oxa-3,8-diazaspiro[4.5]decan-2-one;
8-(5-chloro-1H-benzimidazol-2-yl)-3-phenyl-1-oxa-3,8-diazaspiro[4.5]decan-2-one;
8-[5-(methyloxy)-1H-benzimidazol-2-yl]-3-phenyl-1-oxa-3,8-diazaspiro[4.5]decan-2-one;
3-methyl-8-[5-(trifluoromethyl)-1H-benzimidazol-2-yl]-1-oxa-3,8-diazaspiro[4.5]decan-2-one;
8-(5-chloro-1H-benzimidazol-2-yl)-3-methyl-1-oxa-3,8-diazaspiro[4.5]decan-2-one;
3-methyl-8-[5-(methyloxy)-1H-benzimidazol-2-yl]-1-oxa-3,8-diazaspiro[4.5]decan-2-one;
3-phenyl-8-{5-[(trifluoromethyl)oxy]-1H-benzimidazol-2-yl}-1-oxa-3,8-diazaspiro[4.5]decan-2-one;
(trans)-2-phenyl-8-[5-(trifluoromethyl)-1H-benzimidazol-2-yl]-2-azaspiro[4.5]decan-3-one;
(cis)-2-phenyl-8-[5-(trifluoromethyl)-1H-benzimidazol-2-yl]-2-azaspiro[4.5]decan-3-one;
(cis)-3-phenyl-8-{5-[(trifluoromethyl)oxy]-1H-benzimidazol-2-yl}-1-oxa-3-azaspiro[4.5]decan-2-one;
(trans)-3-phenyl-8-{5-[(trifluoromethyl)oxy]-1H-benzimidazol-2-yl}-1-oxa-3-azaspiro[4.5]decan-2-one;
(trans)-3-(2-fluorophenyl)-8-{5-[(trifluoromethyl)oxy]-1H-benzimidazol-2-yl}-1-oxa-3-azaspiro[4.5]decan-2-one;
(trans)3-(4-fluorophenyl)-8-{5-[(trifluoromethyl)oxy]-1H-benzimidazol-2-yl}-1-oxa-3-azaspiro[4.5]decan-2-one;
2-[(trans)-2-oxo-3-(2-pyridinyl)-1-oxa-3-azaspiro[4.5]dec-8-yl]-1H-benzimidazole-5-carbonitrile;
(trans)-3-(2-pyridinyl)-8-{5-[(trifluoromethyl)oxy]-1H-benzimidazol-2-yl}-1-oxa-3-azaspiro[4.5]decan-2-one;
2-[(trans)-2-oxo-3-phenyl-1-oxa-3-azaspiro[4.5]dec-8-yl]-1H-benzimidazole-5-carbonitrile;
2-[(trans)-3-(4-fluorophenyl)-2-oxo-1-oxa-3-azaspiro[4.5]dec-8-yl]-1H-benzimidazole-5-carbonitrile;
(trans)-3-(2-pyridinyl)-8-[5-(trifluoromethyl)-1H-benzimidazol-2-yl]-1-oxa-3-azaspiro[4.5]decan-2-one;
(trans)-3-(6-methyl-2-pyridinyl)-8-{5-[(trifluoromethyl)oxy]-1H-benzimidazol-2-yl}-1-oxa-3-azaspiro[4.5]decan-2-one;
(trans)-3-(2-methyl-3-pyridinyl)-8-{5-[(trifluoromethyl)oxy]-1H-benzimidazol-2-yl}-1-oxa-3-azaspiro[4.5]decan-2-one;
(trans)-3-(2-pyrimidinyl)-8-{5-[(trifluoromethyl)oxy]-1H-benzimidazol-2-yl}-1-oxa-3-azaspiro[4.5]decan-2-one;
(trans)-3-(2-fluorophenyl)-8-[5-(1H-pyrazol-1-yl)-1H-benzimidazol-2-yl]-1-oxa-3-azaspiro[4.5]decan-2-one;
(trans)-3-(2-fluorophenyl)-8-[5-(3-methyl-2-oxo-1-imidazolidinyl)-1H-benzimidazol-2-yl]-1-oxa-3-azaspiro[4.5]decan-2-one;
(trans)-3-(2-fluorophenyl)-8-[5-(1H-imidazol-1-yl)-1H-benzimidazol-2-yl]-1-oxa-3-azaspiro[4.5]decan-2-one;
(trans)-3-(2-fluorophenyl)-8-[5-(2-oxo-1(2H)-pyridinyl)-1H-benzimidazol-2-yl]-1-oxa-3-azaspiro[4.5]decan-2-one;
(trans)-3-(2-fluorophenyl)-8-[5-(6-oxo-1(6H)-pyridazinyl)-1H-benzimidazol-2-yl]-1-oxa-3-azaspiro[4.5]decan-2-one;
(trans)-3-(2-pyridinyl)-8-[5-(3-pyridinyloxy)-1H-benzimidazol-2-yl]-1-oxa-3-azaspiro[4.5]decan-2-one;
(trans)-3-(3-pyridinyl)-8-{5-[(trifluoromethyl)oxy]-1H-benzimidazol-2-yl}-1-oxa-3-azaspiro[4.5]decan-2-one;
(trans)-3-(2-fluoro-3-pyridinyl)-8-{5-[(trifluoromethyl)oxy]-1H-benzimidazol-2-yl}-1-oxa-3-azaspiro[4.5]decan-2-one;
(trans)-3-(2-methylphenyl)-8-{5-[(trifluoromethyl)oxy]-1H-benzimidazol-2-yl}-1-oxa-3-azaspiro[4.5]decan-2-one;
(trans)-3-(2-chlorophenyl)-8-{5-[(trifluoromethyl)oxy]-1H-benzimidazol-2-yl}-1-oxa-3-azaspiro[4.5]decan-2-one;
(trans)-8-(5-bromo-1H-benzimidazol-2-yl)-3-(2-pyridinyl)-1-oxa-3-azaspiro[4.5]decan-2-one;
(trans)-8-[5-(3,5-dimethyl-4-isoxazolyl)-1H-benzimidazol-2-yl]-3-(2-pyridinyl)-1-oxa-3-azaspiro[4.5]decan-2-one;
(trans)-8-(5-bromo-1H-benzimidazol-2-yl)-3-(2-fluorophenyl)-1-oxa-3-azaspiro[4.5]decan-2-one;
(trans)-8-[5-(2,3-dihydro-1,4-benzodioxin-6-yl)-1H-benzimidazol-2-yl]-3-(2-fluorophenyl)-1-oxa-3-azaspiro[4.5]decan-2-one;
(trans)-3-(2-fluorophenyl)-8-{5-[2-(methyloxy)-5-pyrimidinyl]-1H-benzimidazol-2-yl}-1-oxa-3-azaspiro[4.5]decan-2-one;
(trans)-8-(1H-benzimidazol-2-yl)-3-(2-fluorophenyl)-1-oxa-3-azaspiro[4.5]decan-2-one;
3-(2-fluorophenyl)-8-[5-(4-pyridazinyl)-1H-benzimidazol-2-yl]-1-oxa-3-azaspiro[4.5]decan-2-one;
(trans)-3-(2-fluorophenyl)-8-[5-(5-pyrimidinyl)-1H-benzimidazol-2-yl]-1-oxa-3-azaspiro[4.5]decan-2-one;
(trans)-3-(2-fluorophenyl)-8-[5-(2-pyridinyl)-1H-benzimidazol-2-yl]-1-oxa-3-azaspiro[4.5]decan-2-one;
(trans)-3-(2-fluorophenyl)-8-[5-(1,3-thiazol-4-yl)-1H-benzimidazol-2-yl]-1-oxa-3-azaspiro[4.5]decan-2-one;
(trans)-3-(2-fluorophenyl)-8-[5-(1,3-thiazol-2-yl)-1H-benzimidazol-2-yl]-1-oxa-3-azaspiro[4.5]decan-2-one;
(trans)-3-(2-fluorophenyl)-8-[5-(2-pyrimidinyl)-1H-benzimidazol-2-yl]-1-oxa-3-azaspiro[4.5]decan-2-one;
8-(2-chloro-1H-purin-8-yl)-3-phenyl-1-oxa-3-azaspiro[4.5]decan-2-one;
(trans)-3-(6-fluoro-2-pyridinyl)-8-{5-[(trifluoromethyl)oxy]-1H-benzimidazol-2-yl}-1-oxa-3-azaspiro[4.5]decan-2-one;
(cis)-3-(6-fluoro-2-pyridinyl)-8-{5-[(trifluoromethyl)oxy]-1H-benzimidazol-2-yl}-1-oxa-3-azaspiro[4.5]decan-2-one;
3-(6-fluoro-2-pyridinyl)-8-[5-(trifluoromethyl)-1H-benzimidazol-2-yl]-1-oxa-3-azaspiro[4.5]decan-2-one;
3-(2-pyridinyl)-8-[6-(trifluoromethyl)-1H-imidazo[4,5-c]pyridin-2-yl]-1-oxa-3-azaspiro[4.5]decan-2-one;
3-(2-pyridinyl)-8-[6-(trifluoromethyl)-1H-imidazo[4,5-b]pyridin-2-yl]-1-oxa-3-azaspiro[4.5]decan-2-one.
   or pharmaceutically acceptable salts thereof.

Example compounds of the present invention include:
(trans)-3-phenyl-8-[5-(trifluoromethyl)-1H-benzimidazol-2-yl]-1-oxa-3-azaspiro[4.5]decan-2-one;
(trans)-3-phenyl-8-{5-[(trifluoromethyl)oxy]-1H-benzimidazol-2-yl}-1-oxa-3-azaspiro[4.5]decan-2-one;
(trans)-3-(2-fluorophenyl)-8-{5-[(trifluoromethyl)oxy]-1H-benzimidazol-2-yl}-1-oxa-3-azaspiro[4.5]decan-2-one;
(trans)-3-(4-fluorophenyl)-8-{5-[(trifluoromethyl)oxy]-1H-benzimidazol-2-yl}-1-oxa-3-azaspiro[4.5]decan-2-one;
2-[(trans)-2-oxo-3-phenyl-1-oxa-3-azaspiro[4.5]dec-8-yl]-1H-benzimidazole-5-carbonitrile;
(trans)-3-(6-methyl-2-pyridinyl)-8-{5-[(trifluoromethyl)oxy]-1H-benzimidazol-2-yl}-1-oxa-3-azaspiro[4.5]decan-2-one;
(trans)-8-[5-(3,5-dimethyl-4-isoxazolyl)-1H-benzimidazol-2-yl]-3-(2-pyridinyl)-1-oxa-3-azaspiro[4.5]decan-2-one;
   or pharmaceutically acceptable salts thereof.

In general, the compounds of structure (I) may be made according to the organic synthesis techniques known to those skilled in this field, as well as by the representative methods set forth in the Examples.

Compounds of formula (I), and salts and solvates thereof, may be prepared by the general methods outlined hereinafter. In the following description, the groups R₁, R₂, R₃, X, m, Z, G have the meanings as previously defined for compounds of formula (I) unless otherwise stated.

Compounds of formula (Ic) may be conveniently prepared, starting from compounds of formula (II), according to the following Scheme 1:

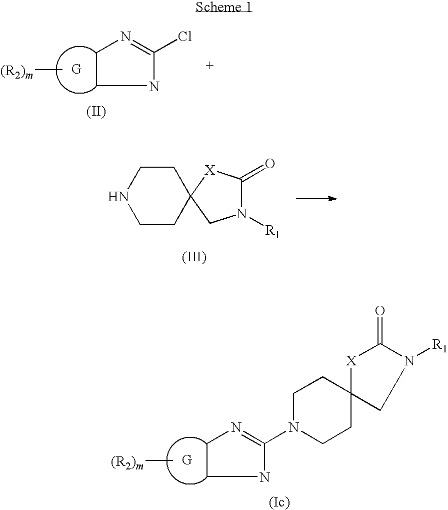

Compounds of formula (Ic), corresponding to the compounds of formula (I) where Z═N, can be prepared by reaction of a chloride of formula (II) and amines of formula (III), optionally in the presence of a base such as Hunig's base in a solvent such as butanol, DMF or dimethylsulfoxide at a temperature between 60° C. and 200° C. Chlorides of formula (II) are commercially available or can be prepared according to methods described in the literature e.g. J. Chem. Soc., 1963, 2930-7 and the references described therein. The preparation of amines of formula (III) where X═O is described in the Journal of Medicinal Chemistry, 1995, 38(19), 3772-9, U.S. Pat. No. 4,244,961 and WO9711940; amines of formula (III) where X═CH₂ are described in Journal of Medicinal Chemistry, 2004, 47(8), 2037-2061 and WO2005007656.

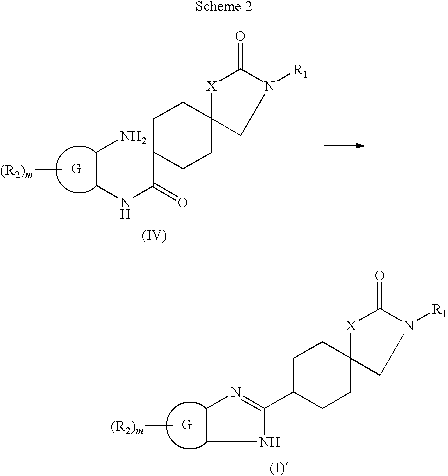

Compounds of formula (I)′, as defined above, can be prepared by reaction of an amino-amide of formula (IV), in the presence of an acid e.g. p-toluenesulfonic acid in a solvent mixture such as dioxane and toluene, preferably at a temperature between 60° C. and 200° C.

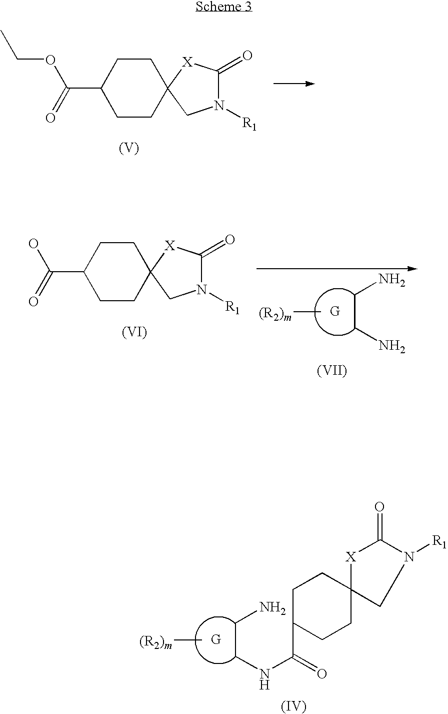

Compounds of formula (IV) can be prepared by reaction of a 1,2-aryl-diamine of formula (VII) and a carboxylic acid of formula (VI). Standard conditions for such transformations are described in Tetrahedron, 2005, 61(46), 10827-10852 and the references therein. Carboxylic acids of formula (VI) can be prepared from esters of formula (V) via hydrolysis with a reagent such as lithium hydroxide or sodium hydroxide in a suitable solvent (e.g. methanol/water or THF/water) followed by acidification with an acid (e.g. hydrochloric acid).

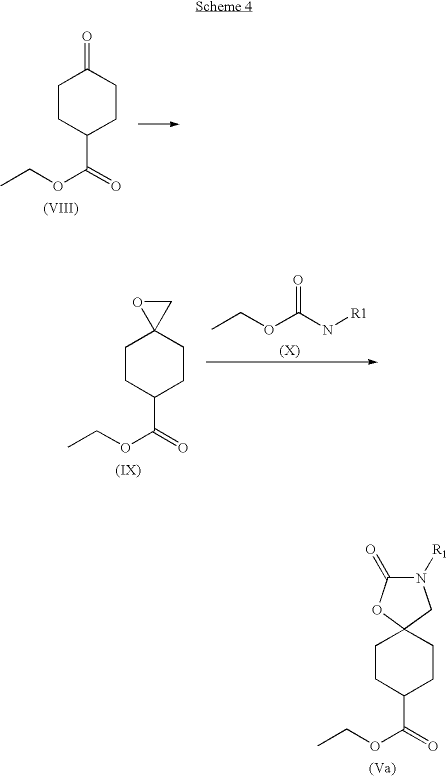

Esters of formula (Va), i.e. a compound of formula (V) wherein X is oxygen, can be prepared from an epoxide of formula (IX) and a carbamate of formula (X) in a solvent such as HPMA, DMPU or NMP in the presence of a base such as sodium tertiary-butoxide, sodium hydride or BEMP, preferably at a temperature greater than 100° C. An epoxide of formula (IX) can be prepared from a ketone (VIII), which is commercially available from e.g. Sigma-Aldrich Chemicals, by treatment with trimethylsulphoxonium iodide or trimethylsulphonium iodide in an aprotic solvent such as DMSO or acetonitrile in the presence of a base such as sodium hydride, potassium tertiary-butoxide or 2,8,9-triisobutyl-2,5,8,9-tetraaza-1-phosphabicyclo-[3.3.3]undecane. Carbamates of formula (X) are commercially available from e.g. Sigma-Aldrich Chemicals.

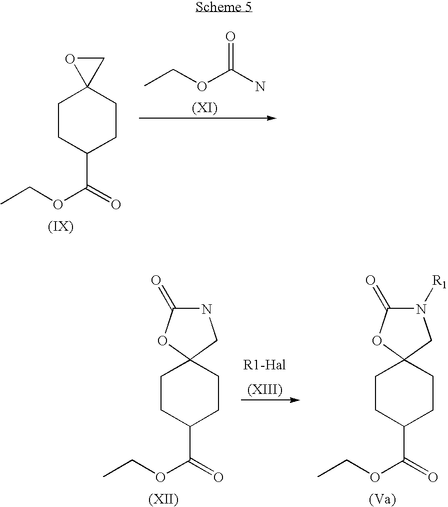

Esters of formula (Va) can be prepared from esters of formula (XII) and an alkyl halide or aryl halide of formula (XIII). Where R₁=aryl, suitable reactions conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Where R₁=alkyl, suitable conditions include the use of a base such as sodium hydride in a solvent such as THF or DMF. Aryl halides and alkyl halides of formula (XIII) are commercially available from e.g. Sigma-Aldrich Chemicals. Esters of formula (XII) can be prepared from an epoxide of formula (IX) and a carbamate of formula (XI) in a solvent such as HPMA, DMPU or NMP in the presence of a base such as sodium tertiary-butoxide, sodium hydride or BEMP, preferably at a temperature greater than 100° C. A carbamate of formula (XI) is commercially available from e.g. Sigma-Aldrich Chemicals.

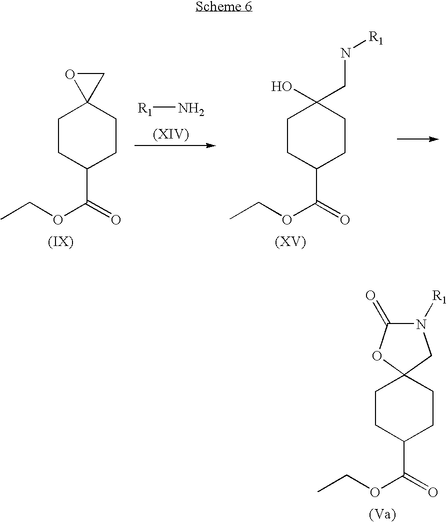

Alternatively, esters of formula (Va) can be prepared from amino-alcohols of formula (XV) and a reagent such as phosgene, triphosgene, carbonyl di-imidazole, disuccinimidyl carbonate, carbon dioxide, an alkylchloroformate e.g. benzyl chloroformate or ethyl chloroformate, an aryl chloroformate e.g. phenyl chloroformate or a dialkyl pyrocarbonate e.g. di-tertiary-butyl di-carbonate (Boc anhydride), optionally in the presence of a base such as triethylamine in a solvent such as dichloromethane. Amino-alcohols of formula (XV) can be prepared from an epoxide of formula (IX) and amines of formula (XIV) in a protic solvent such as tertiary-butanol or ethoxyethanol at temperatures greater than 100° C. Amines of formula (XIV), such as aniline, are commercially available from e.g. Sigma-Aldrich Chemicals.

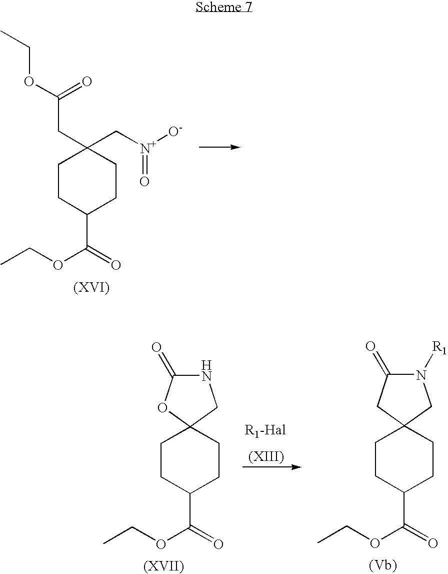

Esters of formula (Vb), i.e. a compound of formula (V) wherein X is carbon, can be prepared from a lactam of formula (XVII) and an alkyl halide or aryl halide of formula (XIII). Where R₁=aryl, suitable reactions conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Where R₁=alkyl, suitable conditions include the use of a base such as sodium hydride in a solvent such as THF or DMF. Aryl halides and alkyl halides of formula (XIII) are commercially available from e.g. Sigma-Aldrich Chemicals. A lactam of formula (XVII) can be prepared from a nitro-ester of formula (XVI) via reaction with a reducing agent e.g. iron powder/ammonium chloride in a solvent mixture such as ethanol-water at a temperature from 25° C. to 80° C.

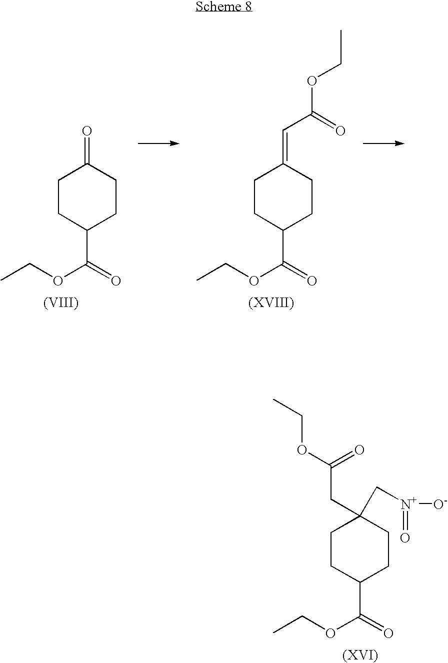

A nitro-ester of formula (XVI) can be prepared from an alkene of formula (XVIII) and nitromethane in the presence of a reagent such as TBAF in a solvent such as THF optionally with heating to 40° C. An alkene of formula (XVIII) can be prepared from a ketone of formula (VIII) via e.g. reaction with triethyl phosphonoacetate in the presence of sodium hydride in a solvent such as THF. A ketone of formula (VIII) is commercially available from e.g. Sigma-Aldrich Chemicals.

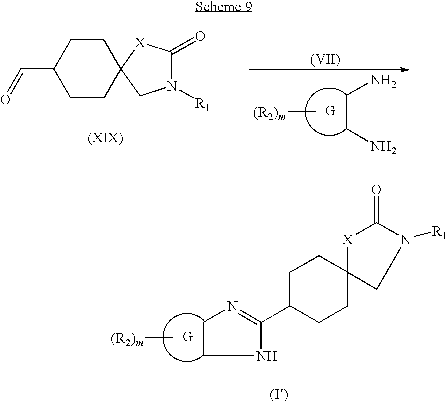

Alternatively, compounds of formula (I)′, can be prepared by reaction of a 1,2-aryl-diamine of formula (VII) and an aldehyde of formula (XIX) according to procedures described in Tetrahedron Letters, 2005, 46(25), 4315-4319.

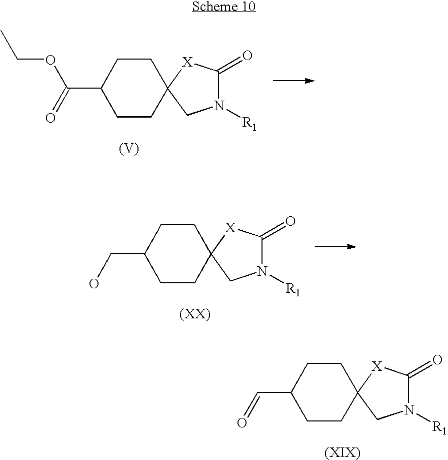

Aldehydes of formula (XIX) can be prepared by oxidation of alcohols of formula (XX) using a reagent such as Dess-Martin periodinane, DMPX, TPAP or ‘Swern’ oxidation conditions (oxalyl chloride/dimethyl sulfoxide in the presence of an amine base e.g. triethylamine or Hunig's base). Alcohols of formula (XX) can be prepared from esters of formula (V) via reduction with a reagent such as lithium aluminium hydride at a temperature below 0° C. in an aprotic solvent such as THF.

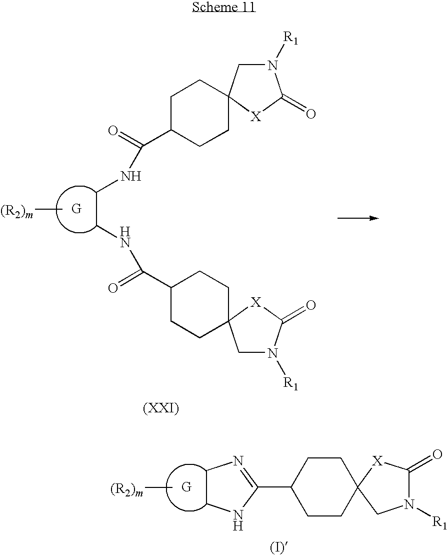

Alternatively, compounds of formula (I)′ can be prepared from di-amides of formula (XXI) by heating with a reagent such as polyphosphoric acid at a temperature of e.g. >100 C.

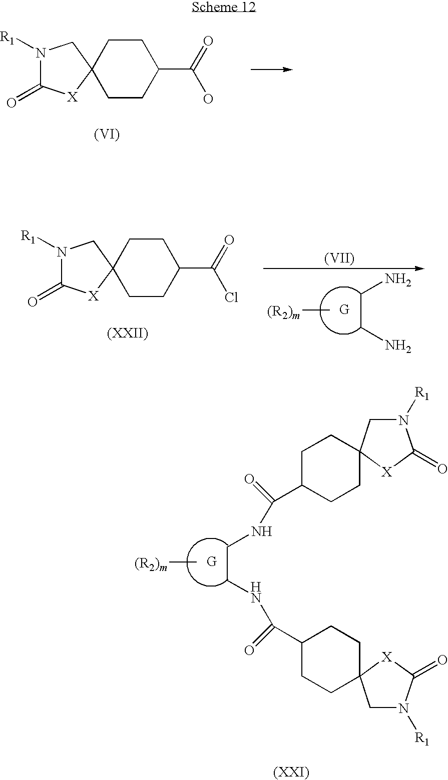

Di-amides of formula (XXI) can be prepared from acid chlorides of formula (XXII) and di-amines of formula (VII). Acid chlorides of formula (XXII) can be prepared from carboxylic acids of formula (VI) and a reagent such as oxalyl chloride, optionally in the presence of a catalyst such as dimethylformamide in a solvent such as DCM.

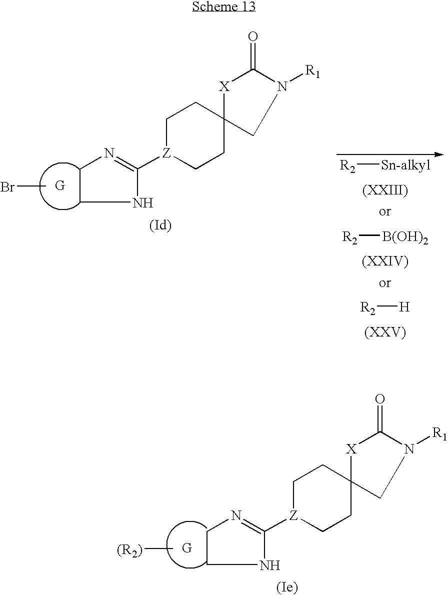

Compounds of formula (Ie), i.e. a compound of formula (I) wherein R₂ is aryl, heteroaryl or heterocycle which may be substituted by one or more: halogen, C₁-C₄ alkyl, C₁-C₄ alkoxy, C₁-C₄ haloalkyl, C₁-C₄ haloalkoxy, cyano can be prepared from aryl bromides of formula (Id), i.e. a compound of formula (I) wherein R₂ is bromine, by reaction with an organostannane of formula (XXIII) or a boronic acid of formula (XXIV). Suitable reaction conditions have been described in “The Stille Reaction”, Organic Reactions (New York) (1997), 50 1-652 and “Transition Metals for Organic Synthesis” (2nd Edition) (2004), 1, 211-229, and the references therein. Organostannanes of formula (XXIII) and boronic acids of formula (XXIV) are commercially available from e.g. Sigma-Aldrich Chemicals. Some compounds of formula (Ie) can be prepared from aryl bromides of formula (Id) by reaction with a heterocycle as above defined or heteroaromatic compound of formula (XXV). Suitable reaction conditions have been described in ‘Metal-Catalyzed Cross-Coupling Reactions (2nd Edition), 2004, 2, 699-760; Angewandte Chemie, International Edition, 2003, 42(44), 5400-5449 and the references therein. Compounds of formula (Id) can be prepared as described in Schemes 1-3.

In the preparation of some compounds of formula (Ie) it could be necessary to protect the free “imidazole” NH group with a suitable protecting group, e.g. t-butyloxycarbonyl (Boc), before the described C—C coupling reaction. Subsequent N-deprotection can be performed under acidic reaction conditions e.g. treatment with HCl or trifluoroacetic acid in a solvent such as DCM or MeOH.

Those skilled in the art will appreciate that in the preparation of the compounds of the invention it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl).

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁶I.

Compounds of the present invention that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Isotopically labelled compounds of formula (I) and following of this invention can generally be prepared by carrying out the procedures disclosed in the Schemes and/or in the Examples below, by substituting a readily available isotopically labelled reagent for a non-isotopically labelled reagent.

Compounds of the present invention are antagonists of the NPY Y5 receptor and as such are useful for the prevention and treatment of disorders or diseases associated with the NPY Y5 receptor sub-type, preferably for the treatment of eating disorders such as obesity, anorexia nervosa and bulimia nervosa, and other abnormal conditions, such as diabetes, hypertension, hyperlipemia, hypercholesterolemia, congestive heart failure, renal dysfunction, sexual/reproductive disorders, depression, anxiety, shock, epileptic seizure, memory loss, sleep disturbance, pain, migraine, cerebral hemorrhage, nasal congestion, gastrointestinal disorders, arthritis and immunodeficiency syndrome.

The compounds of the present invention may also be used in combination with other anti-obesity agents for increased efficacy in the prevention and treatment of eating disorders. Such agents would include, but not be limited to: sibutramine; dexfenfluramine; leptin; growth hormone secretagogue antagonists such as those disclosed and specifically described in U.S. Pat. No. 5,536,716; melanocortin agonists such as elanotan II; Beta-3 agonists such as those disclosed and specifically described in patent publications WO94/18161, WO95/29159, WO97/46556, WO98/04526 and WO98/32753; 5HT-2 agonists; orexin antagonists; melanin concentrating hormone antagonists; galanin antagonists; CCK agonists; GLP-1 agonists; corticotrophin releasing hormone agonists; Y1 antagonists, and CB1 antagonists.

More particularly, compounds of the present invention are useful as agents for the treatment and/or prophylaxis of eating disorders such as a binge eating disorder.

The method of treatment of this invention comprises a method of antagonizing the NPY Y5 receptor and treating NPY Y5 receptor mediated diseases by administering to a patient in need of such treatment a non-toxic therapeutically effective amount of a compound of this invention that selectively antagonizes the NPY Y5 receptor in preference to the other NPY receptors.

Within the context of the present invention, the terms describing some indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type:

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9);

In a further embodiment the invention provides the use of a compound of formula (I), or a pharmaceutically acceptable salt or solvate thereof, in the preparation of a medicament for the treatment of a binge eating disorder.

In a further embodiment the present invention provides a method of treatment of a mammal suffering from a binge eating disorder, which comprises administering to said subject an effective amount of a compound of formula (I) or a pharmaceutically acceptable salt or solvate thereof.

In a further embodiment the invention provides the use of a compound of formula (I), or a pharmaceutically acceptable salt or solvate thereof, in the preparation of a medicament for the treatment of obesity.

In a further embodiment the present invention provides a method of treatment of a mammal suffering from obesity, which comprises administering to said subject an effective amount of a compound of formula (I) or a pharmaceutically acceptable salt or solvate thereof.

Compounds of formula (I) can be administered orally or parenterally and may be formulated in the form suitable for administration to provide an agent for treatment of various diseases related to NPY, which include, for example, cardiovascular disorders (for example hypertension, nephropathy, heart disease, vasospasm, arteriosclerosis), central nervous system disorders (for example bulimia, depression, anxiety, seizure, epilepsy, dementia, pain, alcoholism, drug withdrawal), metabolic diseases (for example obesity, diabetes, hormone abnormality, hypercholesterolemia, hyperlipidemia), sexual and reproductive dysfunction, gastro-intestinal motility disorder, respiratory disorder, inflammation or glaucoma and the like, preferably, bulimia, obesity, diabetes and the like.

While it is possible that, for use in therapy a therapeutically effective amount of a compound of formula (I), or a pharmaceutically acceptable salt or solvate thereof, may be administered as the raw chemical, it is preferable to present the active ingredient as a pharmaceutical composition. Thus, in a further embodiment the invention provides a pharmaceutical composition comprising a compound of formula (I), or a pharmaceutically acceptable salt or solvate thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of formula (I), or a pharmaceutically acceptable salt or solvate thereof, with one or more pharmaceutically acceptable carriers, diluents or excipients.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

The compounds of the present invention can be used in combination with other agents useful for treating metabolic and/or eating disorders. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating metabolic and/or eating disorders includes in principle any combination with any pharmaceutical composition useful for treating metabolic and/or eating disorders.

A therapeutically effective amount of a compound of formula (I), or a pharmaceutically acceptable salt or solvate thereof will depend upon a number of factors including, for example, the age and weight of the human or other mammals, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the NPY Y5 receptor will generally be in the range of 0.1 to 100 mg/kg body weight of recipient (mammal) per day and more usually in the range of 1 to 10 mg/kg body weight per day. Thus, for a 70 kg human adult, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt or solvate thereof may be determined as a proportion of the effective amount of the compound of formula (I) per se.

A compound of formula (I), or a pharmaceutically acceptable salt or solvate thereof for use in the instant invention may be used in combination with one or more other therapeutic agents. The invention thus provides in a further embodiment a combination comprising a compound of formula (I), or a pharmaceutically acceptable salt or solvate thereof together with a further therapeutic agent, which may be for example an additional anti-obesity agent. In a yet further embodiment the invention also provides the use of a combination comprising a compound of formula (I), or a pharmaceutically acceptable salt or solvate thereof with a further therapeutic agent in the treatment of disorders mediated by the NPY Y5 receptor.

When a compound of formula (I), or a pharmaceutically acceptable salt or solvate thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

The following Examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. This invention is meant to include such equivalents.

### EXPERIMENTAL

The invention is illustrated by the Compounds described below.

### Abbreviations

DMAP 4-dimethylaminopyridine

### DIPEA N,N-diisopropylethylamine

TEA triethylamine
TFA trifluoroacetic acid
EtOAc ethyl acetate
EDC.HCl N-(3-dimethylaminopropyl)-N′-ethylcarbodiimide hydrochloride
EDAC N-(3-dimethylaminopropyl)-N′-ethylcarbodiimide hydrochloride
EDC N-(3-dimethylaminopropyl)-N′-ethylcarbodiimide
HOBt.H₂O-hydroxybenzyltriazole hydrate
DMSO dimethylsulfoxide
DCM dichloromethane

### DMF N,N-dimethylformamide

HATU (O-7-azabenzotriazol-1-yl)-N,N,N′,N′-tetramethyluronium hexafluorophosphate)
THF tetrahydrofuran
MDAP mass-directed autopurification
MeOH methanol
Et₂O diethyl ether
r.t. room temperature
BuOH butanol
DMPU 1,2-dimethyl-3,4,5,6-tetrahydro-2(1H)-pyrimidone

Compounds were named using ACD/Name PRO 6.02 chemical naming software (Advanced Chemistry Development Inc., Toronto, Ontario, M5H2L3, Canada).

### Analytical Equipment

Proton Magnetic Resonance (NMR) spectra are recorded either on Varian instruments at 300, 400, 500 or 600 MHz, or on Bruker instruments at 300 or 400 MHz. Chemical shifts are reported in ppm (δ) using the residual solvent line as internal standard. Splitting patterns are designated as: s, singlet; d, doublet; t, triplet; q, quartet; m, multiplet; br, broad. The NMR spectra are recorded at a temperature ranging from 25 to 90° C. When more than one conformer is detected the chemical shifts for the most abundant one are reported.

Mass spectra (MS) are taken on a 4 II triple quadrupole Mass Spectrometer (Micromass UK) or on a Agilent MSD 1100 Mass Spectrometer, operating in ES(+) and ES(−) ionization mode. The usage of this methodology is indicated by “MS”.

HPLC-Mass spectra (HPLC-MS) are taken on a Agilent LC/MSD 1100 Mass Spectrometer, operating in ES(+) and ES(−) ionization mode coupled with HPLC instrument Agilent 1100 Series [LC/MS-ES (+): analysis performed on a Supelcosil ABZ+Plus (33×4.6 mm, 3 m) (mobile phase: 100% [water+0.1% formic acid] for 1 min, then from 100% [water+0.1% formic acid] to 5% [water+0.1% formic acid] and 95% [acetonitrile] in 5 min, finally under these conditions for 2 min; T=40° C.; flow=1 mL/min; LC/MS-ES (−): analysis performed on a Supelcosil ABZ+Plus (33×4.6 mm, 3 m) (mobile phase: 100% [water+0.05% ammonia] for 1 min, then from 100% [water+0.05% ammonia] to 5% [water+0.05% ammonia] and 95% [acetonitrile] in 5 min, finally under these conditions for 2 min; T=40° C.; flow=1 mL/min]. In the mass spectra only one peak in the molecular ion cluster is reported. The usage of this methodology is indicated by “HPLC-MS 1” in the analytical characterization of the described compounds.

Alternatively, HPLC-MS measurements are carried out using a Platform LCZ™ single quadrupole Mass Spectrometer (Micromass-Waters), coupled with an HPLC system Agilent 1100 Series. The experimental conditions are: column XBridge C18, (5 μm 4.6×50 mm), column temperature 30° C., mobile phase, A=water+0.1% TFA and B=MeCN, gradient, t=0 min 0% (B) to 60% (B) in 1.5 min to 95% (B) in 3.5 min lasting for 1.5 min (t=6.60 min 0% B stop time=7.0 min), flow rate 2 ml/min, DAD UV range 210 to 350 nm, MS ionisation mode, positive electrospray (ES+), MS range 110 to 1100 atomic mass unit. The usage of this methodology is indicated by “HPLC-MS 2” in the analytical characterization of the described compounds.

Alternatively, HPLC-MS measurements are carried out according to the following protocol.

### Column

The column used is a Waters Atlantis, the dimensions of which are 4.6 mm×20 mm. The stationary phase particle size is 3 μm. The column temperature is ambient.

### Solvents

A: Aqueous solvent=Water+0.1% Formic Acid
B: Organic solvent=Acetonitrile+0.1% Formic Acid
Needle wash solvent=Methanol

### Methods

A generic method is used with an injection volume of 2 μl and a 5.5 minute runtime using the following gradient:—

| Time | Solvent B % | Flowrate ml/min |
|---|---|---|
| 0.00 | 3 | 1.0 |
| 0.70 | 3 | 1.0 |
| 4.50 | 100 | 1.0 |
| 5.30 | 100 | 1.0 |
| 5.40 | 3 | 1.0 |
| 5.50 | 3 | 1.0 |

### Flowrate

The gradient flowrate is 1.0 mlmin⁻¹.

The use of this procedure is indicated by ‘LCMS’ in the experimental text.

Alternatively, HPLC-MS measurements were carried out according to the following protocol.

### Column

The column used is a Waters Xterra, the dimensions of which are 2.1 mm×30 mm. The stationary phase particle size is 3.5 μm. The column temperature is 25 degrees C.

### Solvents

A: Aqueous solvent=Water+0.1% Formic Acid
B: Organic solvent=95% Acetonitrile+0.1% Formic Acid
Needle wash solvent=40% Methanol, 40% IPA and 20% Water

### Methods

A generic method is used with an injection volume of 1 μl and a 4.5 minute runtime using the following gradient:—

| Time | Solvent B % | Flowrate ml/min |
|---|---|---|
| 0.00 | 0 | 1.0 |
| 0.1 | 0 | 1.0 |
| 3.10 | 100 | 1.0 |
| 3.80 | 100 | 1.0 |
| 3.90 | 0 | 1.0 |
| 4.00 | 0 | 2.0 |
| 4.40 | 0 | 2.0 |
| 4.50 | 0 | 1.0 |

### Flowrate

The gradient flow-rate is 1.0 mlmin

The used of this protocol is indicated by ‘LCMS (4.5 min)’ in the experimental text.

Total ion current (TIC) and DAD UV chromatographic traces together with MS and UV spectra associated with the peaks are taken also on a UPLC/MS Acquity™ system equipped with 2996 PDA detector and coupled to a Waters Micromass ZQ™ mass spectrometer operating in positive or negative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% formic acid/B−acetonitrile+0.075% formic acid, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “UPLC-MS” in the analytic characterization of the described compounds.

For reactions involving microwave irradiation, a Personal Chemistry Emrys™ Optimizer is used.

Flash silica gel chromatography is carried out on silica gel 230-400 mesh (supplied by Merck AG Darmstadt, Germany) or over Varian Mega Be-Si pre-packed cartridges or over pre-packed Biotage silica cartridges.

SPE-SCX cartridges are ion exchange solid phase extraction columns by supplied by Varian. The eluent used with SPE-SCX cartridges is usually methanol followed by 2N ammonia solution in methanol.

In a number of preparations, purification is performed using either Biotage manual flash chromatography (Flash+) or automatic flash chromatography (Horizon, SP1, Flashmaster) systems. All these instruments work with standard Biotage or Varian Silica cartridges.

SPE-Si cartridges are silica solid phase extraction columns supplied by Varian.

In a number of preparations, purification is performed on a Mass-Directed Autopurification (MDAP) system Fractionlynx™ equipped with Waters 2996 PDA detector and coupled with ZQ™ mass spectrometer (Waters) operating in positive and negative electrospray ionisation mode ES+, ES− (mass range 100-1000)

A set of acidic as well as basic semi-preparative gradients have been used:

METHOD A: Chromatographic Acidic conditions for up to 30 mg of crude:
Column: 100×21.2 mm Supelcosil™ ABZ+Plus (5 μm particle size)
Mobile phase: A[water+0.1% formic acid]/B[acetonitrile+0.1% formic acid]
Flow rate: 20 ml/min
Gradient: 5% B for 1 min, 95% B in 9 min, 100% B in 3.5 min
METHOD B: Chromatographic Acidic conditions for up to 100 mg of crude:
Column: 150×30 mm XTerra Prep MS C18 (10 μm particle size)
Mobile phase: A[water+0.1% formic acid]/B [acetonitrile+0.1% formic acid]
Flow rate: 40 mL/min
Gradient: 1% B to 100% B in 7 min lasting for 7.5 min.
METHOD C: Chromatographic Basic conditions for up to 100 mg of crude
Column: 150×30 mm XTerra Prep MS C18 (10 μm particle size)
Mobile phase: A-water+10 mM ammonium carbonate (adjusted to pH 10 with ammonia)/B−acetonitrile
Flow rate: 40 mL/min
Gradient: 10% B for 0.5 min, 95% B in 12.5 min

Alternatively, mass directed HPLC for compound purification is performed using the following protocol.

### Column

The columns used are Waters Atlantis, the dimensions of which are 19 mm×100 mm with a stationary phase particle size of 5 μm.

### Solvents

A: Aqueous solvent=Water+0.1% Trifluoroacetic Acid
B: Organic solvent=Acetonitrile+0.1% Trifluoroacetic Acid
Make up solvent=Methanol: Water (0.1% Formic Acid) 80:20
Needle rinse solvent=Methanol

### Methods

There are five methods used depending on the analytical retention time of the compound of interest. They have a 20 minute runtime, which comprises of a 15 minute gradient followed by a 5 minute column flush and re-equilibration step.

| LCMS retention time | Gradient used |
|---|---|
| 1.8 to 2.1 minutes | Linear gradient, 0-30% B |
| 2.1 to 2.6 minutes | Linear gradient, 10-45% B |
| 2.6 to 3.1 minutes | Linear gradient, 15-65% B |
| 3.1 to 4.1 minutes | Linear gradient, 30-75% B |
| Greater than 4.1 minutes | Linear gradient, 50-100% B |

### Flow Rate

All of the above methods have a flow rate of 20 ml.min⁻¹

The use of this protocol is indicated by ‘mass-directed HPLC’ in the experimental text.

All reactions are monitored by thin-layer chromatography on 0.25 mm E. Merck silica gel plates (60E-254), visualised with UV light, iodine, 5% ethanolic phosphomolybdic acid, ninhydrin solution or vanillin solution.

### Intermediates

### Intermediate 1: 2-Chloro-5-(trifluoromethyl)-1H-benzimidazole

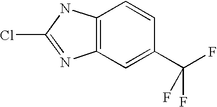

5-(Trifluoromethyl)-1,3-dihydro-2H-benzimidazol-2-one (Intermediate 2, 3.76 g, 18.6 mmol) and phosphoric trichloride (56 ml) were mixed at 95° C. for 18 hours. The solvent was stripped off and the residue treated with toluene (3×30 ml) and evaporated to dryness at 50° C. The resulting solid was recrystallised from EtOAc. The precipitate was filtered off, washed with Et₂O and dried to give the title compound (3.75 g, 17.0 mmol, 91%). 1H-NMR (400 MHz, DMSO-d6): δ 7.90 (1H, s), 7.72 (1H, d), 7.56 (1H, dd); UPLC-MS: 0.67 min, m/z 221 [M+H]+.

### Intermediate 2: 5-(trifluoromethyl)-1,3-dihydro-2H-benzimidazol-2-one

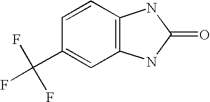

Commercially available 4-(trifluoromethyl)-1,2-benzenediamine (5.832 g, 33.11 mmol) was dissolved in THF at r.t. and carbonyldiimidazole (5.905 g, 36.42 mmol, 1.1 eq) was added. After stirring at r.t. for 18 hours, the mixture was diluted with EtOAc (200 ml) and washed with aqueous NH₄Cl (50 ml), aqueous NaHCO₃ (50 ml) and brine (50 ml). The organic phase was dried (Na₂SO₄), filtered and evaporated. The residue was purified by silica gel chromatography eluting with cyclohexane/EtOAc:1/1 to pure EtOAc to give the title compound (4.10 g, 20.28 mmol, 61%). 1H-NMR (400 MHz, DMSO-d6): δ 11.08 (2H, br s), 7.29 (1H, d), 7.16 (1H, s), 7.09 (1H, d); UPLC-MS: 0.57 min, m/z 203 [M+H]+.

### Intermediate 3: (Cis)-N,N′-[4-(trifluoromethyl)benzene-1,2-diyl]bis(2-oxo-3-phenyl-1-oxa-3-azaspiro[4.5]decane-8-carboxamide)

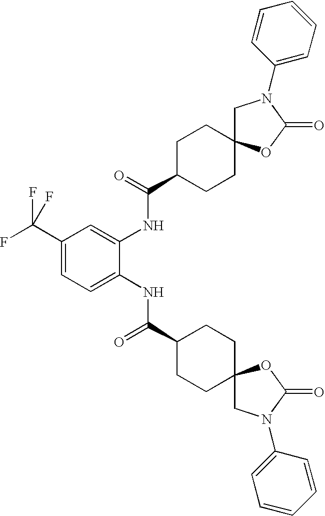

To a stirred solution of (cis)-2-oxo-3-phenyl-1-oxa-3-azaspiro[4.5]decane-8-carboxylic acid (Intermediate 4, 0.22 mmol) and oxalyl chloride (23 μl) in DCM (1 ml) was added dimethylformamide (1 μl). The mixture was stirred 1 hour, then a solution of 4-trifluoromethylphenyl-1,2-diamine (77 mg) in DCM (1 ml) was added dropwise. The mixture was stirred 1 hour, then left to stand 70 hours. The mixture was partitioned between EtOAc and water. The organic layer was washed (water, brine) and concentrated under vacuum. The residue was purified by column chromatography (silica gel; cyclohexane-ethyl acetate, 9:1 to 1:4) to give the title compound (28.7 mg). 1H-NMR (400 MHz, CDCl₃): δ 8.83 (2H, m), 7.66 (1H, br s), 7.58-7.48 (5H, m), 7.44-7.31 (5H, m), 7.19-7.11 (2H, m), 3.76 (4H, br s), 2.45 (2H, m), 2.24-1.92 (12H, m) and 1.76-1.58 (4H, m); UPLC-MS: 0.82 min, m/z 691 [M+H]+.

### Intermediate 4: (Cis)-2-oxo-3-phenyl-1-oxa-3-azaspiro[4.5]decane-8-carboxylic acid

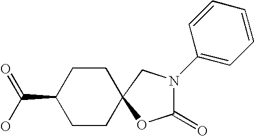

A mixture of 1,1-dimethylethyl (cis)-2-oxo-3-phenyl-1-oxa-3-azaspiro[4.5]decane-8-carboxylate (Intermediate 5, 74 mg, 0.22 mmol), DCM (2 ml) and TFA (0.2 ml) was shaken for 2 hours then concentrated under a stream of nitrogen while heating at 40° C. The residue was dried under vacuum at 40° C. to give the title compound. 1H-NMR (400 MHz, CDCl₃): δ 10.82 (1H, br s), 7.52 (2H, d J 8 Hz), 7.38 (2H, t, J 8 Hz), 7.16 (1H, J 8 Hz), 3.76 (2H, s), 2.50-2.37 (1H, m), 2.21-2.13 (2H, m), 2.08-1.94 (4H, m) and 1.71-1.59 (2H, m).

Alternatively, Intermediate 4 was prepared according to the following procedure.

### Intermediate 4: (Cis)-2-Oxo-3-phenyl-1-oxa-3-azaspiro[4.5]decane-8-carboxylic acid

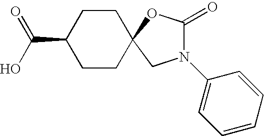

To a stirred solution of ethyl (cis)-2-oxo-3-phenyl-1-oxa-3-azaspiro[4.5]decane-8-carboxylate (0.45 g, 1.5 mmol) in methanol (10 ml) was added dropwise a solution of lithium hydroxide (0.18 g) in water (2 ml). The mixture was stirred 1 hour then left to stand for 18 hours. The mixture was acidified with dilute hydrochloric acid (1M) and extracted twice with ethyl acetate. The combined organic extracts were washed with water, filtered through a hydrophobic membrane and concentrated under vacuum to give (cis)-2-oxo-3-phenyl-1-oxa-3-azaspiro[4.5]decane-8-carboxylic acid (0.393 g, 96%) as a white solid. 1H-NMR (400 MHz, CDCl₃): δ 7.54 (2H, d, J 7.5 Hz), 7.38 (2H, t, J 7.5 Hz), 7.14 (1H, t, J 7.5 Hz), 3.75 (2H, s), 2.43 (1H, m), 2.19 (1H, m), 2.16 (1H, m), 2.08 (1H, m), 2.06-1.98 (3H, m) and 1.65 (2H, m); UPLC-MS: 0.62 min, m/z 274 [M−H]−.

### Intermediate 5: 1,1-Dimethylethyl (cis)-2-oxo-3-phenyl-1-oxa-3-azaspiro[4.5]decane-8-carboxylate

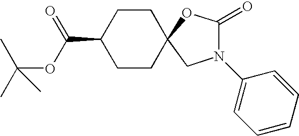

To a stirred mixture of (cis)-2-oxo-3-phenyl-1-oxa-3-azaspiro[4.5]decane-8-carboxylic acid (Intermediate 4, 0.38 g, 1.4 mmol), DMF (0.1 ml) and THF (8 ml) in a round-bottomed flask was added dropwise phosphorus oxychloride (0.15 ml, 1.6 mmol). The mixture was heated to 40° C. and stirred 2 hours. During this time, tetramethyl-ethylenediamine (0.73 ml, 4.8 mmol), tertiary butanol (0.20 ml, 2.1 mmol), lithium chloride (61 mg, 1.4 mmol) and THF (2 ml) were stirred together in a separate vial. The flask was cooled to room temperature and the contents of the vial were added dropwise to the stirred solution of the intermediate acid chloride. The mixture was heated to 35° C. and stirred for 18 hours. The mixture was diluted with water and extracted twice with EtOAc. The combined organic extracts were washed (water, dilute hydrochloric acid, water), filtered through a hydrophobic membrane and concentrated under vacuum to give the crude product (0.47 g). The crude product was purified by flash column chromatography (silica gel; cyclohexane-ethyl acetate, 10:1); the fractions containing only the slower-running isomer were combined and concentrated under vacuum to give 1,1-dimethylethyl (cis)-2-oxo-3-phenyl-1-oxa-3-azaspiro[4.5]decane-8-carboxylate as a viscous oil which crystallised on standing (74 mg). 1H-NMR (400 MHz, CDCl₃): δ 7.53 (2H, d, J 8 Hz), 7.36 (2H, t, J 8 Hz), 7.12 (1H, t, J 8 Hz), 3.71 (2H, s), 2.30-2.21 (1H, m), 2.15-2.07 (2H, m), 2.03-1.86 (4H, m), 1.60 (2H, td, J 13, 5 Hz) and 1.45 (9H, s); UPLC-MS: 0.85 min, m/z 276 [M-tBu+H]+, 331 [M+H]+ and 663 [2M+H]+.

### Intermediate 6: Ethyl (cis)-2-oxo-3-phenyl-1-oxa-3-azaspiro[4.5]decane-8-carboxylate

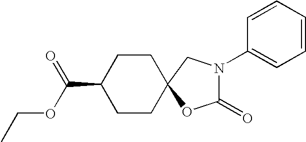

Ethyl 4-hydroxy-4-[(phenylamino)methyl]cyclohexanecarboxylate (prepared in an analogous manner to Intermediate 7, 190.5 mg, 0.68 mmole) was dissolved in anhydrous DCM (10 ml) and was cooled to −50° C. under nitrogen. At this temperature TEA (189.38 μl, 1.36 mmol) and triphosgene (100.7 mg, 0.34 mmol) were added. The reaction was stirred at −78° C. for 2.5 hours. More triphosgene (100.0 mg, 0.337 mmol) was added, and the mixture was stirred for a further 2 hours (until complete). The reaction was treated with a saturated aqueous solution of NH₄Cl and was extracted with DCM; the organic phase was dried over Na₂SO₄, filtered and concentrated under vacuum to give a residue (175 mg), which was purified by flash silica gel chromatography (compound Rf 0.27, cyclohexane:EtOAc 7:3). The fractions containing the lower-running product were combined and concentrated under vacuum to give the title compound (113.2 mg). 1H-NMR (500 MHz, CDCl₃): δ 1.28 (3H, t), 1.58-1.69 (2H, td), 1.91-2.09 (4H, m), 2.15 (2H, d), 2.30-2.42 (1H, m), 3.74 (2H, s), 4.15 (2H, q), 7.14 (1H, t), 7.38 (2H, t), 7.54 (2H, d); MS: m/z 304 [M+H]+.

### Intermediate 7: Ethyl 4-hydroxy-4-[(phenylamino)methyl]cyclohexanecarboxylate

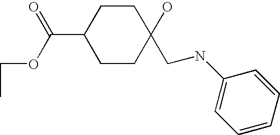

Ethyl 1-oxaspiro[2.5]octane-6-carboxylate (Intermediate 8, 704.5 mg, 3.82 mmol) was dissolved in t-BuOH (4 ml) and aniline (697 μl, 7.65 mmol, commercially available from e.g. Aldrich) was added. The reaction was stirred and heated at 150° C. under microwave irradiation for two 30 minute cycles. The mixture was poured into a saturated aqueous solution of NH₄Cl and extracted with EtOAc; the organic phase was dried on Na₂SO₄, filtered and evaporated in vacuo to give crude ethyl 4-hydroxy-4-[(phenylamino)methyl]cyclohexanecarboxylate (1.19 g), which was used without further purification. Another batch of the same compound prepared using an analogous method gave, 1H-NMR (400 MHz, CDCl₃): δ 1.22-1.30 (m, 3H), 1.36-2.02 (m, 9H), 2.23-2.34 (m, 1H), 2.45-2.54 (m, 1H), 3.09-3.13 (m, 1H), 3.16-3.21 (m, 1H), 4.10-4.20 (m, 2H), 6.65-6.77 (m, 3H), 7.14-7.24 (m, 2H).

### Intermediate 8: Ethyl 1-oxaspiro[2,5]octane-6-carboxylate

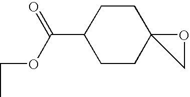

To a mixture of trimethylsulfoxonium iodide and potassium tert-butoxide (as reported in Synthetic Communications, 33(12), 2135-2143; 3.9 g, 11.76 mmol) was added a solution of ethyl 4-oxocyclohexanecarboxylate (1 g, 5.87 mmole, Aldrich) in DMSO (20 ml). The mixture was left to stir overnight at room temperature. The mixture was poured into water and extracted with diethyl ether; the organic phase was dried on Na₂SO₄, filtered and evaporated in vacuo to afford ethyl 1-oxaspiro[2.5]octane-6-carboxylate (704.5 mg, 65%), which was used without purification. Another batch of the same compound prepared using an analogous method gave 1H-NMR (400 MHz, CDCl₃): δ 1.20 (t, 3H), 1.27-1.49 (m, 2H), 1.63-2.04 (m, 6H), 2.26-2.28 (m, 1H), 2.49-2.59 (m, 2H), 4.06 (q, 2H).

Alternatively, Intermediate 8 was prepared using the following procedure:

A mixture of 2,8,9-triisobutyl-2,5,8,9-tetraaza-1-phosphabicyclo[3.3.3]undecane (1.14 ml, 3.94 mmol) and acetonitrile (15 ml) was added to a stirred suspension of trimethylsulphonium iodide (0.81 g, 3.97 mmol) and ethyl 4-oxocyclohexanecarboxylate (0.563 g, 3.31 mmol) at 0° C. The mixture was stirred at 0° C. for 30 minutes then allowed to warm to r.t. and stirred for a further 1 hour. The reaction mixture was concentrated under reduced pressure then diluted with diethyl ether. The resulting suspension was stirred for 30 minutes then filtered and the filter cake was washed with more diethyl ether. The combined ethereal phases were concentrated under reduced pressure and the residue was chromatographed on SiO₂ (Biotage 25+M column) eluting with a gradient of 5%-15% EtOAc/cyclohexane to give a ˜60:40, trans:cis mixture of the title compound as a colourless oil (250 mg). 1H-NMR (400 MHz, CDCl₃): δ 1.27 (3H both isomers, t), 1.37-1.52 (2H both isomers, m), 1.68-2.14 (6H both isomers, m), 2.35-2.48 (1H both isomers, m), 2.62 (2H cis isomer, s), 2.65 (2H trans isomer, s), 4.16 (2H both isomers, q).

Alternatively, Intermediate 8 was prepared using the following procedure:

Ethyl 4-oxocyclohexanecarboxylate (10.81 ml, 67.6 mmol) and trimethylsulfonium iodide (16.55 g, 81 mmol) were dissolved in dry acetonitrile (750 ml) under nitrogen. 2,8,9-Tris(2-methylpropyl)-2,5,8,9-tetraaza-1-phosphabicyclo[3.3.3]undecane (30.1 ml, 85 mmol) was added at r.t. The reaction mixture was stirred at r.t. for 1 hour. The solvent was removed under reduced pressure. The obtained solid was triturated with Et₂O (400 ml) and the suspension was filtered. The filter cake was washed with Et₂O (3×200 ml). The ether was evaporated and the residue was purified by silica gel chromatography eluting with cyclohexane/EtOAc:9/1 to 7/3 to afford the title compound (10.77 g, 58.5 mmol, 87%) as a mixture of the cis and trans-isomers (˜1:1, based on 1H-NMR). 1H-NMR (400 MHz, CDCl₃): δ 4.16 (2H, q), 2.65 (2H, d), 2.33-2.50 (1H, m), 2.05-2.16 (1H, m), 1.96-2.05 (1H, m), 1.83-1.92 (2H, m), 1.72-1.80 (2H, m), 1.49-1.58 (1H, m), 1.36-1.45 (1H, m), 1.24-1.32 (3H, t).

### Intermediate 9: (Trans)-N-[2-amino-5-(trifluoromethyl)phenyl]-2-oxo-3-phenyl-1-oxa-3-azaspiro[4.5]decane-8-carboxamide

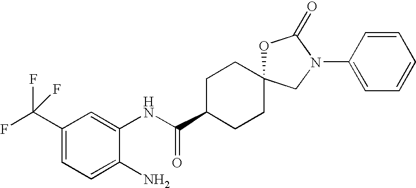

To a stirred solution of (trans)-2-oxo-3-phenyl-1-oxa-3-azaspiro[4.5]decane-8-carboxylic acid (Intermediate 10, 82 mg, 0.3 mmol) and 4-trifluoromethyl-1,2-diaminobenzene (commercially available, 79 mg) in pyridine (2 ml) was added EDAC (91 mg). The mixture was stirred 1 hour, then left to stand 18 hours. The mixture was concentrated under vacuum then partitioned between aqueous NaHCO₃ solution and ethyl acetate (×2). The combined organic extracts were washed with water, filtered through a hydrophobic membrane (Phase Sep) and concentrated under vacuum to give the crude product (196 mg). The crude was purified by chromatography on silica gel eluting with cyclohexane-ethyl acetate (1:0 to 0:1 gradient) to give the title compound (128 mg, 99%) as a 4:1 mixture with (trans)-N-[2-amino-4-(trifluoromethyl)phenyl]-2-oxo-3-phenyl-1-oxa-3-azaspiro[4.5]decane-8-carboxamide. 1H-NMR (400 MHz, CDCl₃): δ 8.31 (0.8H, br s), 8.17 (0.2H, br s), 7.54-6.75 (8H, m), 3.82 (2H, m), 2.50 (1H, m), 2.13-1.98 (4H, m), 1.90-1.71 (4H, m); UPLC-MS: 0.73 min, m/z 434 [M+H]+.

### Intermediate 10: (Trans)-2-oxo-3-phenyl-1-oxa-3-azaspiro[4.5]decane-8-carboxylic acid

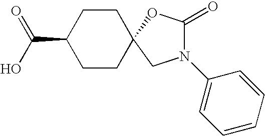

To a stirred solution of ethyl(trans)-2-oxo-3-phenyl-1-oxa-3-azaspiro[4.5]decane-8-carboxylate (Intermediate 11, 90 mg) in methanol (5 ml) was added dropwise aqueous sodium hydroxide solution (0.5M, 1.2 ml). The mixture was stirred 2 hours, left to stand overnight then concentrated under vacuum. The residue was partitioned between dilute HCl (10 ml) and DCM (20 ml). The organic layer was filtered through a hydrophobic membrane and concentrated under vacuum to give the title compound as a white foam (85 mg). 1H-NMR (400 MHz, CDCl₃): δ 7.55 (2H, d, J 8 Hz), 7.39 (2H, t, J 8 Hz), 7.15 (1H, t, J 8 Hz), 3.78 (2H, s), 2.62 (1H, septet, J 4 Hz), 2.21-2.12 (2H, m), 2.02-1.89 (4H, m), 1.88-1.77 (2H, m).

Alternatively, Intermediate 10 was prepared using the following procedure.

### Intermediate 10: (Trans)-2-oxo-3-phenyl-1-oxa-3-azaspiro[4.5]decane-8-carboxylic acid

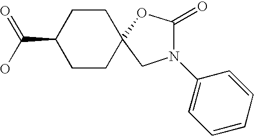

(Trans)-8-(hydroxymethyl)-3-phenyl-1-oxa-3-azaspiro[4.5]decan-2-one (prepared in an analogous manner to Intermediate 33, 0.396 g, 1.515 mmol) was dissolved in dry DCM (15 ml). Dess-Martin Periodinane (0.771 g, 1.818 mmol) was added in two portions (in about 15 minutes) and the resulting mixture was stirred at r.t. for 3.5 hours. The mixture was diluted with DCM (ca 20 ml) and 20 ml of 5% Na₂SO₃ solution in NaHCO₃ saturated aqueous solution was added and the mixture was stirred for 45 minutes. Then extraction was done with DCM (3×10 ml) to give extract 1, which was dried to give the aldehyde derivative (0.237 g, 60%). The aqueous was saturated with solid NaCl and extracted with DCM (3×50 ml) to give extract 2. Then the aqueous was acidified with 6N HCl and extracted with DCM (3×50 ml) to give extract 3. Extracts 2 and 3 were combined, dried (Na₂SO₄) and concentrated (rotary evaporator) to give 607 mg of a solid that was purified by silica chromatography (SP1, Biotage 25+M) eluting with cyclohexane:EtOAc 1:1 to 100% EtOAc to afford the title compound as a white solid (0.495 g of a crude 1:2 mixture of title compound:impurities by 1H-NMR); UPLC-MS: 0.60 min, m/z 276 [M+H]+.

### Intermediate 11: Ethyl(trans)-2-oxo-3-phenyl-1-oxa-3-azaspiro[4.5]decane-8-carboxylate

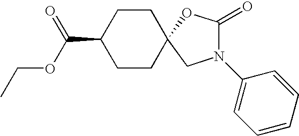

To a stirred solution of ethyl 4-hydroxy-4-[(phenylamino)methyl]cyclohexanecarboxylate (prepared in an analogous manner to Intermediate 7, 6:1 mixture of cis:trans isomers, 0.62 g) in DCM (15 ml) at −78° C. under nitrogen was added dropwise a solution of triphosgene (0.33 g) in DCM (15 ml). The mixture was stirred 1 hour then warmed slowly to r.t. The mixture was washed with aqueous NaHCO₃ solution, filtered through a hydrophobic membrane and concentrated under vacuum to give the crude product (0.827 g). The crude was purified by column chromatography on silica gel eluting with 4:1 cyclohexane-EtOAc; fractions containing the faster-running component were combined and concentrated under vacuum to give the title compound (97 mg). 1H-NMR (400 MHz, CDCl₃): δ 7.54 (2H, d, J 8 Hz), 7.36 (2H, t, J 8 Hz), 7.12 (1H, t, J 8 Hz), 4.15 (2H, q, J 7 Hz), 3.76 (2H, s), 2.50 (1H, septet, J 4 Hz), 2.14-2.05 (2H, m), 1.99-1.83 (4H, m), 1.80-1.69 (2H, m), 1.27 (3H, t, J 7 Hz).

Alternatively, Intermediate 11 was prepared according to the following procedure:

### Intermediate 11: Ethyl(trans)-2-oxo-3-phenyl-1-oxa-3-azaspiro[4.5]decane-8-carboxylate

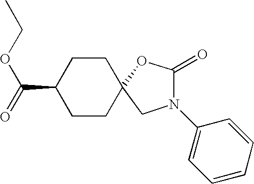

In a round-bottomed flask, ethyl 2-oxo-1-oxa-3-azaspiro[4.5]decane-8-carboxylate (Intermediate 40, 2.5 g, 11.00 mmol) was dissolved in toluene (25 ml), iodobenzene (2.462 ml, 22.00 mmol), cesium carbonate (8.96 g, 27.5 mmol), copper(I) iodide (0.105 g, 0.550 mmol) and trans-1,2-diaminocyclohexane (0.132 ml, 1.100 mmol) were added and the mixture was vigorously stirred at 80° C. overnight (˜16 hours). TLC analysis (cyclohexane:EtOAc 1:1) showed only traces of target material. The mixture was poured in a closed tube, copper(I) iodide (0.105 g, 0.550 mmol) and trans-1,2-diaminocyclohexane (0.132 ml, 1.100 mmol) were added and the mixture was further stirred at 80° C. for 7 hours, then was left to stand at room temperature for 4 days. TLC (Cyclohexane:EtOAc 6:4) showed presence of starting material. Copper(I) iodide (0.105 g, 0.550 mmol) was added and the mixture was stirred at 80° C. for 24 hours. The mixture was diluted with EtOAc (100 ml) and washed with water. The aqueous layer was extracted with EtOAc (3×50 ml). The organics were combined and dried to give a crude that was submitted to silica chromatography (Biotage SP1, 40+M, then a second one on a 25+M) eluent, cyclohexane:EtOAc 100:0 to 50:50 to afford the title compound (0.611 g, 17%), the corresponding cis isomer (0.852 g, 24%) and unreacted starting material (0.534 g, 21%). 1H-NMR (400 MHz, CDCl₃): δ 1.29 (t, 3H), 1.71-1.83 (m, 2H), 1.85-2.03 (m, 4H), 2.05-2.18 (m, 2H), 2.47-2.57 (m, 1H), 3.79 (s, 2H), 4.17 (q, 2H), 7.12-7.18 (m, 1H), 7.36-7.43 (m, 2H), 7.53-7.60 (m, 2H); UPLC-MS: 0.75 min, m/z 304 [M+H]+.

### Intermediate 12: 2,5,6-Trichloro-1H-benzimidazole

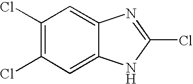

A mixture of 5,6-dichloro-1,3-dihydro-2H-benzimidazol-2-one (Intermediate 13, 0.75 g, 3.7 mmol) and phosphorus oxychloride (1.6 ml) was heated to 110° C. and stirred for 5 hour. The mixture was cooled to room temperature and quenched by addition of ice. The mixture was left to stand 30 minutes, then basified to pH 9 with aqueous ammonium hydroxide solution. The resulting precipitate was filtered off, washed with water and dried under vacuum. The residue was dissolved in ethyl acetate, and filtered. The filtrate was concentrated under vacuum to give the title compound (0.25 g). 1H-NMR (400 MHz, DMSO-d6): δ 7.82 (2H, s); UPLC-MS: 0.68 min, m/z 221 [M+H]+.

### Intermediate 13: 5,6-Dichloro-1,3-dihydro-2H-benzimidazol-2-one

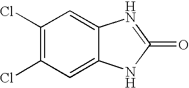

A mixture of 4,5-dichloro-1,2-diaminobenzene (commercially available, 1.0 g, 5.6 mmol), carbonyldiimidazole (1.0 g) and THF (4 ml) was heated to 150° C. in a microwave reactor and stirred for 10 minutes. The mixture was heated to 180° C. and stirred for a further 10 minutes. The mixture was cooled to room temperature and concentrated under vacuum. The residue was suspended in dilute hydrochloric acid and filtered. The filter-cake was washed with water and cyclohexane then dried under vacuum to give the title compound as an orange solid (0.98 g, 85%). 1H-NMR (400 MHz, DMSO-d6): δ 10.90 (2H, m), 7.10 (2H, m); UPLC-MS: 0.58 min, m/z 203 [M+H]+.

### Intermediate 14: 2-Chloro-5-fluoro-1H-benzimidazole

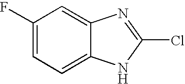

A mixture of 5-fluoro-1,3-dihydro-2H-benzimidazol-2-one (Intermediate 15, 0.75 g, 4.1 mmol) and phosphorus oxychloride (2.1 ml) was heated to 110° C. and stirred for 5 hours. The mixture was cooled to room temperature and quenched by addition of ice. The mixture was left to stand 30 minutes, then basified to pH 9 with aqueous ammonium hydroxide solution. The resulting precipitate was filtered off, washed with water and dried under vacuum. The residue was dissolved in ethyl acetate, and filtered. The filtrate was concentrated under vacuum to give the title compound (0.49 g). 1H-NMR (400 MHz, DMSO-d6): δ 7.52 (1H, dd, J 9, 5 Hz), 7.35 (1H, dd, J 9, 2.5 Hz) and 7.08 (1H, ddd, J 10, 9, 2.5 Hz). UPLC-MS: 0.55 min, m/z 171 [M+H]+.

(Alternatively, 5-fluoro-2-chloro-1H-benzimidazole is commercially available)

### Intermediate 15: 5-Fluoro-1,3-dihydro-2H-benzimidazol-2-one

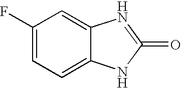

A mixture of 4-fluoro-1,2-diaminobenzene (commercially available, 1.0 g, 7.9 mmol), carbonyldiimidazole (1.4 g) and THF (4 ml) was heated to 150° C. in a microwave reactor and stirred for 10 minutes. The mixture was heated to 150° C. and stirred for a further 10 minutes. The mixture was cooled to room temperature and concentrated under vacuum. The residue was suspended in dilute hydrochloric acid and filtered. The filter-cake was washed with water and cyclohexane then dried under vacuum to give the title compound as a dark grey solid (0.95 g, 78%). 1H-NMR (400 MHz, DMSO-d6): δ 10.73 (1H, br s), 10.62 (1H, br s), 6.87 (1H, dd, J 8.5, 5 Hz), 6.78-6.70 (2H, m). UPLC-MS: 0.47 min, m/z 153 [M+H]+.

### Intermediate 16: 3-(3,4-Dichlorophenyl)-1-oxa-3,8-diazaspiro[4.5]decan-2-one

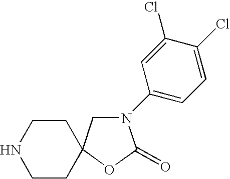

To a suspension of phenylmethyl 3-(3,4-dichlorophenyl)-2-oxo-1-oxa-3,8-diazaspiro[4.5]decane-8-carboxylate (Intermediate 17, 29.58 g, 68 mmol) in industrial methylated spirits (250 ml) was added palladium on carbon (10%, 5 g). The mixture was hydrogenated for 6 hours at 50 p.s.i. The mixture was filtered through a celite pad, washing with hot methanol (3×500 ml). The combined filtrates were concentrated under vacuum.

The above procedure was repeated; the 2 batches of crude product were combined with another product batch prepared in an analogous manner using phenylmethyl 3-(3,4-dichlorophenyl)-2-oxo-1-oxa-3,8-diazaspiro[4.5]decane-8-carboxylate (2.15 g), industrial methylated spirits (40 ml) and palladium on carbon (10%, 350 mg). The combined batches were recrystallised from methanol to give the title compound (21.97 g). 1H-NMR (400 MHz, DMSO-d6): δ 1.80-1.95 (4H, m), 2.80-3.00 (4H, m), 3.90 (2H, s), 7.55-7.62 (1H, m), 7.63-7.70 (1H, m) and 7.85 (1H, d); MS: m/z 342, [M+MeCN]+.

The title compound may be converted in its hydrobromide salt by methods known to the skilled person. The hydrobromide salt has the following analytical data: 1H-NMR (400 MHz, DMSO-d6): δ 2.00-2.20 (4H, m), 3.10-3.32 (4H, m), 4.00 (2H, s), 7.58 (1H, dd), 7.68 (1H, d), 7.82 (1H, d), 8.65 (2H, br s); MS: m/z 301 and 303, [M+H]+.

### Intermediate 17: Phenylmethyl3-(3,4-dichlorophenyl)-2-oxo-1-oxa-3,8-diazaspiro[4.5]decane-8-carboxylate

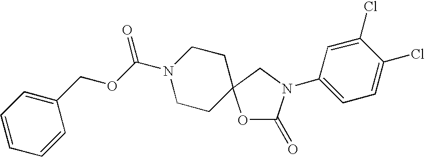

To a stirred solution of phenylmethyl 4-{[(3,4-dichlorophenyl)amino]methyl}-4-hydroxy-1-piperidinecarboxylate (Intermediate 18, 74.9 g, 188 mmol) in dichloromethane (900 ml) was added triethylamine (52.3 ml, 376 mmol). The mixture was cooled to 5° C. (ice bath). To this mixture was added a solution of triphosgene (28.215 g, 95 mmol) in dichloromethane (100 ml) over 20 minutes. The mixture was stirred 1 hour 30 min at r.t., then cooled to 5° C. (ice bath). To the mixture was added aqueous sodium carbonate solution (10%, 250 ml). The organic phase was separated and the aqueous was extracted with DCM (3×50 ml). The combined organic layers were dried over anhydrous magnesium sulfate and concentrated under vacuum to give an orange-brown oil (87.2 g), which crystallised on standing for 48 hours. EtOAc (250 ml) was added, and the resultant mixture was heated gently while agitating with a spatula. To the mixture was added hexane (700 ml); the resultant precipitate was filtered off, washed with pentane (2×100 ml) and dried overnight at 50° C. on a hot plate to give the title compound as a yellowish solid (64.44 g, 78%); Rf 0.36 (1:1 EtOAc-hexane, black spot with phosphomolybdic acid); m.p. 105° C.

### Intermediate 18: Phenylmethyl4-{[(3,4-dichlorophenyl)amino]methyl}-4-hydroxy-1-piperidinecarboxylate

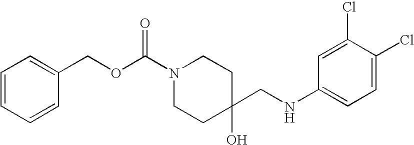

A mixture of phenylmethyl 1-oxa-6-azaspiro[2.5]octane-6-carboxylate (for a preparation see e.g. U.S. Pat. No. 4,244,961, 46.6 g, 0.188 mol), 3,4-dichloroaniline (213.4 g, 1.316 mol, Aldrich) and ethoxyethanol (300 ml) was heated under reflux overnight. The mixture was concentrated under vacuum to give the title compound (74.9 g), which was used without further purification; Rf 0.28 (1:1 EtOAc-hexane); m.p. 128-130° C.

2-Chloro-5-(trifluoromethyl)-1H-benzimidazole and 2,5-dichloro-1H-benzimidazole are commercially available from Prime Organics, alternatively 2-chloro-5-(trifluoromethyl)-1H-benzimidazole can be prepared according to the description above (Intermediate 1). 2-Chloro-5-(methyloxy)-1H-benzimidazole and 2-chloro-1H-benzimidazole are commercially available from suppliers including Lancaster.

### Intermediate 19: 1,1-Dimethylethyl 1-oxa-6-azaspiro[2,5]octane-6-carboxylate

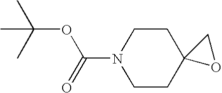

To a mixture of sodium hydride (54 g, 1 mol) and dry DMSO (1 I), trimethyl sulfoxonium iodide (220 g, 1 mol) was added in 5 g lots and stirred for 2 hours. Then a solution of N-Boc piperidone (commercially available, 200 g, 1.0 mol) in DMSO (1000 ml) was added over a period of 2 hours (exothermic, temperature went up to 40° C.) and stirred at 55° C. for 2 hours. The reaction mixture was quenched with ice-water and extracted with diethyl ether (2×2 I). The combined organic layer was washed with water, brine, dried over sodium sulphate and concentrated. The crude product (viscous brown oil) was taken to next step without further purification. Crude yield: 200 g (93%).

### Intermediate 20: 3-Methyl-1-oxa-3,8-diazaspiro[4.5]decan-2-one hydrochloride

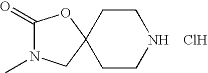

1,1-Dimethylethyl 3-methyl-2-oxo-1-oxa-3,8-diazaspiro[4.5]decane-8-carboxylate (Intermediate 21, 48 g) was dissolved in dry methanol (100 ml). To this, HCl in methanol (150 ml) was added and the mixture was stirred at r.t. for 30 mins. The reaction mixture was concentrated under reduced pressure, and resulting solid was recrystallised by dissolving in dry methanol (100 ml), adding EtOAc (250 ml) and petroleum ether (250 ml). The solid was collected by filtration and washed with petroleum ether. The recrystallisation, filtration and washing process were repeated to give the desired product (28.5 g); LC-MS (4.5 min): 0.22 min, m/z 171.2 [M+H]+.

### Intermediate 21: 1,1-Dimethylethyl 3-methyl-2-oxo-1-oxa-3,8-diazaspiro[4.5]decane-8-carboxylate

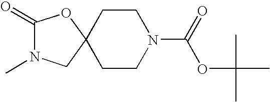

1,1-Dimethylethyl-4-hydroxy-4-[(methylamino)methyl]-1-piperidinecarboxylate (Intermediate 22, 55 g, 0.225 mol) was dissolved in dry dichloromethane (500 ml). To this was added triethylamine (188 ml) and the mixture was cooled to 0° C. A solution of triphosgene (26.7 g) in dry DCM (500 ml) was added slowly at 0° C. with stirring. The reaction mixture was then stirred at room temperature for 30 min. Water was added (100 ml) and the mixture was stirred for 30 mins. The aqueous layer was separated and the dichloromethane layer was washed with water (2×500 ml) and saturated brine solution (2×500 ml), dried over sodium sulphate and concentrated. The crude product was recrystallised by dissolving in diethyl ether (70 ml) and added petroleum ether (350 ml). The solid was filtered and washed with petroleum ether to give the desired product (48 g); TLC (CHCl₃: MeOH 8:2) Rf 0.63.

### Intermediate 22: 1,1-Dimethylethyl 4-hydroxy-4-[(methylamino)methyl]-1-piperidine-carboxylate

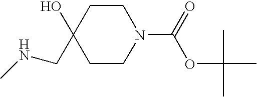

1,1-Dimethylethyl1-oxa-6-azaspiro[2.5]octane-6-carboxylate (prepared in an analogous manner to Intermediate 19, 160 g) was dissolved in methanol (400 ml) and cooled to 0° C. At this temperature methylamine (25% solution in methanol, 400 ml) was added slowly. After addition, the resulting mixture was stirred at r.t. overnight then concentrated under reduced pressure. The crude material was loaded onto a column; the impurity was removed by washing with petroleum ether, and product was eluted by adding ethyl acetate. The product-containing fractions were concentrated under reduced pressure and the resulting gummy solid was dried under high vacuum to give the title compound (120 g); TLC (CHCl₃:MeOH 9:1) Rf 0.1.

### Intermediate 23: 2-chloro-5-(trifluoromethoxy)-1H-benzimidazole

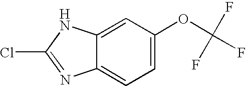

5-[(Trifluoromethyl)oxy]-1,3-dihydro-2H-benzimidazol-2-one (Intermediate 24, 1.1 g, 5.0 mmol) in phosphorus oxychloride (10 ml) was stirred at 130° C. for 2 hours and cooled. The mixture was added carefully to aqueous potassium carbonate solution and extracted into ethyl acetate (400 ml). The organic layer was dried over magnesium sulphate, diluted with hexane (400 ml) and filtered through a silica plug. The filtrate was evaporated; the solid residue was washed with ether/hexane (1:9) and dried to give title compound (0.57 g, 48%); LCMS: 3.08 min, m/z 237.0 [M+H]+.

### Intermediate 24: 5-[(trifluoromethyl)oxy]-1,3-dihydro-2H-benzimidazol-2-one

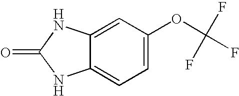

4-[(Trifluoromethyl)oxy]-1,2-benzenediamine (commercially available, 1.03 g, 5.4 mmol) and urea (0.6 g, 10 mmol) in DMF were heated to 150° C. overnight and added to water (100 ml). The light brown solid precipitate was filtered off, washed with water and dried to give the desired product (1.1 g, 94%) which was used without further purification in the preparation of Intermediate 23.

### Intermediate 25: N-[2-Amino-5-(trifluoromethyl)phenyl]-3-oxo-2-phenyl-2-azaspiro[4.5]-decane-8-carboxamide

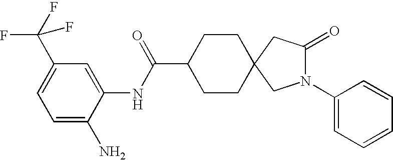

To a stirred solution of 3-oxo-2-phenyl-2-azaspiro[4.5]decane-8-carboxylic acid (Intermediate 26, 0.11 g, 0.4 mmol) and 4-trifluoromethyl-1,2-diaminobenzene (commercially available, 106 mg, 1.5 eq) in pyridine (2 ml) was added EDAC (0.12 g, 1.6 eq.). The mixture was stirred 1 hour, then left to stand 18 hours. The mixture was concentrated under vacuum then partitioned between aqueous sodium bicarbonate solution and ethyl acetate (×2). The combined organic extracts were washed with water, filtered through a hydrophobic membrane (Phase Sep) and concentrated under vacuum to give the crude product (0.217 g). The crude was purified by chromatography on silica gel eluting with cyclohexane-ethyl acetate (4:1 to 0:1 gradient) to give the title compound (0.14 g, 80%) as a viscous yellow oil (5:1 mixture of trans:cis isomers). 1H-NMR (400 MHz, CDCl₃): δ 8.30-8.15 (1H, m), 7.53 (2H, d, J 7.5 Hz), 7.40-7.31 (3H, m), 7.29-7.20 (1H, m), 7.14 (1H, t, J 7.5 Hz), 6.98-6.93 (0.17H, m), 6.72 (0.83H, d, J 8.5 Hz), 4.23 (2H, m), 3.64 (0.34H, s), 3.53 (1.66H, s), 2.45 (1.66H, s), 2.37 (0.34H, s), 2.29 (1H, m), 1.90-1.74 (4H, m), 1.71-1.56 (2H, m) and 1.43-1.32 (2H, m); UPLC-MS: 0.71 min, m/z 432 [M+H]+ (isomers not resolved).

### Intermediate 26: 3-Oxo-2-phenyl-2-azaspiro[4.5]decane-8-carboxylic acid

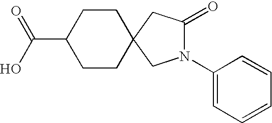

To a stirred solution of ethyl 3-oxo-2-phenyl-2-azaspiro[4.5]decane-8-carboxylate (Intermediate 27, 0.21 g, 0.70 mmol) in methanol (2 ml) was added dropwise aqueous sodium hydroxide solution (0.5M, 1.7 ml, 1.2 eq.). The mixture was stirred 2 hours then left to stand 18 hours. The mixture was partitioned between water and cyclohexane. The aqueous layer was acidified and extracted twice with ethyl acetate. The combined ethyl acetate extracts were washed with water, filtered through a hydrophobic membrane (Phase Sep) and concentrated under vacuum to give the title compound as a white solid (199 mg), which was used without further purification. 1H-NMR (400 MHz, CDCl₃): δ 7.63-7.57 (2H, m), 7.41-7.35 (2H, m), 7.19-7.13 (1H, m), 3.69 (0.34H, s), 3.61 (1.64H, s), 2.55 (1.66H, s), 2.49 (0.34H, s), 2.41 (1H, m), 2.04-1.93 (1H, m), 1.92-1.84 (1H, m), 1.74-1.61 (2H, m) and 1.58-1.47 (2H, m); UPLC-MS: 0.58 and 0.62 min, both m/z 274 [M+H]+. 5:1 mixture of trans:cis isomers.

### Intermediate 27: Ethyl 3-oxo-2-phenyl-2-azaspiro[4.5]decane-8-carboxylate

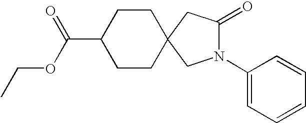

A mixture of ethyl 3-oxo-2-azaspiro[4.5]decane-8-carboxylate (Intermediate 28, 0.20 g, 0.89 mmol), iodobenzene (0.10 ml, 1 eq.), copper(I) iodide (8 mg, 5 mol %), trans-1,2-diaminocyclohexane (11 μl, 10 mol %), cesium carbonate (0.58 g, 2 eq.) and toluene (1 ml) was heated to 80° C. in a closed vial under nitrogen and stirred vigorously for 18 hours. The mixture was cooled to room temperature and partitioned between water and ethyl acetate (×2). The combined organic extracts were washed with dilute hydrochloric acid, then water, then brine, filtered through a hydrophobic membrane (Phase Sep) and concentrated under vacuum to give the crude product (0.265 g) as a yellow oil, which crystallised on standing. The crude was purified by column chromatography on silica gel, eluting with 4:1 cyclohexane ethyl acetate to give the title compound as a white solid (0.229 g, 85%, 5.5:1 mixture of trans:cis isomers). 1H-NMR (400 MHz, CDCl₃): δ 7.63-7.58 (2H, m), 7.40-7.34 (2H, m), 7.18-7.13 (1H, m), 4.15 (2H, q, J 7 Hz), 3.68 (0.3H, s), 3.60 (1.7H, s), 2.54 (1.7H, s), 2.47 (0.3H, s), 2.39-2.29 (1H, m), 1.99-1.91 (2H, m), 1.89-1.82 (2H, m), 1.70-1.58 (2H, m), 1.55-1.43 (2H, m) and 1.27 (3H, t, J 7 Hz); UPLC-MS: 0.74 and 0.75 min, both m/z 302 [M+H]+.

### Intermediate 28: Ethyl3-oxo-2-azaspiro[4,5]decane-8-carboxylate

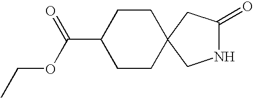

To a stirred mixture of iron powder (0.92 g), ammonium chloride (1.1 g, 5 eq.), and water (2 ml) was added dropwise a solution of ethyl 4-[2-(ethyloxy)-2-oxoethyl]-4-(nitromethyl)cyclohexanecarboxylate (Intermediate 29, 4.1 mmol) in ethanol (5 ml). The resultant mixture was heated to 70° C. and stirred for 90 minutes, then heated to 80° C. and stirred 45 minutes. The mixture was cooled to r.t., left to stand 72 hours then filtered, washing with ethanol. The filtrate was concentrated under vacuum. The residue was partitioned between dilute aqueous sodium hydroxide solution and ethyl acetate (×2). The combined organic layers were washed twice with water and then with brine, filtered through a hydrophobic membrane (Phase Sep) and concentrated under vacuum to give the crude product as an oil, which crystallised on standing (0.77 g). The crude was purified by chromatography on silica gel, eluting with ethyl acetate to give the title compound (0.55 g, 59%, 5.8:1 trans:cis isomers mixture). 1H-NMR (400 MHz, CDCl₃): δ 6.04 (0.85H, m, NH), 5.96 (0.15H, m, NH), 4.13 (2H, q, J 7 Hz), 3.22 (0.3H, s), 3.13 (1.7H, s), 2.34-2.24 (1H, m), 2.23 (1.7H, s), 2.16 (0.3H, s), 1.94-1.85 (2H, m), 1.84-1.76 (2H, m), 1.64-1.51 (2H, m), 1.48-1.36 (2H, m), 1.26 (3H, t, J 7 Hz); HPLC-MS, 1: 1.729 min, m/z 226 [M+H]+ and 451 [2M+H]+.

### Intermediate 29: Ethyl4-[2-(ethyloxy)-2-oxoethyl]-4-(nitromethyl)cyclohexanecarboxylate

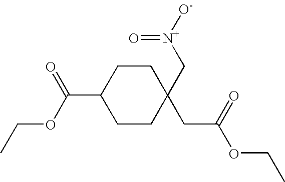

A mixture of ethyl 4-[2-(ethyloxy)-2-oxoethylidene]cyclohexanecarboxylate (Intermediate 30, 1.3 g, 5.4 mmol), nitromethane (1.15 ml, 4 eq.), tetrabutylammonium fluoride trihydrate (2.8 g, 2 eq.) and THF (20 ml) was heated to 40° C. and stirred 24 hours. The mixture was cooled to room temperature and partitioned between aqueous ammonium chloride solution and ethyl acetate (×2). The combined organic extracts were washed with brine, filtered through a hydrophobic membrane (Phase Sep) and concentrated under vacuum to give the crude product as a brown oil (2.19 g). The crude was purified by silica gel chromatography, eluting with cyclohexane-ethyl acetate (1:0 to 6:1) to give the title compound (1.28 g, 79%, 5.7:1 mixture of trans:cis isomers). 1H-NMR (400 MHz, CDCl₃): δ 4.80 (0.3H, s), 4.64 (1.7H, s), 4.19-4.11 (4H, m), 2.61 (1.7H, s), 2.48 (0.3H, s), 2.38-2.26 (1H, m), 1.93-1.80 (4H, m), 1.76-1.62 (2H, m), 1.47-1.36 (2H, m) and 1.31-1.24 (6H, m); UPLC-MS: 0.77 min, m/z 302 [M+H]+.

### Intermediate 30: Ethyl4-[2-(ethyloxy)-2-oxoethylidene]cyclohexanecarboxylate

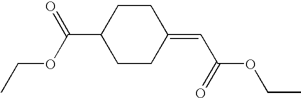

To a stirred suspension of sodium hydride (60%, 0.94 g, 23.5 mmol) in THF at 0° C. was added dropwise triethyl phosphonoacetate (4.66 ml, 23.5 mmol). The mixture was stirred 30 minutes then a solution of ethyl 4-oxocyclohexanecarboxylate (2.0 g, 11.8 mmol) in THF (10 ml) was added dropwise. The mixture was warmed to r.t. and stirred for 18 hours. The mixture was quenched with aqueous ammonium chloride solution and extracted twice with ethyl acetate. The combined organic extracts were washed with water, then brine, filtered through a hydrophobic membrane (Phase Sep) and concentrated under vacuum to give the crude product (3.98 g) as a clear oil. The crude was purified by silica gel chromatography, eluting with cyclohexane-ethyl acetate (1:0 to 4:1) to give the title compound (1.30 g, 46%) plus a mixture of the title compound with mineral oil (1.84 g). 1H-NMR (400 MHz, CDCl₃): δ 5.65 (1H, br s), 4.18-4.11 (4H, m), 3.62 (1H, dt, J 14, 4.5 Hz), 2.59-2.51 (1H, m), 2.35 (1H, dt, J 14, 4.5 Hz), 2.25-2.15 (2H, m), 2.10-2.02 (2H, m), 1.77-1.63 (2H, m), 1.30-1.23 (6H, m); UPLC-MS: 0.80 min, m/z 241 [M+H]+ and 195 [M-OEt]+.

### Intermediate 31: (Cis)-2-oxo-3-phenyl-1-oxa-3-azaspiro[4.5]decane-8-carbaldehyde

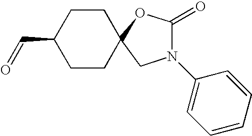

(Trans)-8-(hydroxymethyl)-3-phenyl-1-oxa-3-azaspiro[4.5]decan-2-one (Intermediate 33, 0.262 g, 1.003 mmol) was dissolved in DCM (20 ml). Tetra-n-propylammonium perruthenate(VII) (0.045 g, 0.127 mmol) and N-methylmorpholine N-oxide (0.176 g, 1.504 mmol) were added and the mixture was stirred at room temperature under a nitrogen atmosphere for 1.5 hour. The mixture was filtered through a celite pad washing with DCM. The filtrate was dried to give a residue that was purified by column chromatography (Silica 25+M, Biotage SP1) eluting with cyclohexane:EtOAc from 9:1 to 5:5 then washed with 100% EtOAc to afford the title compound as a white solid (0.051 g, 17%, lower running fraction) and the corresponding upper running fraction trans isomer (0.071 g, 25%). 1H-NMR (400 MHz, CDCl₃): δ 1.60-1.73 (m, 2H), 1.88-2.02 (m, 4H), 2.15-2.36 (m, 3H), 3.77 (s, 2H), 7.13-7.19 (m, 1H) 7.36-7.43 (m, 2H) 7.52-7.58 (m, 2H) 9.65-9.68 (s, 1H); HPLC-MS, 1: 2.249 min, m/z 260 [M+H]+.

4-(Trifluoromethoxy)-1,2-benzenediamine is commercially available, alternatively it can be prepared according to the following procedure.

### Intermediate 32: 4-(Trifluoromethoxy)-1,2-benzenediamine

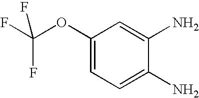

To a stirred mixture of iron (2.011 g, 36.0 mmol), ammonium chloride (2.408 g, 45.0 mmol) and water (5 ml) was added dropwise a solution of 2-nitro-4-(trifluoromethoxy)aniline (commercially available from e.g. Fluorochem. Ltd., 2 g, 9.0 mmol) in ethanol (10 ml). The mixture was heated to 70° C. and stirred for 4 hours. By TLC the reaction was complete. The mixture was cooled to room temperature and filtered through celite, washing with ethanol. The filtrate was concentrated under vacuum; the residue was partitioned between dichloromethane and water. The organic phase was filtered through a hydrophobic membrane (Phase Sep) and concentrated under vacuum to give the title compound (1.51 g) as a dark brown oil. 1H-NMR (400 MHz, CDCl₃): δ 6.66 (1H, d, J 8 Hz), 6.60-6.55 (2H, m), 3.84-2.89 (4H, m); UPLC-MS: 0.50 min, m\z 193 [M+H]+.

### Intermediate 33: (Trans)-8-(hydroxymethyl)-3-phenyl-1-oxa-3-azaspiro[4.5]decan-2-one

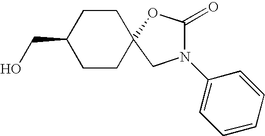

Ethyl(trans)-2-oxo-3-phenyl-1-oxa-3-azaspiro[4.5]decane-8-carboxylate (Intermediate 11, 0.611 g, 2.044 mmol) was dissolved in dry tetrahydrofuran (20 ml) under a nitrogen atmosphere. The mixture was cooled to −78° C. and lithium aluminium hydride (1M solution in THF, 1.533 ml, 1.533 mmol) was slowly added. The mixture was left at −78° C. for 1.5 hour. MS check showed some starting material left, then a further 0.6 ml of 1M lithium aluminium hydride solution was added and the mixture was stirred at −78° C. for another 1.5 hours. Then two spatulae of Na₂SO₄ decahydrate were slowly added and the mixture was stirred for 2 hours then left to stand overnight while the temperature slowly rose to room temperature. The precipitate was filtered off washing with Et₂O and the filtrate was dried (rotary evaporator) to give 580 mg of crude product that was purified by column chromatography (Silica 25+M, Biotage SP1) eluting with cyclohexane:EtOAc 9:1 to 1:1 to afford the title compound (0.265 g, 49%) and the corresponding aldehyde (0.091 g, 17%). 1H-NMR (400 MHz, CDCl₃): δ 1.10-1.29 (m, 2H), 1.29-1.39 (t, 1H), 1.60-1.67 (m, 1H), 1.82-1.93 (m, 2H), 1.92-2.06 (m, 4H), 3.56 (t, 2H), 3.80 (m, 2H), 7.06-7.23 (m, 1H), 7.34-7.45 (m, 2H), 7.51-7.66 (m, 2H); UPLC-MS: 0.60 min, m/z 262 [M+H]+.

### Intermediate 34: Mixture of (trans)-N-{2-amino-4-[trifluoromethyl)oxy]phenyl}-2-oxo-3-phenyl-1-oxa-3-azaspiro[4.5]decane-8-carboxamide and (trans)-N-{2-amino-5-[(trifluoromethyl)oxy]phenyl}-2-oxo-3-phenyl-1-oxa-3-azaspiro[4.5]decane-8-carboxamide,

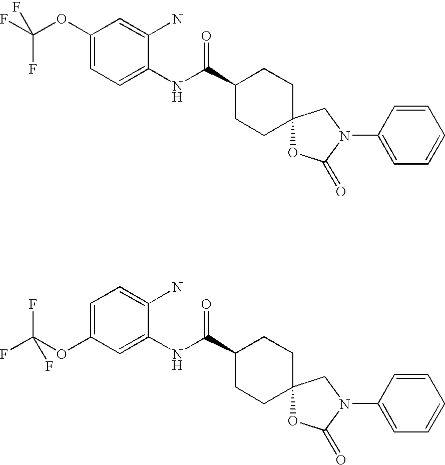

(Trans)-2-oxo-3-phenyl-1-oxa-3-azaspiro[4.5]decane-8-carboxylic acid (Intermediate 10, 90 mg, 0.327 mmol), 4-[(trifluoromethyl)oxy]-1,2-benzenediamine (Intermediate 32, 94 mg, 0.490 mmol) and EDC (100 mg, 0.52 mmol), were suspended in pyridine (2 ml) and shaken for 18 hours. The solvent was then removed under vacuum; the residue was dissolved in DCM (5 ml), which was washed with aqueous NaHCO₃ solution, concentrated under vacuum and then purified on Biotage SP1, 25+M silica cartridge, using a gradient of cyclohexane and ethyl acetate as eluent. The title compound was eluted with 50% ethyl acetate, and recovered as brown solid (130 mg) as a mixture of two regioisomers (unknown ratio). 1H-NMR (400 MHz, CDCl₃): δ 1.8-2.2 (m), 3.8 (s), 6.6 (m), 7.1-7.45 (m), 7.55 (m); UPLC-MS: 0.75 min, 450 [M+H]+.

### Intermediate 35: 1:3 Mixture of (trans)-N-{2-amino-4-[(trifluoromethyl)oxy]phenyl}-3-(2-fluorophenyl)-2-oxo-1-oxa-3-azaspiro[4.5]decane-8-carboxamide and (trans)-N-{2-amino-5-[trifluoromethyl)oxy]phenyl}-3-(6-methyl-2-pyridinyl)-2-oxo-1-oxa-3-azaspiro[4.5]decane-8-carboxamide

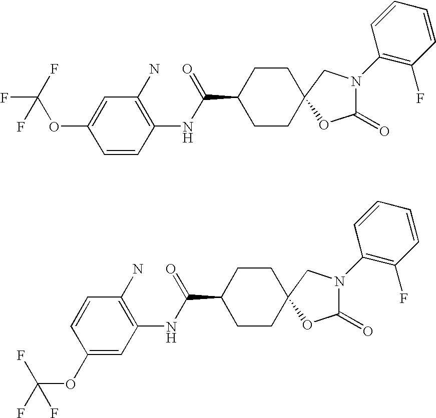

To a mixture of (trans)-3-(2-fluorophenyl)-2-oxo-1-oxa-3-azaspiro[4.5]decane-8-carboxylic acid (Intermediate 36, 96 mg, 0.327 mmol) and 4-[(trifluoromethyl)oxy]-1,2-benzenediamine (Intermediate 32, 94 mg, 0.491 mmol) in pyridine (1.5 mL) was added EDC.HCl (125 mg, 0.655 mmol) and the mixture was shaken at room temperature for 1.5 hour. The reaction mixture was concentrated under vacuum and the residue partitioned between saturated aqueous sodium bicarbonate solution and EtOAc (2×10 ml). The combined organic extracts were washed with brine, dried (Na₂SO₄) and concentrated under vacuum to give a residue. The residue was purified by silica gel chromatography eluting with a gradient from 0-60% EtOAc\cyclohexane to obtain an oil which was in turn purified by silica gel chromatography (eluent 5% Et₂O\DCM) to give the title compound as a solid (139 mg, 86%). 1H-NMR (400 MHz, CDCl₃): δ 1.5-1.85 (m, 8H), 1.9-2.15 (m, 8H), 3.8 (s, 4H), 5.1-5.3 (2×s, together 4H), 6.4-6.9 (m, together 4H), 7.2-7.45 (m, 8H), 7.6 (t, 2H), 9.1-9.2 (2×s, together 2H); UPLC-MS: 0.73 min, 468 [M+H]+.

### Intermediate 36: (Trans)-3-(2-fluorophenyl)-2-oxo-1-oxa-3-azaspiro[4.5]decane-8-carboxylic acid

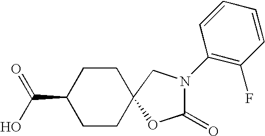

To a stirred solution of ethyl(trans)-3-(2-fluorophenyl)-2-oxo-1-oxa-3-azaspiro[4.5]decane-8-carboxylate (Intermediate 37, 119 mg, 0.370 mmol) in MeOH (4 ml) was added dropwise a solution of lithium hydroxide (44.3 mg, 1.852 mmol) in water (1 ml) and the mixture was stirred at room temperature for 1 hour. The mixture was acidified to pH 1.0 with 1N HCl, concentrated under vacuum, diluted with water and extracted with EtOAc. The combined organic extracts were washed with brine, dried (Na₂SO₄), filtered and concentrated under vacuum to obtain the title compound as white crystalline solid (101 mg, 93%). UPLC-MS: 0.57 min, 294 [M+H]+.

### Intermediate 37: Ethyl(trans)-3-(2-fluorophenyl)-2-oxo-1-oxa-3-azaspiro[4.5]decane-8-carboxylate

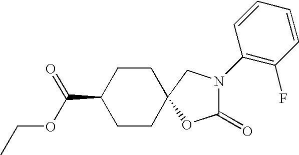

To a stirred solution of 1-fluoro-2-iodobenzene (62 μl, 0.533 mmol) and ethyl(trans)-2-oxo-1-oxa-3-azaspiro[4.5]decane-8-carboxylate (Intermediate 38, 101 mg, 0.444 mmol) in anhydrous 1,4-dioxane (1.5 ml), under a nitrogen atmosphere were added copper(I) iodide (4.23 mg, 0.022 mmol), trans-cyclohexanediamine (5.36 μl, 0.044 mmol) and potassium phosphate tribasic (189 mg, 0.889 mmol). The mixture was stirred at 110° C. for 2 hours, then diluted with ethyl acetate and washed with water. The organic extracts were dried (Na₂SO₄), filtered and concentrated under vacuum to give a residue. The residue was purified by silica gel chromatography eluting with 100% cyclohexane, then 20% EtOAc\cyclohexane. to give the title compound as a colourless oil (119 mg, 82%). 1H NMR (400 MHz, CDCl₃): δ 1.28 (t, 3H), 1.67-1.81 (m, 2H), 1.85-2.06 (m, 4H), 2.06-2.17 (m, 2H), 2.45-2.57 (m, 1H), 3.78-3.83 (s, 2H), 4.16 (q, 2H), 7.10-7.30 (m, 3H), 7.55 (m, 1H); UPLC-MS: 0.73 min, 322 [M+H]+.

### Intermediate 38 and 39: (Trans) ethyl 2-oxo-1-oxa-3-azaspiro[4.5]decane-8-carboxylate (Intermediate 38) and (cis) ethyl 2-oxo-1-oxa-3-azaspiro[4.5]decane-8-carboxylate (Intermediate 39)

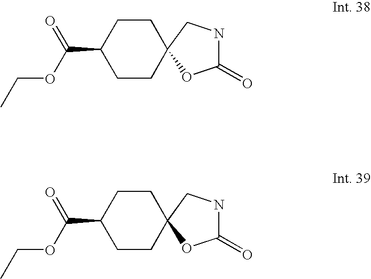

5.5 g of a mixture of diastereomeric ethyl 2-oxo-1-oxa-3-azaspiro[4.5]decane-8-carboxylate (Intermediate 40) were submitted to silica gel chromatography eluting with cyclohexane/EtOAc 1:1 to pure EtOAc. 3.73 g of the trans isomer (first title compound, Intermediate 38) were collected whereas 3.62 g of cis isomer (Intermediate 39) were obtained.

Trans isomer (Intermediate 38): 1H-NMR (400 MHz, CDCl₃): δ 5.40 (1H, br s), 4.16 (2H, q), 3.37 (2H, s), 2.47 (1H, sept), 2.01-2.12 (2H, m), 1.79-1.96 (4H, m), 1.63-1.74 (2H, m), 1.27 (3H, t); UPLC-MS: 0.53 min, 228 [M+H]+, 455 [2M+H]+.

Cis isomer (Intermediate 39): 1H-NMR (400 MHz, CDCl₃): δ 5.33 (1H, br s), 4.15 (2H, q), 3.33 (2H, s), 2.27-2.38 (1H, m), 2.08-2.18 (2H, m), 1.86-2.04 (4H, m), 1.47-1.59 (2H, m), 1.28 (3H, t); UPLC-MS: 0.52 min, 228 [M+H]+, 455 [2M+H]+.

### Intermediate 40: Ethyl 2-oxo-1-oxa-3-azaspiro[4.5]decane-8-carboxylate

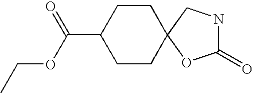

Ethyl carbamate (15.60 g, 175 mmol) was dissolved in DMPU (107 ml). After cooling to 0° C. potassium tert-butoxide (13.10 g, 117 mmol) was added in portions over 5 min. The cooling bath was removed and the reaction mixture was stirred for 1 hour at r.t. Ethyl 1-oxaspiro[2.5]octane-6-carboxylate (Intermediate 8, prepared according to the third method above described, 10.75 g, 58.4 mmol) dissolved in DMPU (20 ml) was added and the mixture heated at 130° C. for 15 hours. The mixture was allowed to cool to r.t. and quenched with sat. aq. NH₄Cl (60 ml) while cooling by means of an ice-bath. The bath was removed and further sat. aq. NH₄Cl (300 ml) was added until a clear solution was obtained, which was diluted further with brine (300 ml). The aqueous solution was extracted with DCM (3×400 ml). The combined organics were dried (Na₂SO₄), filtered and the DCM evaporated under reduced pressure. The remaining DMPU was stripped off under reduced pressure (80° C., 0.1 Torr) over 7 hours. The obtained solid was purified by silica gel chromatography eluting with DCM\EtOAc: 8/2. 10 g of the title compound was collected (44.0 mmol, 75%) along with a second slightly impure batch of 2 g (8.36 mmol; 14.3%) of the title compound. 1H-NMR (400 MHz, CDCl₃): δ 5.07-5.22 (1H, m), 4.15 (2H, q), 3.37 (0.6H, s), 3.32 (0.4H, s), 2.42-2.53 (0.4H, m), 2.26-2.39 (0.6H, m), 2.01-2.18 (2H, m), 1.79-2.01 (4H, m), 1.63-1.75 (0.6H, m), 1.46-1.58 (1.4H, m), 1.27 (3H, t) (6:4 mixture of cis and trans isomers).

### Intermediate 41: Mixture of (trans)N-{2-amino-4-[(trifluoromethyl)oxy]phenyl}-3-(4-fluorophenyl)-2-oxo-1-oxa-3-azaspiro[4.5]decane-8-carboxamide and (trans)N-{2-amino-5-[(trifluoromethyl)oxy]phenyl}-3-(4-fluorophenyl)-2-oxo-1-oxa-3-azaspiro[4.5]decane-8-carboxamide

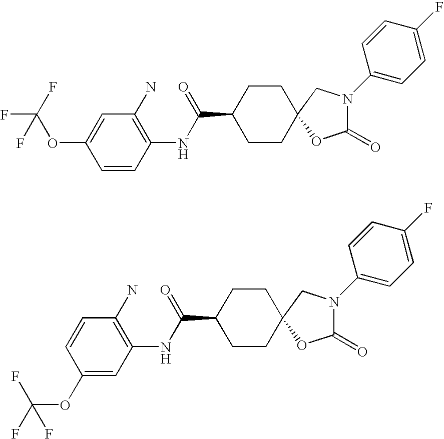

4-[(Trifluoromethyl)oxy]-1,2-benzenediamine (Intermediate 32, 177 mg, 0.921 mmol) and (trans)3-(4-fluorophenyl)-2-oxo-1-oxa-3-azaspiro[4.5]decane-8-carboxylic acid (Intermediate 42, 180 mg, 0.614 mmol) were dissolved in pyridine (2.5 ml) and EDC (188 mg, 0.982 mmol) was added. The mixture was stirred at r.t. for 2 hours. The pyridine was removed under vacuum and the residue partioned between aq. sat. NaHCO₃ (20 ml) and EtOAc (50 ml). The aqueous layer was extracted once again with EtOAc (30 ml). The combined EtOAc extracts were washed with brine (20 ml), dried (Na₂SO₄), filtered and evaporated. The residue was purified by silica gel chromatography eluting with cyclohexane\EtOAc 6\4 to 4\6. The title compounds were obtained as a mixture of both possible regioisomers and were collected together to give 274.5 mg (0.529 mmol, 86%). 1H-NMR (400 MHz, CDCl₃): (data given for major isomer) δ 7.54 (2H, dd), 7.18-7.25 (1H, m), 7.19 (2H, t), 6.64-6.73 (1H, m), 3.77 (2H, s), 2.48-2.58 (1H, m), 2.10-2.23 (4H, m), 1.80-2.00 (4H, m), one aromatic proton not detected, might fall underneath CHCl₃ signal; UPLC-MS: 0.75 min, 468 [M+H]+.

### Intermediate 42: (Trans)3-(4-fluorophenyl)-2-oxo-1-oxa-3-azaspiro[4.5]decane-8-carboxylic acid

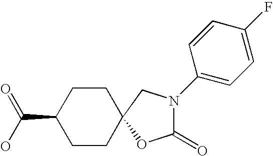

(Trans) ethyl 3-(4-fluorophenyl)-2-oxo-1-oxa-3-azaspiro[4.5]decane-8-carboxylate (Intermediate 43, 202 mg, 0.629 mmol) was dissolved in methanol (4.2 ml). Lithium hydroxide (75.2 mg, 3.14 mmol) was added dropwise as a solution in water (0.84 ml). The mixture was stirred for 1 hour at r.t. The mixture was brought to pH 2 with 1M hydrochloric acid and extracted with EtOAc (3×20 ml). The combined organics were washed with water, dried (Na₂SO₄), filtered and evaporated to give 182 mg (0.621 mmol, 99%) of the title compound, which was sufficiently pure to be used in the next step. 1H-NMR (400 MHz, CDCl₃): δ 7.52 (2H, dd), 7.09 (2H, t), 3.77 (2H, s), 2.64 (1H, sept), 2.11-2.23 (2H, m), 1.89-2.03 (4H, m), 1.78-1.89 (2H, m); UPLC-MS: 0.61 min, 292 [M−H]−, 585 [2M−H]−.

### Intermediate 43: Ethyl(trans)-3-(4-fluorophenyl)-2-oxo-1-oxa-3-azaspiro[4.5]decane-8-carboxylate

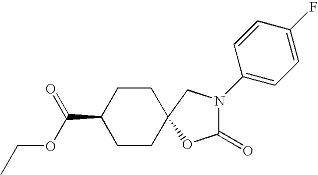

1,2-Cyclohexanediamine (0.037 ml, 0.308 mmol), ethyl(trans)-2-oxo-1-oxa-3-azaspiro[4.5]decane-8-carboxylate (Intermediate 38, 700 mg, 3.08 mmol), cesium carbonate (2.509 g, 7.70 mmol) and copper(I) iodide (29.3 mg, 0.154 mmol) were heated in toluene (5.3 ml) at 80° C. for 12 hours under nitrogen atmosphere and vigorous stirring. After this time, the reaction had only progressed a little. The mixture was transferred into a microwave vial, capped and heated at 80° C. for 7 hours. All starting material was consumed. The reaction mixture was cooled to r.t., diluted with EtOAc (50 ml) and washed with water (20 ml). The aqueous phase was extracted with EtOAc (30 ml). The combined organics were washed with 1M HCl (20 ml) and water (20 ml), then dried (Na₂SO₄), filtered and evaporated. The residue was purified by silica gel chromatography eluting with cyclohexane\EtOAc 9:1 to 1:1 to give 227 mg (0.706 mmol, 22.9%) of the title compound and 217 mg (0.675 mmol, 21.9%) of its cis isomer.

Trans isomer (Intermediate 43): 1H-NMR (400 MHz, CDCl₃): δ 7.52 (2H, dd), 7.08 (2H, t), 4.16 (2H, qua), 3.75 (2H, s), 2.53 (1H, sept), 2.07-2.18 (2H, m), 1.85-2.02 (4H, m), 1.70-1.84 (2H, m), 1.29 (3H, t); UPLC-MS: 0.76 min, 322 [M+H]+, 643 [2M+H]+.

Cis isomer: 1H-NMR (400 MHz, CDCl₃): δ 7.51 (2H, dd), 7.08 (2H, t), 4.16 (2H, qua), 3.72 (2H, s), 2.31-2.43 (1H, m), 2.12-2.21 (2H, m), 1.92-2.11 (4H, m), 1.58-1.69 (2H, m), 1.29 (3H, t); UPLC-MS: 0.75 min, 322 [M+H]+, 643 [2M+H]+.

### Intermediate 44: (Trans)-N-(2-amino-5-cyanophenyl)-2-oxo-3-(2-pyridinyl)-1-oxa-3-azaspiro[4.5]decane-8-carboxamide

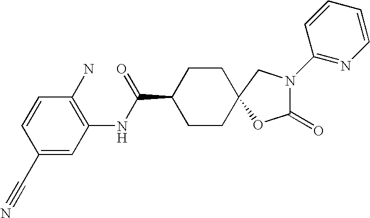

To a shaken mixture of (trans)-2-oxo-3-(2-pyridinyl)-1-oxa-3-azaspiro[4.5]decane-8-carboxylic acid (Intermediate 45, 110 mg, 0.398 mmol) and 3,4-diaminobenzonitrile (commercially available, 80 mg, 0.597 mmol) in anhydrous pyridine (1.6 ml) was added EDC.HCl (153 mg, 0.796 mmol) and the reaction mixture was shaken at room temperature for 1 hour. The mixture was concentrated under vacuum and the residue was partitioned between saturated sodium bicarbonate solution and EtOAc (2×10 ml). The combined organic extracts were washed with brine, dried (Na₂SO₄), filtered and concentrated under vacuum to give a residue which was purified by silica gel chromatography eluting with 40% Et₂O\DCM, Et₂O 100% then 10% MeOH\DCM to afford the title compound as solid (155 mg, 85%). 1H-NMR (400 MHz, DMSO-d6): δ 1.59-1.79 (m, 4H), 1.93-2.02 (m, 2H), 2.03-2.10 (m, 2H), 4.03-4.07 (s, 2H), 5.92 (br s., 2H), 6.77 (d, 1H), 7.13-7.18 (m, 1H), 7.29 (d, 1H), 7.62 (s, 1H), 7.82-7.88 (m, 1H), 8.11 (d, 1H), 8.39 (d, 1H), 9.09-9.16 (s, 1H); UPLC-MS: 0.60 min, m/z 392 [M+H]+.

### Intermediate 45: (Trans)-2-oxo-3-(2-pyridinyl)-1-oxa-3-azaspiro[4.5]decane-8-carboxylic acid

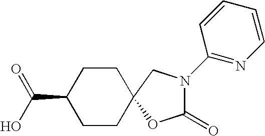

To a stirred solution of ethyl(trans)-2-oxo-3-(2-pyridinyl)-1-oxa-3-azaspiro[4.5]decane-8-carboxylate (Intermediate 46, 232 mg, 0.762 mmol) in methanol (5 ml) was added dropwise a solution of lithium hydroxide (91 mg, 3.81 mmol) in water (1 ml) and the mixture was stirred at room temperature for 45 min. The mixture was concentrated under vacuum, diluted with water and acidified to pH 5 with 1M HCl. The suspension was extracted with EtOAc and the combined organic extracts were washed with brine, dried (Na₂SO₄) and concentrated under vacuum to give the title compound as white powder (214 mg, 98%). 1H-NMR (400 MHz, CDCl₃): δ 1.76-2.22 (m, 8H), 2.53-2.62 (m, 1H), 4.03-4.07 (s, 2H), 7.05-7.10 (m, 1H), 7.75 (m, 1H), 8.30 (d, 1H), 8.36 (d, 1H); UPLC-MS: 0.55 min, m/z 277 [M+H]+.

### Intermediate 46: Ethyl(trans)-2-oxo-3-(2-pyridinyl)-1-oxa-3-azaspiro[4.5]decane-8-carboxylate

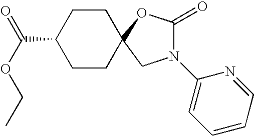

Ethyl(trans)-2-oxo-1-oxa-3-azaspiro[4.5]decane-8-carboxylate (prepared in an analogous method to Intermediate 38, 0.53 g, 2.332 mmol) was dissolved in 8 ml of toluene and 2-bromopyridine (368 mg, 2.332 mmol), copper(I) iodide (22.21 mg, 0.117 mmol), (+/−)-trans-1,2-diaminocyclohexane (26.6 mg, 0.233 mmol) and cesium carbonate (1.52 g, 4.66 mmol) were added and the mixture was heated at 150° C. for 30 min in a microwave oven for 4 cycles. The reaction was poured into water (20 ml) and extracted with ethyl acetate (2×60 ml). The combined organic phases were washed with aq. HCl (1M, 20 ml) then dried (Na₂SO₄) and concentrated under reduced pressure to give 670 mg of crude. The crude was purified on silica gel Flash 25M with SP1 eluting with cyclohexane:EtOAc 7:3. Two compounds were isolated after purification, the trans isomer (Intermediate 46, 325.0 mg, 41%) and the cis isomer (60 mg). 1H-NMR (500 MHz, CDCl₃): δ 1.28 (t, 3H), 1.59-1.62 (m, 2H), 1.73-1.83 (m, 2H), 1.89 (td, 2H), 2.07-2.14 (m, 2H), 2.45-2.52 (m, 1H), 4.03-4.05 (m, 2H), 4.17 (q, 2H), 7.03-7.07 (m, 1H), 7.72 (t, 1H), 8.26 (d, 1H), 8.33 (d, 1H); MS: m/z 305 [M+H]+.

### Intermediate 47: (Trans)-N-{2-amino-4-[(trifluoromethyl)oxy]phenyl}-2-oxo-3-(2-pyridinyl)-1-oxa-3-azaspiro[4.5]decane-8-carboxamide

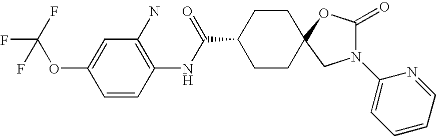

To a stirred mixture of (trans)-2-oxo-3-(2-pyridinyl)-1-oxa-3-azaspiro[4.5]decane-8-carboxylic acid (Intermediate 45, 158 mg, 0.572 mmol) and 4-[(trifluoromethyl)oxy]-1,2-benzenediamine (Intermediate 32, 165 mg, 0.858 mmol) in pyridine (3.6 ml) was added EDC.HCl (175 mg, 0.915 mmol). The mixture was stirred at r.t. for 2 hours. The mixture was concentrated under vacuum then partitioned between aqueous sodium bicarbonate solution and ethyl acetate (×2). The combined organics were washed (water), dried on Na₂SO₄ and concentrated under vacuum. The crude was purified by chromatography (˜20-50% EtOAc\cyclohexane) to give the title compound (163 mg, 63%). 1H-NMR (400 MHz, CDCl₃): δ 1.61-2.23 (m, 11H), 2.43-2.56 (m, 1H), 4.09-4.16 (m, 2H), 6.61-6.71 (m, 1H), 7.01-7.07 (m, 1H), 7.14-7.21 (m, 1H), 7.26-7.35 (m, 1H), 7.69-7.76 (m, 1H), 8.20-8.27 (m, 1H), 8.31-8.39 (m, 1H).

### Intermediate 48: Mixture of (trans)-N-(2-amino-4-cyanophenyl)-2-oxo-3-phenyl-1-oxa-3-azaspiro[4.5]decane-8-carboxamide and (trans)-N-(2-amino-5-cyanophenyl)-2-oxo-3-phenyl-1-oxa-3-azaspiro[4.5]decane-8-carboxamide

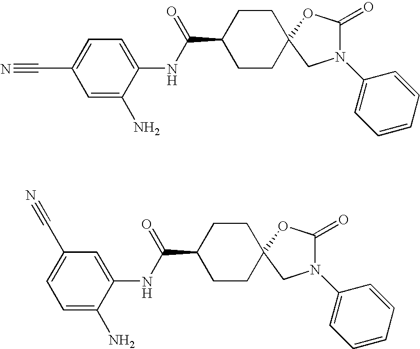

(Trans)-2-oxo-3-phenyl-1-oxa-3-azaspiro[4.5]decane-8-carboxylic acid (Intermediate 10, alternative preparation, 250 mg of mixture assumed to contain 0.300 mmol of desired starting material), 3,4-diaminobenzonitrile (commercially available, 59.9 mg; 0.450 mmol) and EDC (92 mg; 0.479 mmol) were dissolved in pyridine (2 ml) and stirred at room temperature overnight. LC-MS showed starting material was still present in the reaction; 30 mg of 3,4-diaminobenzonitrile and 45 mg of EDC were added and the mixture was stirred at r.t. overnight. The mixture was dried and purified by chromatography: Biotage SP1 on a NH column 25+M eluting with DCM; DCM\Et₂O 9:1 to 7:3 to afford a brown gum (0.202 g) as a complex mixture containing impurities by NMR and LC-MS. It was assumed to contain ˜0.3 mmol of target compounds (as it was assumed for the impure starting material) and the mixture was used for the next step without further purification. UPLC-MS peak 1 0.64 min, m/z 391 [M+H]+ and peak 2 0.65 min, m/z 391 [M+H]+.

### Intermediate 49: Mixture of (trans)-N-(2-amino-5-cyanophenyl)-3-(4-fluorophenyl)-2-oxo-1-oxa-3-azaspiro[4.5]decane-8-carboxamide and (trans)-N-(2-amino-4-cyanophenyl)-3-(4-fluorophenyl)-2-oxo-1-oxa-3-azaspiro[4.5]decane-8-carboxamide

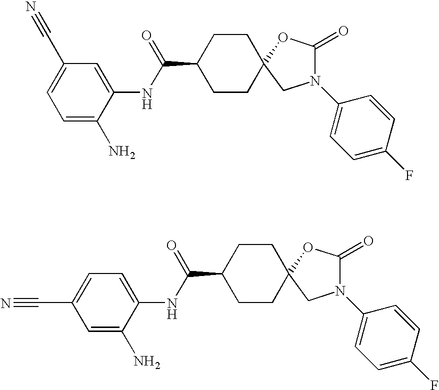

(Trans)3-(4-fluorophenyl)-2-oxo-1-oxa-3-azaspiro[4.5]decane-8-carboxylic acid (prepared in an analogous manner to Intermediate 42, 79 mg, 0.269 mmol) 3,4-diaminobenzonitrile (commercially available, 53.8 mg, 0.404 mmol) and EDC (83 mg, 0.431 mmol) were combined and dissolved in pyridine (2 ml). The brown mixture was stirred at room temperature overnight. The pyridine was removed under reduced pressure then the mixture was partitioned between sat. aq. NaHCO₃ solution and DCM. The DCM extracts (3×1 ml) were combined and dried to give a residue that was purified by silica chromatography (Biotage SP1, 12+M) eluting with cyclohexane\EtOAC 9:1 to 1:1 to afford a brown solid (0.064 g; 58%, undefined mixture of regioisomers by NMR). 1H-NMR (400 MHz, CDCl₃): δ 1.09-1.36 (m, 1H), 1.80-2.02 (m, 4H), 2.06-2.29 (m, 3H), 2.45-2.64 (m, 1H), 3.83 (m, 2H), 4.23-4.35 (m, 1H), 6.79-6.89 (m, 1H), 7.04-7.15 (m, 2H), 7.33-7.41 (m, 1H), 7.45-7.59 (m, 3H); UPLC-MS: 0.66 min, m/z 409 [M+H]+.

### Intermediate 50: (Trans)-N-[2-amino-4-(trifluoromethyl)phenyl]-2-oxo-3-(2-pyridinyl)-1-oxa-3-azaspiro[4.5]decane-8-carboxamide

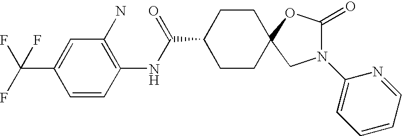

To a stirred mixture of (trans)-2-oxo-3-(2-pyridinyl)-1-oxa-3-azaspiro[4.5]decane-8-carboxylic acid (prepared in an analogous manner to Intermediate 45, 101 mg, 0.366 mmole) and 4-(trifluoromethyl)-1,2-benzenediamine (commercially available, 64.4 mg, 0.366 mmole) in pyridine (3 ml) was added EDC.HCl (112 mg, 0.585 mmole). The mixture was stirred at r.t. overnight. The mixture was concentrated under vacuum then partitioned between aqueous sodium bicarbonate solution (5 ml) and ethyl acetate (2×50 ml). The combined organics were washed (water), dried on Na₂SO₄ and concentrated under vacuum. The crude was purified by chromatography (˜20-50% EtOAc\cyclohexane) to give the title compound (124 mg, 70%). 1H-NMR (400 MHz, DMSO-d6): δ 1.53-1.79 (4H, m), 1.90-2.07 (4H, m), 2.41-2.55 (1H, m), 3.99-4.05 (2H, s), 5.48-5.60 (2H, br s.), 6.76-6.82 (1H, d), 7.09-7.15 (1H, dd), 7.14-7.20 (1H, dd), 7.55-7.57 (1H, d), 7.77-7.84 (1H, m), 8.04-8.10 (1H, d), 8.31-8.38 (1H, dd), 9.09-9.14 (1H, s); MS: m/z 435 [MH]+.

### Intermediate 51: Mixture of (trans)-N-{2-amino-4-[(trifluoromethyl)oxy]phenyl}-3-(6-methyl-2-pyridinyl)-2-oxo-1-oxa-3-azaspiro[4.5]decane-8-carboxamide and (trans)-N-{2-amino-5-[(trifluoromethyl)oxy]phenyl}-3-(6-methyl-2-pyridinyl)-2-oxo-1-oxa-3-azaspiro[4.5]decane-8-carboxamide

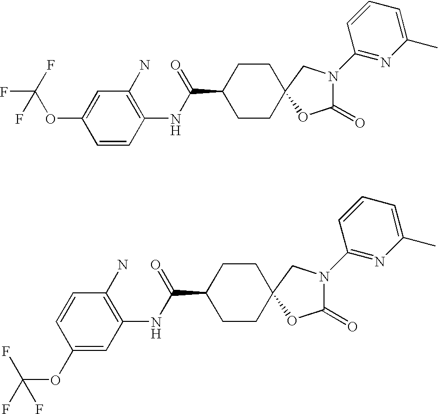

To a shaken mixture of (trans)-3-(6-methyl-2-pyridinyl)-2-oxo-1-oxa-3-azaspiro[4.5]decane-8-carboxylic acid (Intermediate 52, 65.4 mg, 0.225 mmol) and 4-[(trifluoromethyl)oxy]-1,2-benzenediamine (Intermediate 32, 64.9 mg, 0.338 mmol) in pyridine (1.5 ml) was added EDC.HCl (86 mg, 0.451 mmol) and the mixture was shaken for 2 hours. The mixture was concentrated under vacuum; the residue was partitioned between saturated sodium bicarbonate solution and EtOAc. The combined organic extracts were washed with brine, dried (Na₂SO₄) and concentrated under vacuum to give a residue. The residue was purified twice by silica gel chromatography (elution in gradient with 0-100% Et₂O\DCM) to afford the title compound as brown residue (98 mg, 80%). No further purification followed. UPLC-MS: 0.76 min, m/z 465 [M+H]+.

### Intermediate 52: (Trans)-3-(6-methyl-2-pyridinyl)-2-oxo-1-oxa-3-azaspiro[4.5]decane-8-carboxylic acid

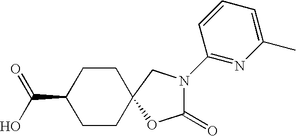

To a stirred solution of ethyl(trans)-3-(6-methyl-2-pyridinyl)-2-oxo-1-oxa-3-azaspiro[4.5]decane-8-carboxylate (Intermediate 53, 70 mg, 0.220 mmol) in MeOH (1.5 ml) a solution of lithium hydroxide (26.3 mg, 1.099 mmol) in water (0.3 ml) was added dropwise and the mixture was stirred at room temperature for 1 hour 10 min. The mixture was concentrated under vacuum, diluted with water (1 ml), acidified to pH 2 with 1M HCl and extracted with EtOAc. The combined organic extracts were washed with brine, dried (Na₂SO₄) and concentrated under vacuum to give the title compound as white solid (65.4 mg, 98%). UPLC-MS: 0.61 min, m/z 291 [M+H]+.

### Intermediate 53: Ethyl(trans)-3-(6-methyl-2-pyridinyl)-2-oxo-1-oxa-3-azaspiro[4.5]decane-8-carboxylate

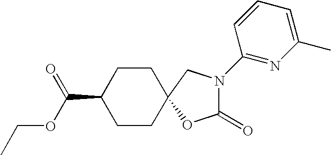

To a solution of ethyl(trans)-2-oxo-1-oxa-3-azaspiro[4.5]decane-8-carboxylate (prepared in an analogous manner to Intermediate 38, 904 mg, 3.98 mmol) and 2-bromo-6-methylpyridine (0.543 ml, 4.77 mmol) in anhydrous 1,4-dioxane (13.5 ml), under a nitrogen atmosphere, CuI (37.9 mg, 0.199 mmol), trans-1,2-diaminocyclohexane (48 μl, 0.398 mmol) and potassium phosphate tribasic (1.69 g, 7.96 mmol) were added and the mixture was stirred at 110° C. overnight. The mixture was diluted with EtOAc, washed with water, dried (Na₂SO₄) and concentrated under vacuum to give a residue which was purified by NH gel chromatography (elution in gradient with 0-100% EtOAc/cyclohexane) to obtain an oil as a mixture of cis-trans isomers. The residue was re-purified by NH gel chromatography eluting in slow gradient with 0-30% EtOAc\cyclohexane then loaded onto a silica column and partially purified with 15% EtOAc\cyclohexane. The mixed fractions were re-purified by silica gel chromatography eluting with 10% EtOAc\cyclohexane. Finally the title compound was obtained as white solid (222 mg, 17.5%). 1H-NMR (400 MHz, CDCl₃): δ 1.29 (t, 3H), 1.74-1.93 (m, 4H), 1.93-2.02 (m, 2H), 2.06-2.16 (m, 2H), 2.45-2.54 (m, 4H), 4.01-4.05 (s, 2H), 4.18 (q, 2H), 6.89 (d, 1H), 7.59 (t, 1H), 8.03 (d, 1H).

### Intermediate 54: (1:3) Mixture (trans)-N-{2-amino-4-[(trifluoromethyl)oxy]phenyl}-3-(2-methyl-3-pyridinyl)-2-oxo-1-oxa-3-azaspiro[4.5]decane-8-carboxamide and (trans)-N-{2-amino-5-[(trifluoromethyl)oxy]phenyl}-3-(2-methyl-3-pyridinyl)-2-oxo-1-oxa-3-azaspiro[4.5]decane-8-carboxamide

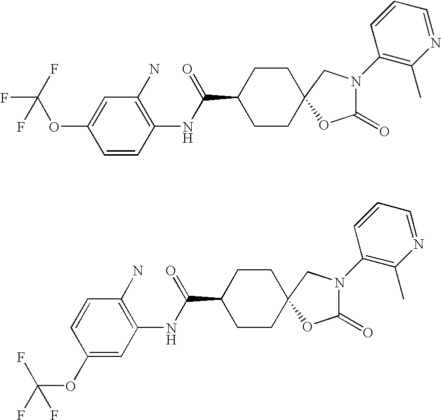

To a stirred mixture of (trans)-3-(2-methyl-3-pyridinyl)-2-oxo-1-oxa-3-azaspiro[4.5]decane-8-carboxylic acid (Intermediate 55, 75 mg, 0.258 mmol) and 4-[(trifluoromethyl)oxy]-1,2-benzenediamine (Intermediate 32, 74.5 mg, 0.388 mmol) in pyridine (3 ml) EDC.HCl (99 mg, 0.517 mmol) was added and the mixture was stirred at room temperature overnight. The mixture was concentrated in vacuo and the residue was partitioned between saturated NaHCO₃ solution and EtOAc (2×). The combined organic extracts were washed with brine, dried over Na₂SO₄, and concentrated under reduced pressure to give a brown residue, which was purified by NH chromatography (eluent EtOAc 100%) affording the title compound as a brown solid (108 mg, 90%). 1H-NMR (400 MHz, DMSO-d6): δ 1.55-1.68 (m, 4H), 1.72-1.83 (m, 4H), 1.92-2.01 (m, 4H), 2.09-2.17 (m, 4H), 2.40-2.45 (s, 6H), 3.85-3.90 (s, 4H), 5.09-5.28 (2×s, together 4H), 6.43-6.91 (m, together 4H), 7.22-7.39 (m, together 4H), 7.83 (d, 2H), 8.40-8.45 (dd, 2H), 9.10-9.17 (2×s, together 2H); UPLC-MS 0.59 min, m/z 465 [M+H]+.

### Intermediate 55: (Trans)-3-(2-methyl-3-pyridinyl)-2-oxo-1-oxa-3-azaspiro[4.5]decane-8-carboxylic acid

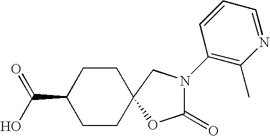

A solution of lithium hydroxide (37.6 mg, 1.571 mmol) in water (0.5 ml) was added dropwise to a solution of ethyl(trans)-3-(2-methyl-3-pyridinyl)-2-oxo-1-oxa-3-azaspiro[4.5]decane-8-carboxylate (Intermediate 56, 100 mg, 0.314 mmol) in MeOH (2 ml) and the mixture was stirred at room temperature for 45 min. The mixture was concentrated in vacuo, diluted with water (2 ml), acidified with 1M HCl (until pH 4.5) and extracted with EtOAc (several times). The combined organic extracts were dried over Na₂SO₄ and concentrated under reduced pressure to give the title compound as a white solid (77 mg, 79%). 1H-NMR (400 MHz, CDCl₃): δ 1.79-1.89 (m, 2H), 1.98-2.05 (m, 4H), 2.13-2.22 (m, 2H), 2.54-2.58 (s, 3H), 2.62-2.69 (m, 1H), 3.69 (s, 2H), 7.21-7.26 (m, 1H), 7.59 (dd, 1H), 8.51 (dd, 1H); UPLC-MS 0.38 min, m/z 291 [M+H]+.

### Intermediate 56: Ethyl(trans)-3-(2-methyl-3-pyridinyl)-2-oxo-1-oxa-3-azaspiro[4.5]decane-8-carboxylate

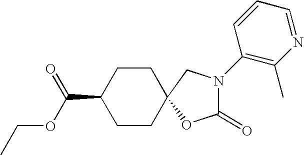

To a stirred solution of 3-bromo-2-methylpyridine (1.01 ml, 8.77 mmol) and ethyl(trans)-2-oxo-1-oxa-3-azaspiro[4.5]decane-8-carboxylate (prepared in an analogous method to Intermediate 38, 1.66 g, 7.30 mmol) in anhydrous 1,4-dioxane (22 ml), CuI (70 mg, 0.365 mmol), trans-cyclohexanediamine (88 μl, 0.730 mmol) and potassium phosphate tribasic (3.10 g, 14.6 mmol) were added and the mixture was heated under a nitrogen atmosphere at 115° C. (external temperature) for 4 hours. Additional CuI (693 mg, 3.65 mmol) and trans-cyclohexanediamine (0.875 ml, 0.730 mmol) were added and the external temperature was increased to 130° C. (internal T=110° C.). Stirring was carried on for further 2 hours. The mixture was diluted with EtOAc and washed with water (2×20 ml), the aq. layer was back-extracted with ethyl acetate and the combined organic extracts were dried over Na₂SO₄, filtered and concentrated under vacuum to obtain a residue. The crude was purified by gradient elution of a NH column (0-100% EtOAc\cyclohexane) to give: 1) a yellowish oil as mixture of trans isomer with traces of cis isomer 2) a colourless oil as a mixture of both isomers and 3) a yellowish solid as cis isomer. The mixed fractions 1) and 2) were combined and separated (twice) by NH chromatography eluting with 0-50% EtOAc\cyclohexane to give the title compound as white solid (478 mg, 20%). 1H-NMR (400 MHz, CDCl₃): δ 1.28 (t, 3H), 1.72-1.82 (m, 2H), 1.93-2.06 (m, 4H), 2.09-2.18 (m, 2H), 2.50-2.59 (m, 4H), 3.69 (s, 2H), 4.16 (q, 2H), 7.20-7.24 (m, 1H), 7.58 (dd, 1H), 8.50 (dd, 1H); UPLC-MS: 0.53 min, m/z 319 [M+H]+.

### Intermediate 57: (Trans)-N-{2-amino-5-[(trifluoromethyl)oxy]phenyl}-2-oxo-3-(2-pyrimidinyl)-1-oxa-3-azaspiro[4.5]decane-8-carboxamide

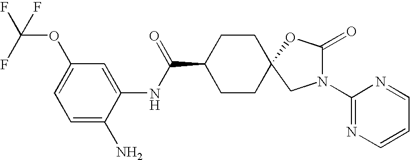

To a stirred solution of (trans)-2-oxo-3-(2-pyrimidinyl)-1-oxa-3-azaspiro[4.5]decane-8-carboxylic acid (Intermediate 58, 0.1247 g, 0.450 mmol) and 4-[(trifluoromethyl)oxy]-1,2-benzenediamine (prepared in an analogous manner to Intermediate 32, 0.130 g, 0.675 mmol) in pyridine (2 ml) was added EDC (0.138 g, 0.720 mmol). The mixture was stirred 2 hours, then left to stand 18 hours. The mixture was partitioned between ethyl acetate (10 ml) and dilute hydrochloric acid (10 ml). The separated aqueous phase was extracted with ethyl acetate (10 ml). The combined organic extracts were washed with water, filtered through a hydrophobic membrane and concentrated under vacuum to give the crude product (0.235 g), which was purified by column chromatography on silica gel, eluting with cyclohexane\ethyl acetate (1:0 to 0:1 gradient followed by 100% ethyl acetate isocratic elution for the product) to give the title compound as a yellow gum (79.1 mg). 1H-NMR (400 MHz, CDCl₃): δ 8.59 (2H, d, J 5 Hz), 8.15 (1H, br s), 7.18 (1H, d, J 9 Hz), 7.04 (1H, t, J 5 Hz), 6.60 (1H, br s), 6.57 (1H, d, J 9 Hz), 4.05 (2H, s), 2.53-2.45 (1H, m), 2.11-2.01 (4H, m), 1.90-1.72 (4H, m); UPLC-MS 0.63 min, m/z 452 [M+H]+.

### Intermediate 58: (Trans)-2-oxo-3-(2-pyrimidinyl)-1-oxa-3-azaspiro[4.5]decane-8-carboxylic acid

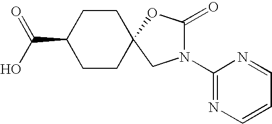

To a stirred solution of ethyl(trans)-2-oxo-3-(2-pyrimidinyl)-1-oxa-3-azaspiro[4.5]decane-8-carboxylate (Intermediate 59, 200 mg, 0.655 mmol) in ethanol (5 ml) was added dropwise sodium hydroxide (1M, 0.622 ml, 0.622 mmol). The mixture was stirred 30 minutes then water (5 ml) was added. The mixture was stirred 2 hours then left to stand overnight. More sodium hydroxide solution was added (0.31 ml) and the mixture was stirred 4 hours then left to stand overnight. The mixture was acidified with 1M HCl to pH 3 and extracted with ethyl acetate (3×10 ml). The organic extracts were filtered through a hydrophobic membrane and concentrated under vacuum to give the title compound, as the major component (60% by 1H-NMR) in a mixture with (trans)-4-hydroxy-4-[(2-pyrimidinylamino)methyl]cyclohexanecarboxylic acid and ethyl(trans)-4-hydroxy-4-[(2-pyrimidinylamino)methyl]cyclohexanecarboxylate. The mixture was used without further purification (0.125 g). 1H-NMR (400 MHz, CDCl₃): δ 8.65 (2H, d, J 5 Hz), 7.04 (1H, t, J 5 Hz), 3.98 (2H, s), 2.55-2.46 (1H, m), 2.14-2.04 (2H, m) and 1.99-1.61 (6H, m).

### Intermediate 59: Ethyl(trans)-2-oxo-3-(2-pyrimidinyl)-1-oxa-3-azaspiro[4.5]decane-8-carboxylate

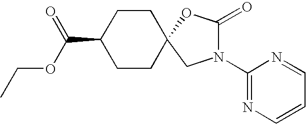

A stirred mixture of ethyl(trans)-2-oxo-1-oxa-3-azaspiro[4.5]decane-8-carboxylate (prepared in an analogous method to Intermediate 38, 1.11 g, 4.88 mmol), 2-bromopyrimidine (0.815 g, 5.13 mmol), copper(I) iodide (0.047 g, 0.244 mmol), trans-1,2-diaminocyclohexane (0.059 ml, 0.488 mmol) and cesium carbonate (3.18 g, 9.77 mmol) in toluene (7 ml) was degassed 3 times (vacuum/nitrogen cycle) then heated to 80° C. and stirred for 24 hours. The mixture was cooled to room temperature, quenched into water (30 ml) and extracted with ethyl acetate (2×30 ml). The combined organic extracts were washed twice with water, filtered through a Phase-Sep filter and concentrated under vacuum to give the crude product (1.478 g), which was purified by column chromatography (silica gel; cyclohexane\ethyl acetate, 1:0 to 10:1 to 3:1 gradient with isocratic steps) to give the title compound (0.683 g). 1H-NMR (400 MHz, CDCl₃): δ 8.68 (2H, d, J 5 Hz), 7.06 (1H, t, J 5 Hz), 4.16 (2H, q, J 7 Hz), 4.01 (2H, s), 2.50 (1H, septet, J 4 Hz), 2.15-2.06 (2H, m), 2.01-1.85 (4H, m), 1.83-1.72 (2H, m) and 1.27 (3H, t, J 7 Hz); UPLC-MS 0.59 min, m/z 306 [M+H]+.

### Intermediate 60: Mixture of (trans)-N-(2-amino-4-bromophenyl)-3-(2-fluorophenyl)-2-oxo-1-oxa-3-azaspiro[4.5]decane-8-carboxamide and (trans)-N-(2-amino-5-bromophenyl)-3-(2-fluorophenyl)-2-oxo-1-oxa-3-azaspiro[4.5]decane-8-carboxamide

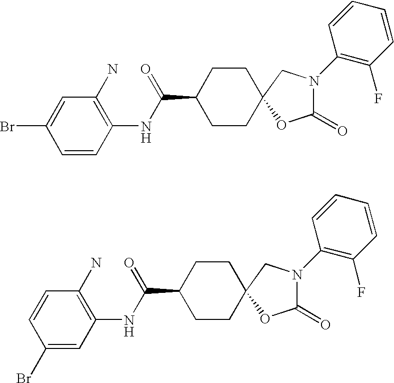

The title compound was made in a similar fashion to the preparation of Intermediate 35 using 4-bromo-1,2-benzenediamine (commercially available from Aldrich, 1.531 g, 8.18 mmol) and stirring the reaction mixture for 20 hours to give the title compound as yellow solid (3.01 g, 36%). 1H-NMR (400 MHz, CDCl₃): δ 1.60 (s, 2H), 1.70-1.89 (m, 2H), 1.90-2.01 (m, 2H), 2.06 (s, 1H), 2.10-2.32 (m, 2H), 2.50 (br s, 2H), 3.85 (s, 2H), 6.81-7.02 (m, 2H), 7.11-7.22 (m, 2H), 7.23-7.34 (m, 2H), 7.54 (t, 2H); UPLC-MS: 0.69 min, m\z 460 and 462 [M+H]+.

### Intermediate 61: 3-[(3,4-dinitrophenyl)oxy]pyridine

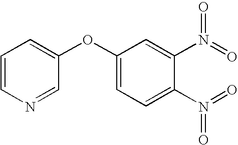

4-fluoro-1,2-dinitrobenzene (1 g, 5.37 mmol), K₂CO₃ (0.891 g, 6.45 mmol) and 3-pyridinol (0.511 g, 5.37 mmol) were placed in a dry flask. Dry DMSO (15 ml) was added and the resulting reaction mixture was stirred at r.t. for 3 hours. The reaction mixture was cooled, washed with saturated ammonium chloride aqueous solution (50 ml) and ethyl acetate (50 ml) was added. The organic phase was separated and dried on Na₂SO₄, filtered and concentrated under vacuum. The crude was purified by silica gel chromatography (Isco-Companion, 40 g silica column) and eluted with ethyl acetate/methanol 1:1 to give the title compound (670 mg, 40.6%). HPLC-MS, 1: 2.031 min, m\z 262.0 [M+H]⁺.

### Intermediate 62: [2-amino-4-(3-pyridinyloxy)phenyl]amine

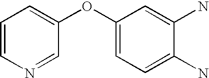

Zinc (0.163 g, 2.489 mmol) in granules and aq 6M HCl (0.415 ml, 2.489 mmol) were added to a solution of 3-[(3,4-dinitrophenyl)oxy]pyridine (Intermediate 61, 0.65 g, 2.489 mmol) in isopropanol (16 ml). The resulting reaction mixture was allowed to react at 40° C. for 14 hours. The reaction mixture was cooled to r.t. and filtered. Ethyl acetate (20 ml) and aq 6M NaOH (1 ml) were added slowly, the mixture was then filtered, the organic phases separated, dried over sodium sulphate and concentrated under vacuum to give a crude. The crude was purified via silica gel chromatography on Isco-Companion system (eluent 100% EtOAc up to EtOAc/MeOH 8:2) to afford the title compound as a yellow oil (220 mg, 39.5%). 1H-NMR (400 MHz, CDCl₃): δ 3.51 (br s, 4H), 6.39-6.51 (m, 2H), 6.71 (d, 1H), 7.19-7.38 (m, 2H), 8.26-8.34 (m, 1H), 8.38 (d, 1H); HPLC-MS, 1: 0.24 min, m\z 202.0 [M+H]⁺.

### Intermediate 63: Sodium (trans)-2-oxo-3-(2-pyridinyl)-1-oxa-3-azaspiro[4.5]decane-8-carboxylate

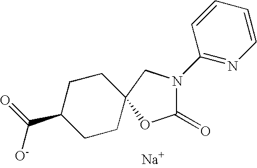

Ethyl(trans)-2-oxo-3-(2-pyridinyl)-1-oxa-3-azaspiro[4.5]decane-8-carboxylate (this can be prepared as described for Intermediate 46, 200 mg, 0.657 mmol) and sodium hydroxide (0.026 g, 0.657 mmol), were placed in a flask containing methanol (10 ml) and water (10 ml). The reaction mixture was stirred at r.t. 24 hours and then it was concentrated under vacuum to give the title compound as a residue which was used in the next step without further purification. HPLC-MS, 1: 1.7 min, m\z 277 [M+H]⁺.

### Intermediate 64: Mixture (trans)-N-[2-amino-5-(3-pyridinyloxy)phenyl]-2-oxo-3-(2-pyridinyl)-1-oxa-3-azaspiro[4.5]decane-8-carboxamide and (trans)-N-[2-amino-4-(3-pyridinyloxy)phenyl]-2-oxo-3-(2-pyridinyl)-1-oxa-3-azaspiro[4.5]decane-8-carboxamide

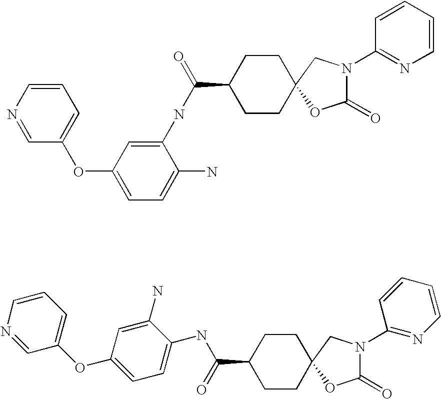

Sodium (trans)-2-oxo-3-(2-pyridinyl)-1-oxa-3-azaspiro[4.5]decane-8-carboxylate (Intermediate 63, 160 mg, 0.535 mmol) was added to pyridine (5 ml) at r.t. [2-amino-4-(3-pyridinyloxy)phenyl]amine (Intermediate 62, 215 mg, 1.069 mmol) was then added and the solution was stirred for 20 min at r.t., then EDC.HCl (205 mg, 1.069 mmol) was added portion wise and the resulting solution was stirred at r.t. for 14 hours. Ethyl acetate (20 ml) and water (20 ml) were added; the organic phase was separated, dried over sodium sulphate, filtered and concentrated under vacuum to give a crude. The crude was purified by silica gel chromatography on Isco-Companion (cyclohexane\ethyl acetate 1:1 to 100% ethyl acetate as eluent) to give the title compound as yellow oil (103 mg, 37.8%). 1H-NMR (400 MHz, CDCl₃): δ 1.86-1.96 (m, 4H), 2.10-2.19 (m, 4H), 2.51 (s, 1H), 3.55 (br s, 2H), 3.93 (s, 2H), 6-40-6.50 (m, 2H), 7.02-7.09 (t, 1H), 7.13-7.15 (d, 1H,) 7.23-7-35 (m, 3H), 7.71-7.76 (t, 1H), 8.25-8.27 (d, 1H), 8.34 (m, 1H), 8.34-8.42 (m, 2H); UPLC-MS: 0.52 min, m\z 460 [M+H]⁺.

### Intermediate 65: Ethyl(trans)-2-oxo-3-(3-pyridinyl)-1-oxa-3-azaspiro[4.5]decane-8-carboxylate

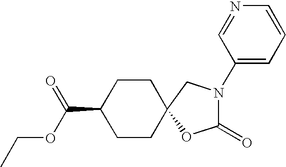

3-bromopyridine (209 mg, 1.320 mmol), ethyl(trans)-2-oxo-1-oxa-3-azaspiro[4.5]decane-8-carboxylate (prepared in a similar fashion to preparation of Intermediate 38, 300 mg, 1.320 mmol), (+\−)-trans-1,2-diaminocyclohexane (15.07 mg, 0.132 mmol) copper(I) iodide (12.57 mg, 0.066 mmol), potassium phosphate tribasic (560 mg, 2.64 mmol) and 1,4-dioxane (20 ml) were collected into a 20 ml microwave vial and stirred at 150° C. for 30 min under microwave irradiation. The reaction was further irradiated at 160° C. for overall 2.5 hours. The reaction mixture was taken up with DCM (100 ml) and washed with water (2×20 ml), then dried and concentrated under vacuum. The resulting crude was purified by Biotage SP1 system, over a 25+M KP-NH cartridge, in gradient with Cy/EtOAc. The title compound was recovered as colourless solid (190 mg, 43%). 1H-NMR (400 MHz, CDCl₃): δ 8.61 (d, 1H), 8.40 (dd, 1H), 8.22 (dq, 1H), 7.33 (ddd, 1H), 4.18 (q, 2H), 3.81 (s, 2H), 2.55 (m, 1H), 2.18-2.09 (m, 2H), 2.04-1.76 (m, 6H), 1.29 (t, 3H); UPLC-MS: 0.56 min, m\z 305 [M+H]+.

The corresponding cis isomer ethyl (cis)-2-oxo-3-(3-pyridinyl)-1-oxa-3-azaspiro[4.5]decane-8-carboxylate was also recovered as colourless solid (120 mg). 1H-NMR (400 MHz, CDCl₃): δ 8.55 (d, 1H), 8.40 (dd, 1H), 8.24 (dq, 1H), 7.33 (ddd, 1H), 4.17 (q, 2H), 3.77 (s, 2H), 2.42-2.33 (m, 1H), 2.21-2.12 (m, 2H), 2.07-1.93 (m, 4H), 1.71-1.61 (m, 2H), 1.29 (t, 3H).

### Intermediate 66: Lithium (trans)-2-oxo-3-(3-pyridinyl)-1-oxa-3-azaspiro[4.5]decane-8-carboxylate

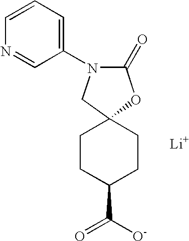

Ethyl(trans)-2-oxo-3-(3-pyridinyl)-1-oxa-3-azaspiro[4.5]decane-8-carboxylate (Intermediate 65, 150 mg, 0.493 mmol) and lithium hydroxide (0.493 ml, 0.493 mmol) were collected in MeOH (2 ml) and stirred at r.t. for 24 hours. The solvent was removed under vacuum to afford 150 mg of crude compound as colourless solid. This was used in the next step without any further purification. 1H-NMR (400 MHz, CDCl₃): δ 8.80 (d, 1H), 8.32 (dd, 1H), 8.05 (dq, 1H), 3.90 (s, 2H), 1.96-1.46 (m, 9H); UPLC-MS: 0.56 min, m\z 275 [M−H]+.

### Intermediate 67: Mixture of (trans)-N-{2-amino-4-[(trifluoromethyl)oxy]phenyl}-2-oxo-3-(3-pyridinyl)-1-oxa-3-azaspiro[4.5]decane-8-carboxamide and (trans)-N-{2-amino-5-[(trifluoromethyl)oxy]phenyl}-2-oxo-3-(3-pyridinyl)-1-oxa-3-azaspiro[4.5]decane-8-carboxamide

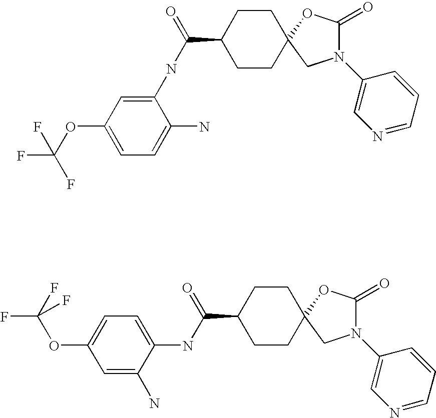

Lithium (trans)-2-oxo-3-(3-pyridinyl)-1-oxa-3-azaspiro[4.5]decane-8-carboxylate (Intermediate 66, 75 mg, 0.251 mmol) and EDC (77 mg, 0.401 mmol) were suspended in pyridine (2 ml) and stirred for 30 min. 4-[(trifluoromethyl)oxy]-1,2-benzenediamine (Intermediate 32, 72.2 mg, 0.376 mmol) was then added and the resulting dark solution was stirred overnight at r.t. Solvent was removed under vacuum, taken up with DCM (20 ml) and washed with saturated NaHCO₃ solution (2×5 ml). The organic phase was concentrated under vacuum to afford a crude oil. The crude was purified by flash chromatography on Biotage SP1 (25+M column, NH cartridge) eluting in gradient with Cyclohexane\EtOAc. The title compound was eluted with 100% EtOAc and recovered as colourless solid (60 mg) as mixture of regioisomers with unknown ratio. 1H-NMR (400 MHz, CD₃OD): δ 8.88 (br s, 2H), 8.34-8.32 (m, 2H), 8.13 (dq, 2H), 7.50-7.45 (m, 2H), 7.18-7.14 (m, 2H), 6.97-6.95 (m, 1H), 6.75-6.73 (m, 1H), 6.58-6.53 (m, 2H), 3.74 (s, 4H), 2.68-1.68 (m, 18H); UPLC-MS: 0.60 min, m\z 226 [M+H]+.

### Intermediate 68: Ethyl(trans)-3-(2-fluoro-3-pyridinyl)-2-oxo-1-oxa-3-azaspiro[4.5]decane-8-carboxylate

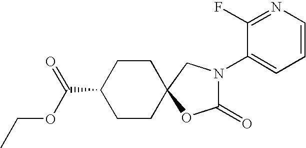

Ethyl 2-oxo-1-oxa-3-azaspiro[4.5]decane-8-carboxylate (prepared in a similar fashion to preparation of Intermediate 40, 1 g, 4.40 mmol, as ˜75/25 mixture of cis\trans isomers), 2-fluoro-3-iodopyridine (0.981 g, 4.40 mmol), copper(I) iodide (0.084 g, 0.440 mmol), (+\−)-trans-1,2-diaminocyclohexane (0.106 ml, 0.880 mmol) and potassium phosphate tribasic (2.80 g, 13.20 mmol) were suspended in 1,4-dioxane (10 ml) and refluxed under N₂ atmosphere at 110° C. The reaction mixture was rinsed with DCM (100 ml) and washed with water, dried over Na₂SO₄, filtered and concentrated. The resulting crude was purified by silica gel chromatography (Biotage SP1, 40+M column), eluting in gradient with Cy/EtOAc. The title compound was eluted with ˜60% EtOAc as single trans stereoisomer (70 mg, 4%). 1H-NMR (400 MHz, CDCl₃): δ 8.15 (dt, 1H), 8.08 (dt, 1H), 7.30 (m, 1H), 4.17 (q, 2H), 3.87 (s, 1H), 2.57-2.49 (m, 1H), 2.17-1.70 (m, 8H), 1.28 (t, 3H); UPLC-MS: 0.66 min, m\z 305 [M+H]+.

It was also recovered a ˜85/15 mixture of ethyl (cis)-3-(2-fluoro-3-pyridinyl)-2-oxo-1-oxa-3-azaspiro[4.5]decane-8-carboxylate and ethyl(trans)-3-(2-fluoro-3-pyridinyl)-2-oxo-1-oxa-3-azaspiro[4.5]decane-8-carboxylate (1.04 g). 1H-NMR (400 MHz, CDCl₃): δ 8.16 (dt, 1H), 8.07 (dt, 1H), 7.27 (ddd, 1H), 4.16 (q, 2H), 3.82 (s, 1H), 2.41-2.31 (m, 1H), 2.24-1.56 (m, 8H), 1.28 (t, 3H); UPLC-MS: 0.65 min, m\z 305 [M+H]+. NMR and UPLC-MS analysis refer to predominant cis isomer (85%).

### Intermediate 69: Lithium (trans)-3-(2-fluoro-3-pyridinyl)-2-oxo-1-oxa-3-azaspiro[4.5]decane-8-carboxylate

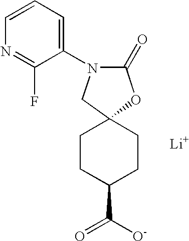

The title compound was made in a similar fashion to preparation of Intermediate 66 using ethyl(trans)-3-(2-fluoro-3-pyridinyl)-2-oxo-1-oxa-3-azaspiro[4.5]decane-8-carboxylate (Intermediate 68, 60 mg, 0.186 mmol) to give the title compound as a colourless solid (60 mg, 100%). 1H-NMR (400 MHz, CDCl₃): δ 8.13 (dt, 1H), 8.06 (dt, 1H), 7.36 (ddd, 1H), 3.90 (s, 2H), 2.26 (m, 1H), 2.14-1.48 (m, 8H); UPLC-MS: 0.50 min, m\z 295 [M+H]+.

### Intermediate 70: Mixture of (trans)-N-{2-amino-5-[(trifluoromethyl)oxy]phenyl}-3-(2-fluoro-3-pyridinyl)-2-oxo-1-oxa-3-azaspiro[4.5]decane-8-carboxamide and (trans)-N-{2-amino-4-[(trifluoromethyl)oxy]phenyl}-3-(2-fluoro-3-pyridinyl)-2-oxo-1-oxa-3-azaspiro[4.5]decane-8-carboxamide

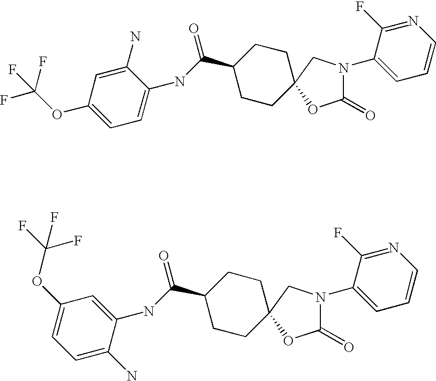

The title compound was made in a similar fashion to preparation of Intermediate 67 using lithium(trans)-3-(2-fluoro-3-pyridinyl)-2-oxo-1-oxa-3-azaspiro[4.5]decane-8-carboxylate (Intermediate 69, 60 mg, 0.204 mmol) to give the title compound as unknown mixture of regioisomers (35 mg, 16%). 1H-NMR (400 MHz, CDCl₃): δ 8.20-8.07 (m, 4H), 7.25-7.13 (m, 4H), 7.71-7.64 (m, 4H), 3.92 (m, 4H), 2.58-2.46 (m, 2H), 2.29-1.71 (m, 16H); UPLC-MS: 0.68 min, 469 m\z [M+H]+.

### Intermediate 71: Ethyl(trans)-3-(2-methylphenyl)-2-oxo-1-oxa-3-azaspiro[4.5]decane-8-carboxylate

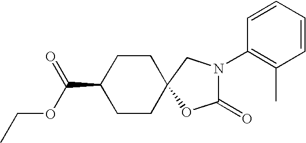

The title compound was made in a similar fashion to preparation of Intermediate 37 using 2-bromotoluene (0.095 ml, 0.792 mmol) to afford the title compound as pale yellow oil (179 mg, 83%). 1H-NMR (400 MHz, CDCl₃): δ 1.28 (t, 3H), 1.68-1.84 (m, 2H), 1.88-2.09 (m, 4H), 2.07-2.21 (m, 2H), 2.32 (br s., 3H), 2.47-2.59 (m, 1H), 3.67 (br s., 2H), 4.16 (q, 2H), 7.17-7.30 (m, 4H); UPLC-MS: 0.75 min, m\z 318 [M+H]⁺.

### Intermediate 72: (Trans)3-(2-methylphenyl)-2-oxo-1-oxa-3-azaspiro[4.5]decane-8-carboxylic acid

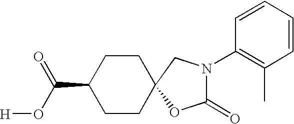

The title compound was made in a similar fashion to preparation of Intermediate 36 using ethyl(trans)-3-(2-methylphenyl)-2-oxo-1-oxa-3-azaspiro[4.5]decane-8-carboxylate (Intermediate 71, 179 mg, 0.564 mmol) to give the title compound as white solid (159 mg, 94%). 1H-NMR (400 MHz, CDCl₃): δ 1.73-1.90 (m, 2H), 1.92-2.06 (m, 4H), 2.10-2.22 (m, 2H), 2.31 (br s, 3H), 2.56-2.68 (m, 1H), 3.66 (br s, 2H), 7.16-7.28 (m, 4H); UPLC-MS: 0.58 min, m\z 290 [M+H]⁺.

### Intermediate 73: (Trans)-N-{2-amino-5-[(trifluoromethyl)oxy]phenyl}-3-(2-methylphenyl)-2-oxo-1-oxa-3-azaspiro[4.5]decane-8-carboxamide

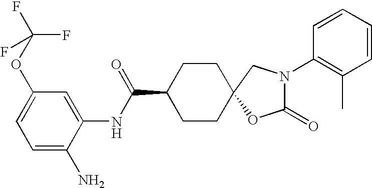

To a mixture of (trans)3-(2-methylphenyl)-2-oxo-1-oxa-3-azaspiro[4.5]decane-8-carboxylic acid (Intermediate 72, 150 mg, 0.518 mmol), {2-amino-4-[(trifluoromethyl)oxy]phenyl}amine (prepared in a similar manner to Intermediate 32, 149 mg, 0.778 mmol) and pyridine (0.126 ml, 1.555 mmol) in anhydrous DCM, EDC. HCl was added and the resulting mixture was stirred at room temperature for 1.5 hour. The mixture was diluted with DCM, washed with saturated NaHCO₃ solution, dried over Na₂SO₄ and concentrated under vacuum to give a residue. The residue was purified by silica gel chromatography eluting in gradient from 20% up to 30% Et₂O\DCM to afford the title compound as yellowish solid (122 mg, 45%). 1H-NMR (400 MHz, DMSO-d6): δ 1.55-1.70 (m, 2H), 1.70-1.82 (m, 2H), 1.90-2.03 (m, 2H), 2.06-2.17 (m, 2H), 2.20-2.27 (s, 3H), 3.83 (br s, 2H), 5.25 (br s, 2H), 6.46 (d, 1H), 6.67 (br s, 1H), 7.21-7.41 (m, 5H), 9.11 (br s, 1H); UPLC-MS: 0.74 min, m\z 464 [M+H]⁺.

### Intermediate 74: Ethyl(trans)-3-(2-chlorophenyl)-2-oxo-1-oxa-3-azaspiro[4.5]decane-8-carboxylate

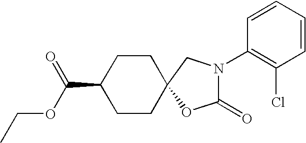

To a solution of ethyl(trans)-2-oxo-1-oxa-3-azaspiro[4.5]decane-8-carboxylate (prepared in a similar manner to Intermediate 38, 100 mg, 0.440 mmol) and 1-bromo-2-chlorobenzene (0.062 ml, 0.528 mmol) in anhydrous 1,4-dioxane (1.5 ml), copper(I) iodide (4.19 mg, 0.022 mmol), (+\−)-trans-1,2-diaminocyclohexane (5.28 μl, 0.044 mmol) and potassium phosphate tribasic (93 mg, 0.44 mmol) were added and the mixture was stirred at 110° C. for 1.5 hour. Additional CuI (tip of spatula) and (+\−)-trans-1,2-diaminocyclohexane (2 drops) and potassium phosphate tribasic (93 mg, 0.44 mmol) were added and the mixture was stirred for further 4 hours and left at room temperature for 60 hours. Additional 1-chloro-2-iodobenzene (54 μl, 0.44 mmol) and (+\−)-trans-1,2-diaminocyclohexane (3 drops) were added and mixture was stirred at 115° C. for further 1.5 hour. The mixture was diluted with EtOAc and washed with water, the aqueous phase was back extracted with EtOAc and the combined organic extracts were dried (Na₂SO₄) and concentrated under vacuum to give a brown residue. The residue was purified by silica gel chromatography eluting with 30% EtOAc\Cyclohexane to afford the title compound as pale yellow oil (111 mg, 70%). 1H-NMR (400 MHz, CDCl3): δ 1.28 (t, 3H), 1.69-1.83 (m, 2H), 1.90-2.19 (m, 6H), 2.46-2.58 (m, 1H), 3.72-3.79 (s, 2H), 4.17 (q, 2H), 7.27-7.39 (m, 2H), 7.41-7.52 (m, 2H); UPLC-MS: 0.74 min, m\z 338 [M+H]⁺.

### Intermediate 75: (Trans)3-(2-chlorophenyl)-2-oxo-1-oxa-3-azaspiro[4.5]decane-8-carboxylic acid

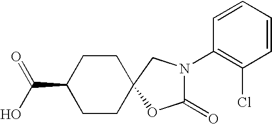

The title compound was made in a similar fashion to preparation of Intermediate 36 using ethyl(trans)-3-(2-chlorophenyl)-2-oxo-1-oxa-3-azaspiro[4.5]decane-8-carboxylate (Intermediate 74, 110 mg, 0.326 mmol) to give the title compound as yellowish oil (103 mg, 100%); UPLC-MS: 0.58 min, m\z 310 [M+H]⁺.

### Intermediate 76: (Trans)N-{2-amino-5-[(trifluoromethyl)oxy]phenyl}-3-(2-chlorophenyl)-2-oxo-1-oxa-3-azaspiro[4.5]decane-8-carboxamide

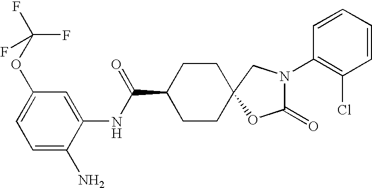

The title compound was made in a similar fashion to preparation of Intermediate 73 using (trans)3-(2-chlorophenyl)-2-oxo-1-oxa-3-azaspiro[4.5]decane-8-carboxylic acid (Intermediate 75, 103 mg, 0.333 mmol) to afford the title compound as yellowish gum (83 mg, 48%). 1H-NMR (400 MHz, DMSO-d6): δ 1.50-1.70 (m, 2H), 1.73-1.86 (m, 2H), 1.90-2.04 (m, 2H), 2.06-2.19 (m, 2H), 3.84 (br s, 2H), 5.26 (br s, 2H), 6.39-6.52 (m, 1H), 6.67 (br s, 1H), 7.25 (d, 1H), 7.36-7.50 (m, 2H), 7.60 (dd, 2H), 9.12 (br s, 1H); UPLC-MS: 0.74 min, m\z 484 [M+H]⁺.

### Intermediate 77: (Trans)-N-(2-amino-4-bromophenyl)-2-oxo-3-(2-pyridinyl)-1-oxa-3-azaspiro[4.5]decane-8-carboxamide

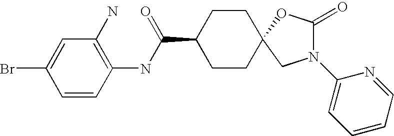

The title compound was made in a similar fashion to preparation of Intermediate 47 using 4-bromo-1,2-benzenediamine (commercially available from Aldrich, 524 mg, 2.80 mmol) to give the title compound (850 mg, 55%). 1H-NMR (400 MHz, CDCl₃): δ 1.33-1.53 (4H, m), 1.60-1.74 (4H, m), 1.95-2.06 (1H, m), 3.39 (1H, br s), 3.63 (2H, s) 4.84-4.87 (1H, m), 6.23-6.27 (1H, m), 6.47 (1H, dd), 6.50-6.54 (1H, m), 6.57-6.65 (1H, m), 6.71 (1H, br s), 7.22-7.30 (1H, m), 7.79 (1H, d), 7.84-7.90 (1H, m); UPLC-MS: 0.64 min, m\z 345 [M+H]⁺

### Intermediate 78: 1,1-Dimethylethyl 5-bromo-2-[(trans)-3-(2-fluorophenyl)-2-oxo-1-oxa-3-azaspiro[4.5]dec-8-yl]-1H-benzimidazole-1-carboxylate

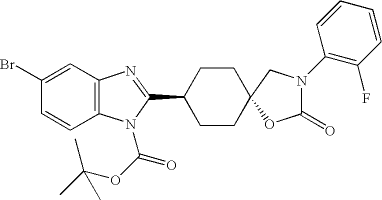

To a solution of (trans)-8-(5-bromo-1H-benzimidazol-2-yl)-3-(2-fluorophenyl)-1-oxa-3-azaspiro[4.5]decan-2-one (Example 55, 100 mg, 0.225 mmol) in DCM (5 ml), TEA (0.031 ml, 0.225 mmol), DMAP (2.75 mg, 0.023 mmol) and BOC-anhydride (0.105 ml, 0.450 mmol) were added and the mixture was stirred at room temperature for 1 hour. Water (5 ml) was added and the organic phase separated and dried over Na₂SO₄. After filtration and evaporation under reduced pressure the title compound was obtained as a white solid and used without any further purification (110 mg, 90%). 1H-NMR (400 MHz, CDCl₃): δ 8.24 (1H, dd), 7.83-8.12 (1H, m), 7.56-7.62 (1H, m), 7.12-7.27 (4H, m), 6.51 (1H, dd), 3.95 (2H, s), 3.62-3.73 (1H, m), 2.18-2.35 (4H, m), 1.98-2.11 (2H, m), 1.81-1.94 (2H, m), 1.75 (9H, d); UPLC-MS: 1.00 min, m\z 544 and 546 [M+H]⁺ and m\z 444 and 446 [M+H-Boc]⁺

### Intermediate 79: Ethyl 3-(6-fluoro-2-pyridinyl)-2-oxo-1-oxa-3-azaspiro[4.5]decane-8-carboxylate

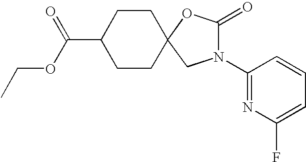

Sodium hydride (17.6 mg, 0.440 mmol) was stirred in dry DMF (1 ml) under nitrogen atmosphere. At 0° C. ethyl(trans)-2-oxo-1-oxa-3-azaspiro[4.5]decane-8-carboxylate (prepared in an analogous manner to Intermediate 38, 100 mg, 0.440 mmol) dissolved in dry DMF (1 ml) was slowly added. A white suspension was formed and the mixture was left stirring while the temperature rose to r.t. (over ˜30 min). Then 2,6-difluoropyridine (0.048 ml, 0.528 mmol) dissolved in dry DMF (0.2 ml) was added and the mixture was stirred at 50° C. for 1.5 hour. One drop of water was added, the solvent was removed under reduced pressure to give a residue that was purified via Biotage SP1 (12+M column) eluting with Cyclohexane\EtOAc 9:1 to 7:3 and affording the title compound as a mixture of trans\cis isomers (1H-NMR shows isomer ratio ˜2:1, relative chemical shifts of CH2N methine groups suggest major isomer is trans isomer) (vitreous solid, 90 mg, 60%). 1H-NMR (400 MHz, CDCl₃): δ 1.21-1.35 (m, 3H), 1.59-2.21 (m, 8H), 2.32-2.44 minor isomer (m, 1H), 2.44-2.56 major isomer (m, 1H), 3.99 major isomer (s, 2H), 3.93 minor isomer (s, 2H), 4.11-4.24 (m, 2H), 6.61-6.71 (m, 1H), 7.74-7.87 (m, 1H), 8.08-8.16 (m, 1H); UPLC-MS: 0.77 min, m/z 323 [M+H]+

### Intermediate 80: Lithium(6-fluoro-2-pyridinyl)-2-oxo-1-oxa-3-azaspiro[4.5]decane-8-carboxylate

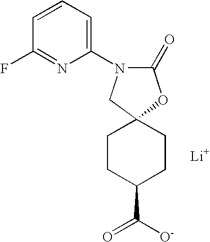

Ethyl 3-(6-fluoro-2-pyridinyl)-2-oxo-1-oxa-3-azaspiro[4.5]decane-8-carboxylate (Intermediate 79, 90 mg, 0.279 mmol) was dissolved in methanol (2 ml). 1.0M aq lithium hydroxide (0.279 ml, 0.279 mmol) was added and the mixture was stirred at r.t. overnight. The mixture was dried under reduced pressure to give the title compound (0.279 mmol, quantitative yield) which was used for the next step without further purification. UPLC-MS: 0.61 min, m/z 295 [M+H]+.

### Intermediate 81: Mixture of N-{2-amino-4-[(trifluoromethyl)oxy]phenyl}-3-(6-fluoro-2-pyridinyl)-2-oxo-1-oxa-3-azaspiro[4.5]decane-8-carboxamide and N-{2-amino-5-[(trifluoromethyl)oxy]phenyl}-3-(6-fluoro-2-pyridinyl)-2-oxo-1-oxa-3-azaspiro[4.5]decane-8-carboxamide

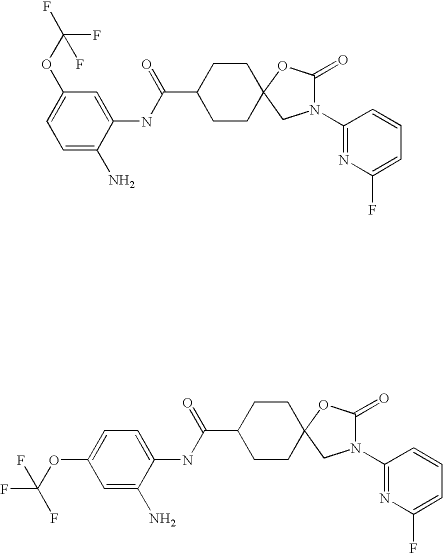

The title compound was made in a similar fashion to preparation of Intermediate 54 using lithium 3-(6-fluoro-2-pyridinyl)-2-oxo-1-oxa-3-azaspiro[4.5]decane-8-carboxylate (Intermediate 80, 0.279 mmol, 84 mg) to give the title compound as a brownish solid (84 mg, assumed mixture of regio and stereoisomers with unknown ratio) which was used in the next step without any further purification. UPLC-MS: peak 1 0.74 min, m/z 469, [M+H]+; peak 2 0.75 min, m/z 469 [M+H]+

### Intermediate 82: Mixture N-(5-amino-2-chloro-4-pyrimidinyl)-2-oxo-3-phenyl-1-oxa-3-azaspiro[4.5]decane-8-carboxamide and N-(4-amino-2-chloro-5-pyrimidinyl)-2-oxo-3-phenyl-1-oxa-3-azaspiro[4.5]decane-8-carboxamide

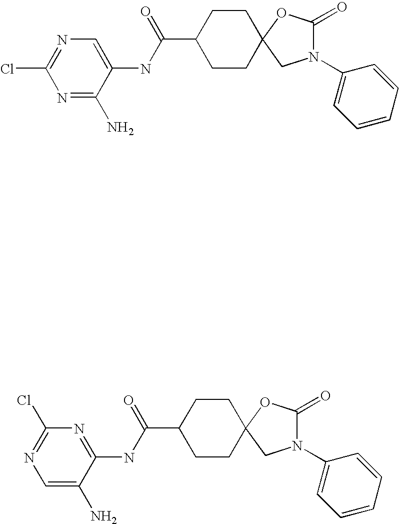

(Trans)-2-oxo-3-phenyl-1-oxa-3-azaspiro[4.5]decane-8-carboxylic acid (Intermediate 10 alternative preparation, 239 mg, 0.286 mmol), 2-chloro-4,5-pyrimidinediamine (commercially available, 41.4 mg, 0.286 mmol) and EDC HCl (88 mg, 0.458 mmol) were dissolved in pyridine (2 ml) and stirred at room temperature overnight. The solvent was removed under vacuum to give a residue which was purified by silica gel chromatography (5 g cartridge) eluting in gradient with Cyclohexane\EtOAc 9:1 to 1:1 then DCM\MeOH 95:5 to afford the title compound as a pale yellow solid (35 mg, 30%), mixture of regio and stereoisomers with unknown ratio). UPLC-MS: 0.60 min, m/z 402 [M+H]+.

### Intermediate 83: Ethyl(trans)-3-(6-fluoro-2-pyridinyl)-2-oxo-1-oxa-3-azaspiro[4.5]decane-8-carboxylate

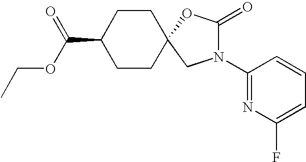

Sodium hydride (0.132 g, 3.30 mmol) was stirred in dry DMF (10 ml) under nitrogen atmosphere. At 0° C. ethyl(trans)-2-oxo-1-oxa-3-azaspiro[4.5]decane-8-carboxylate (prepared in an analogous manner to Intermediate 38, 0.75 g, 3.30 mmol) dissolved in dry DMF (10 ml) was slowly added. A white suspension was formed and the mixture was left stirring while the temperature rose to r.t. (˜30 min). Then 2,6-difluoropyridine (0.359 ml, 3.96 mmol) dissolved in dry DMF (1 ml) was added and the mixture was stirred at 50° C. for 1.5 hour. The mixture was cooled to room temperature. Water (few drops) was added and solvent was removed under reduced pressure to give a residue. The residue was purified via Biotage SP1 (25+M silica column) eluting with cyclohexane\EtOAc 9:1 to 7:3 (several purification runs) to afford the title compound (210 mg, 19%). 1H-NMR (500 MHz, CDCl₃): δ 1.28 (t, 3H), 1.71-1.82 (m, 2H), 1.83-1.93 (m, 2H), 1.90-2.04 (m, 2H), 2.06-2.18 (m, 2H), 2.40-2.56 (m, 1H), 3.98 (s, 2H), 4.15 (q, 2H), 6.66 (dd, 1H) 7.78-7.84 (m, 1H), 8.12 (d, 1H); UPLC-MS (basic conditions) 0.80 min, m/z 323 [M+H]+

### Intermediate 84 3-(6-fluoro-2-pyridinyl)-2-oxo-1-oxa-3-azaspiro[4.5]decane-8-carbaldehyde

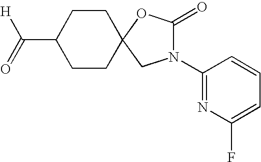

Ethyl(trans)-3-(6-fluoro-2-pyridinyl)-2-oxo-1-oxa-3-azaspiro[4.5]decane-8-carboxylate (Intermediate 83, 210 mg, 0.652 mmol) was dissolved in THF (8 ml) under nitrogen atmosphere and cooled to −78° C. 1M lithium aluminium hydride (0.489 ml, 0.489 mmol) in THF was slowly added and the mixture was stirred at −78° C. for 2.5 hours. Then the mixture was diluted with Et₂O (10 ml) and treated with sodium sulfate decahydrate under vigorous stirring until the temperature rose to r.t. (˜3 h). The precipitate was filtered off by a separator tube, washed with Et₂O and the resulting solution was concentrated under vacuum to give a crude. The crude was purified by silica gel chromatography (Biotage SP1 system, 12+M column) eluting in gradient with 0% to 30% EtOAc\Cyclohexane to afford the title compound as a white solid (29 mg, 16%) as a ˜1:2 mixture of trans and cis isomers, respectively. 1H-NMR (400 MHz, CDCl₃): δ 1.54-1.77 (m, 2H), 1.76-2.04 (m, 4H), 2.04-2.24 (m, 2H), 2.25-2.40 major isomer (m, 1H), 2.40-2.55 minor isomer (m, 1H), 3.93 minor isomer (s, 2H), 3.96 major isomer (s, 2H), 6.60-6.70 (m, 1H), 7.70-7.94 (m, 1H), 8.01-8.18 (m, 1H), 9.67 major isomer (s, 1H), 9.73 minor isomer (s, 1H); UPLC-MS: 0.68 min, m/z 279 [M+H]+.

The alcohol (trans)-3-(6-fluoro-2-pyridinyl)-8-(hydroxymethyl)-1-oxa-3-azaspiro[4.5]decan-2-one (74 mg, 40%) was also recovered as a white solid. UPLC-MS: 0.61 min, m\z 281 [M+H]+.

### Intermediate 85: (Trans)-N-[5-amino-2-(trifluoromethyl)-4-pyridinyl]-2-oxo-3-(2-pyridinyl)-1-oxa-3-azaspiro[4.5]decane-8-carboxamide and (trans)-N-[4-amino-6-(trifluoromethyl)-3-pyridinyl]-2-oxo-3-(2-pyridinyl)-1-oxa-3-azaspiro[4.5]decane-8-carboxamide

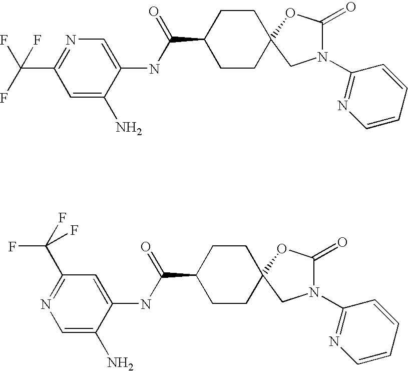

(Trans)-2-oxo-3-(2-pyridinyl)-1-oxa-3-azaspiro[4.5]decane-8-carboxylic acid (prepared in a similar manner to Intermediate 45, 60 mg, 0.217 mmol) and 6-(trifluoromethyl)-3,4-pyridinediamine (this can be prepared as described in WO2006/065703, 57.7 mg, 0.326 mmol) were dissolved in pyridine (1.6 ml). EDC HCl (66.6 mg, 0.347 mmol) was added and the mixture was shaken at room temperature overnight. The mixture was concentrated under vacuum, treated with DCM (2 ml) and saturated NaHCO₃ aqueous solution. After extractions with DCM (3×2 ml) the organic extracts were combined and dried to afford the title compounds as a mixture which was submitted to next step without any further purification (32 mg, 34%). UPLC-MS: 0.60 min, m/z 436, [M+H]+

### Intermediate 86: N-[2-amino-5-(trifluoromethyl)-3-pyridinyl]-2-oxo-3-(2-pyridinyl)-1-oxa-3-azaspiro[4.5]decane-8-carboxamide and N-[3-amino-5-(trifluoromethyl)-2-pyridinyl]-2-oxo-3-(2-pyridinyl)-1-oxa-3-azaspiro[4.5]decane-8-carboxamide

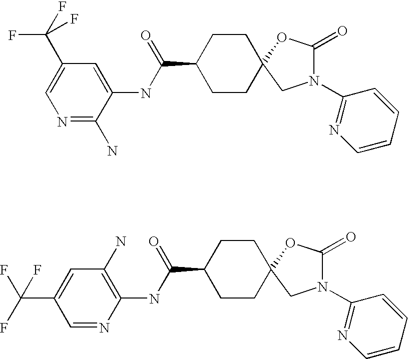

The title compound was made in a similar fashion to preparation of Intermediate 85 using 5-(trifluoromethyl)-2,3-pyridinediamine hydrochloride (this can be prepared as described in J. Med. Chem. 1997, 40(22), 3684, 46.4 mg, 0.217 mmol) to give the title compound as a mixture of regio and stereoisomers with unknown ratio (yellow foam, 55 mg; 56%). 1H-NMR (400 MHz, CDCl₃): δ 1.78-2.03 (m, 4H), 2.05-2.25 (m, 4H), 2.45-2.59 (m, 1H), 4.10 (s, 2H), 4.88-5.11 (m, 2H), 7.01-7.10 (m, 1H), 7.12-7.21 (m, 1H), 7.66-7.77 (m, 2H), 8.19-8.25 (m, 1H), 8.25-8.30 (m, 1H), 8.30-8.38 (m, 1H); UPLC-MS: 0.66 min, m/z 436 [M+H]+.

### EXAMPLES

### Example 1: 8-(1H-Benzimidazol-2-yl)-3-phenyl-1-oxa-3,8-diazaspiro[4.5]decan-2-one

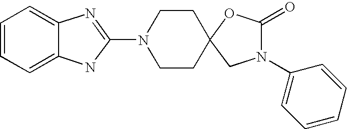

Commercially available 2-chloro-1H-benzimidazole (16.3 mg, 0.107 mmol) was dissolved in DMSO (0.7 ml). 3-Phenyl-1-oxa-3,8-diazaspiro[4.5]decan-2-one hydrobromide (for a preparation see U.S. Pat. No. 4,244,961, 40.1 mg, 0.128 mmol) and DIPEA (0.056 ml) were added. The mixture was irradiated in a microwave at 120° C. for 10 min. followed by three cycles at 150° C. irradiating first for 20 min, and then twice for 30 min. The mixture was diluted with EtOAc (20 ml) and washed with ice-water (3×5 ml), sat. aq. NaHCO₃ (5 ml) and brine (5 ml). The organic phase was dried (Na₂SO₄), filtered and evaporated. The residue was purified by silica gel chromatography eluting with cyclohexane\EtOAc\MeOH: 1\1\0 to 9\9\1 to give the title compound (14.0 mg, 37.6%). 1H-NMR (500 MHz, DMSO-d6): δ 11.41 (1H, br s), 7.56 (2H, d), 7.39 (2H, t), 7.21 (1H, d), 7.17 (1H, d), 7.12 (1H, t), 6.94 (1H, t), 6.89 (1H, t), 3.92 (2H, s), 3.73-3.81 (2H, m), 3.50-3.60 (2H, m), 1.90-2.02 (4H, m); HPLC-MS, 2: 2.28 min, m/z 439 [M+H]+, m\z 437 [M−H]−.

### Example 2

### 3-Phenyl-8-[5-(trifluoromethyl)-1H-benzimidazol-2-yl]-1-oxa-3,8-diazaspiro-[4.5]decan-2-one

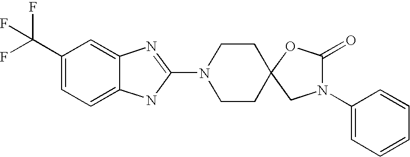

2-Chloro-5-(trifluoromethyl)-1H-benzimidazole (Intermediate 1, 50 mg, 0.227 mmol), which is commercially available or can be prepared according to the procedure described herein, was dissolved in DMSO (1.5 ml). 3-Phenyl-1-oxa-3,8-diazaspiro[4.5]decan-2-one hydrobromide (for a preparation see U.S. Pat. No. 4,244,961, 78.1 mg, 0.249 mmol) and DIPEA (0.119 ml) were added. The mixture was irradiated in a microwave twice at 150° C. for 30 min. The mixture was passed through a SPE-SCX cartridge eluting with DCM, MeOH and 2M ammonia in methanol in this order. The basic fractions were evaporated and the residue was purified by silica gel chromatography eluting with cyclohexane/EtOAc/MeOH: 1/1/0 to 9/9/2 to give the title compound (20.0 mg, 21.2%). 1H-NMR (500 MHz, DMSO-d6): δ 11.86 (1H, br s), 7.57 (2H, d), 7.45 (1H, br s), 7.40 (2H, t), 7.34 (1H, d), 7.26 (1H, d), 7.14 (1H, t), 3.93 (2H, s), 3.81-3.88 (2H, m), 3.55-3.65 (2H, m), 1.91-2.06 (4H, m); HPLC-MS, 2: 2.69 min, m/z 417 [M+H]+.

### Example 3

### (Cis)-3-phenyl-8-[5-(trifluoromethyl)-1H-benzimidazol-2-yl]-1-oxa-3-azaspiro[4.5]decan-2-one

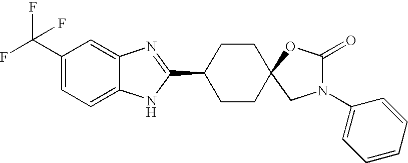

A mixture of (cis)-N,N′-[4-(trifluoromethyl)benzene-1,2-diyl]bis(2-oxo-3-phenyl-1-oxa-3-azaspiro[4.5]decane-8-carboxamide) (Intermediate 3, 25 mg, 36 μmol) and polyphosphoric acid (1 ml) was heated to 110° C. and shaken for 4 hours. The mixture was cooled to r.t. and partitioned between aqueous sodium hydroxide solution and ethyl acetate (2 portions). The combined organic extracts were washed (brine) and concentrated under vacuum to give a residue (10.2 mg) which was purified by MDAP to give the title compound (2.7 mg, 18%). 1H-NMR (400 MHz, CD₃OD): δ 8.56 (1H, m, NH), 7.85 (1H, br s), 7.68 (1H, br d, J 8 Hz), 7.60 (2H, d, J 8 Hz), 7.51 (1H, d, J 8 Hz), 7.40 (2H, t, J 8 Hz), 7.17 (1H, t, J 8 Hz), 3.92 (2H, s), 3.14 (1H, m), 2.29-2.13 (6H, m) and 2.00-1.91 (2H, m); UPLC-MS: 0.63 min, m/z 416 [M+H]+.

### Example 4

### (Trans)-3-phenyl-8-[5-(trifluoromethyl)-1H-benzimidazol-2-yl]-1-oxa-3-azaspiro[4.5]decan-2-one hydrochloride

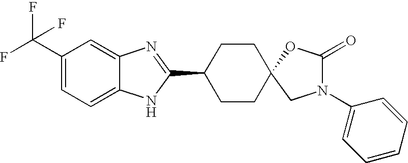

To a solution of (trans)-3-phenyl-8-[5-(trifluoromethyl)-1H-benzimidazol-2-yl]-1-oxa-3-azaspiro[4.5]decan-2-one (Example 5, 70 mg, 0.169 mmol) in methanol (2 ml) was added a solution of hydrogen chloride in ether (1M, 0.2 ml, 0.2 mmol). The solution was left to stand 10 minutes then concentrated under a flow of nitrogen and dried for 18 hours under high vacuum at 40° C. to give the title compound as a white solid (70.1 mg). 1H-NMR (400 MHz, CD₃OD): δ 8.13 (1H, s), 7.99 (1H, d, J 8 Hz), 7.91 (1H, d, J 8 Hz), 7.62 (2H, d, J 8 Hz), 7.41 (2H, t, J 8 Hz), 7.18 (1H, t, J 8 Hz), 4.06 (2H, s), 3.48 (1H, m), 2.47-2.39 (2H, m), 2.26-2.20 (2H, m), 2.14-2.02 (4H, m); UPLC-MS: 0.67 min, m/z 416 [M+H]+.

### Example 5

### (Trans)-3-phenyl-8-[5-(trifluoromethyl)-1H-benzimidazol-2-yl]-1-oxa-3-azaspiro[4.5]decan-2-one

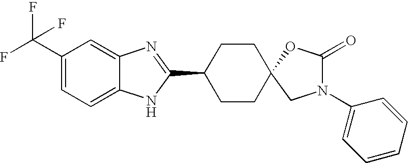

A solution of (trans)-N-[2-amino-5-(trifluoromethyl)phenyl]-2-oxo-3-phenyl-1-oxa-3-azaspiro[4.5]decane-8-carboxamide (Intermediate 9, 0.125 g, 0.144 mmol) and p-toluenesulfonic acid (2.74 mg, 0.014 mmol) in 1,4-dioxane (1 ml) and toluene (1 ml) was heated to 150° C. in a microwave and stirred for 30 minutes. The crude product was added to an SCX-2 ion exchange column (1 g) and was eluted with 1) methanol (20 ml) followed by 2) 2M ammonia/methanol (20 ml). The collected basic fractions were concentrated under vacuum to give the crude product (111 mg). The crude was added to a silica gel column which was eluted with dichloromethane\ethyl acetate (0-10%) to give the title compound (74.3 mg, 62%). 1H-NMR (400 MHz, CDCl₃): δ 7.87 (1H, br s), 7.64 (1H, d, J 8 Hz), 7.53 (2H, d, J 8 Hz), 7.50 (1H, d, J 8 Hz), 7.39 (2H, t, J 8 Hz), 7.17 (1H, t, J 8 Hz), 3.87 (2H, s), 3.08 (1H, m), 2.35-2.25 (2H, m), 2.19-1.91 (6H, m); UPLC-MS: 0.68 min, m/z 416 [M+H]+.

### Example 6

### 8-(5-bromo-1H-benzimidazol-2-yl)-3-phenyl-1-oxa-3,8-diazaspiro[4.5]decan-2-one

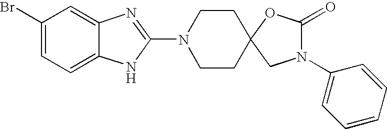

A mixture of 3-phenyl-1-oxa-3,8-diazaspiro[4.5]decan-2-one hydrobromide (for a preparation see U.S. Pat. No. 4,244,961, 100 mg, 0.32 mmol), 5-bromo-2-chloro-1H-benzimidazole (available on the market, 70 mg), diisopropylethylamine (0.17 ml) and dimethylsulfoxide (1 ml) was heated to 180° C. in a microwave reactor and stirred for 40 minutes. The mixture was cooled to room temperature and partitioned between water and ethyl acetate. The organic layer was washed with water, filtered through a hydrophobic membrane (Phase Sep) and concentrated under vacuum to give the crude product (156 mg). The crude was purified by chromatography on silica gel eluting with cyclohexane\ethyl acetate (1:0 to 1:4 gradient). The product-containing fractions were concentrated under vacuum to give a solid (48 mg), which was triturated with 1:1 cyclohexane-DCM, then dried under vacuum at 40° C. for 2 hours to give the title compound (33 mg, 25%). 1H-NMR (400 MHz, DMSO-d6): δ 11.60 (1H, br s), 7.57 (2H, m), 7.40 (2H, m), 7.36 (0.5H, d, J 2 Hz), 7.29 (0.5H, d, J 2 Hz), 7.17-7.07 (3H, m), 7.02 (0.5H, dd, J 2.8 Hz), 3.93 (2H, s), 3.84-3.76 (2H, m), 3.60-3.51 (2H, m), 2.01-1.94 (4H, m); HPLC-MS, 1: 1.614 min, m/z 428 [M+H]+.

### Example 7

### 8-(5,6-Dichloro-1H-benzimidazol-2-yl)-3-phenyl-1-oxa-3,8-diazaspiro-[4,5]decan-2-one

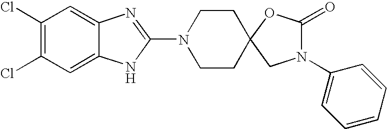

A mixture of 3-phenyl-1-oxa-3,8-diazaspiro[4.5]decan-2-one hydrobromide (for a preparation see U.S. Pat. No. 4,244,961, 50 mg, 0.16 mmol), 2,5,6-trichloro-1H-benzimidazole (Intermediate 12, 34 mg), diisopropylethylamine (84 μl) and dimethylsulfoxide (1 ml) was heated to 180° C. in a microwave reactor and stirred for 40 minutes. The mixture was cooled to room temperature and partitioned between water and ethyl acetate. The organic layer was washed with water, filtered through a hydrophobic membrane (Phase Sep) and concentrated under vacuum to give the crude product (75 mg). The crude was purified by chromatography on silica gel eluting with cyclohexane\ethyl acetate (1:0 to 1:4 gradient). The product-containing fractions were concentrated under vacuum to give a solid (19 mg), which was triturated with 1:1 cyclohexane-DCM, then dried under vacuum at 40° C. for 2 hours to give the title compound (11 mg, 17%). 1H-NMR (400 MHz, DMSO-d6): δ 11.74 (1H, br s), 7.57 (2H, d, J 8 Hz), 7.40 (2H, t, J 8 Hz), 7.40 (1H, br s), 7.32 (1H, br s), 7.13 (1H, t J 8 Hz), 3.93 (2H, s), 3.85-3.78 (2H, m), 3.61-3.52 (2H, m) 2.02-1.92 (4H, m); HPLC-MS, 1: 1.832 min, m/z 417 [M+H]+.

### Example 8

### 8-(5-Fluoro-1H-benzimidazol-2-yl)-3-phenyl-1-oxa-3,8-diazaspiro[4.5]decan-2-one

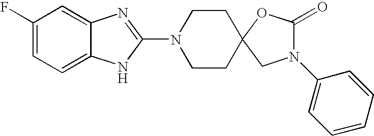

A mixture of 3-phenyl-1-oxa-3,8-diazaspiro[4.5]decan-2-one hydrobromide (for a preparation see U.S. Pat. No. 4,244,961, 50 mg, 0.16 mmol), 2-chloro-5-fluoro-1H-benzimidazole (Intermediate 14, 26 mg), diisopropylethylamine (84 μl) and dimethylsulfoxide (1 ml) was heated to 180° C. in a microwave reactor and stirred for 40 minutes. The mixture was cooled to room temperature and partitioned between water and ethyl acetate. The organic layer was washed with water, filtered through a hydrophobic membrane (Phase Sep) and concentrated under vacuum to give the crude product (72 mg). The crude was purified by chromatography on silica gel eluting with cyclohexane\ethyl acetate (1:0 to 1:4 gradient). The product-containing fractions were concentrated under vacuum to give a solid (17 mg), which was triturated with 1:1 cyclohexane-DCM, then dried under vacuum at 40 C for 2 hours to give the title compound (14 mg, 24%). 1H-NMR (400 MHz, DMSO-d6): δ 11.59 (0.4H, br s), 11.48 (0.6H, br s), 7.57 (2H, d, J 8 Hz), 7.40 (2H, t, J 8 Hz), 7.20-7.09 (2H, m), 7.02-6.96 (1H, m), 6.81-6.67 (1H, m), 3.93 (2H, m), 3.83-3.73 (2H, m), 3.60-3.50 (2H, m), 2.02-1.91 (4H, m); HPLC-MS, 1: 1.472 min, m/z 367 [M+H]+.

### Example 9

### 3-(3,4-Dichlorophenyl)-8-[5-(trifluoromethyl)-1H-benzimidazol-2-yl]-1-oxa-3,8-diazaspiro[4.5]decan-2-one

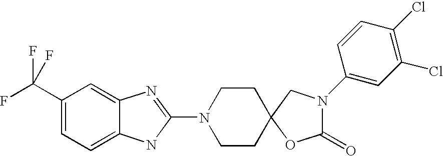

2-Chloro-5-(trifluoromethyl)-1H-benzimidazole (Intermediate 1, 100 mg, 0.45 mmole) and 3-(3,4-dichlorophenyl)-1-oxa-3,8-diazaspiro[4.5]decan-2-one hydrobromide (Intermediate 16, 190.5 mg, 0.498 mmol) were dissolved in a mixture of 2-pentanol:CH₃CN (4:1, 2 ml) in a vial for microwave reactions, DIPEA was added (235.1 μl, 1.35 mmol) and the mixture was heated at 200° C. for 10 minutes. The mixture was concentrated under vacuum; the crude was purified by MDAP, then the fractions were collected and purified by ion-exchange SCX to give the title compound (35.4 mg, 16%). 1H-NMR (500 MHz, CDCl₃): δ 1.89-2.04 (4H, m), 3.57-3.66 (2H, m), 3.76-3.87 (2H, m), 3.95 (2H, s), 7.20-7.51 (3H, m), 7.54-7.59 (1H, m), 7.63-7.69 (1H, d), 7.84-7.86 (1H, d), 11.79 (1H, br s); MS: m/z 485 [M+H]⁺.

### Example 10

### 8-[5-(4-Fluorophenyl)-1H-benzimidazol-2-yl]-3-phenyl-1-oxa-3,8-diazaspiro-[4.5]decan-2-one

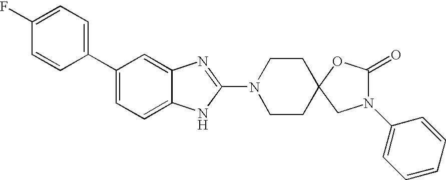

A mixture of 8-(5-bromo-1H-benzimidazol-2-yl)-3-phenyl-1-oxa-3,8-diazaspiro[4.5]decan-2-one (Example 6, 23 mg, 54 μmol), 4-fluorophenylboronic acid (9 mg), tetrakis(triphenylphosphine)palladium (3 mg, 5 mol %), sodium carbonate (17 mg), dioxane (1 ml) and water (0.2 ml) was heated to 140° C. in a microwave reactor and stirred for 10 minutes. A solution of further 4-fluorophenylboronic acid (10 mg) and tetrakis(triphenylphosphine)palladium (2 mg) in dioxane (0.5 ml) was added via syringe and the resultant mixture was heated to 140° C. in a microwave reactor and stirred 30 minutes. The mixture was cooled to r.t., filtered and concentrated under vacuum. The residue was purified by MDAP and then by chromatography on silica gel, eluting with cyclohexane\ethyl acetate (1:0 to 1:4) to give the title compound (3.6 mg, 15%). 1H-NMR (400 MHz, CD₃OD): δ 7.65-7.58 (4H, m), 7.47 (1H, br s), 7.41 (2H, t, J 8 Hz), 7.34 (1H, d, J 8 Hz), 7.29 (1H, d, J 8 Hz), 7.21-7.12 (3H, m), 4.00-3.91 (4H, m), 3.69-3.60 (2H, m) 2.20-2.03 (4H, m); HPLC-MS, 1: 1.825 min, m/z 443 [M+H]+.

### Example 11

### 8-(5-chloro-1H-benzimidazol-2-yl)-3-phenyl-1-oxa-3,8-diazaspiro[4.5]decan-2-one trifluoroacetate

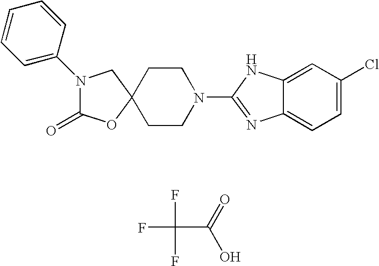

3-Phenyl-1-oxa-3,8-diazaspiro[4.5]decan-2-one hydrobromide (for a preparation see U.S. Pat. No. 4,244,961, 47 mg, 0.15 mmol) and 2,5-dichloro-1H-benzimidazole (commercially available, 19 mg, 0.1 mmol) were dissolved in 2-pentanol (1 ml) and acetonitrile (0.2 ml). The resulting mixture was heated in a microwave at 200° C. for 10 mins. The crude product was purified by mass-directed HPLC; LCMS: 2.41 min, m/z (ES) 383.3 [M+H]⁺.

The following compounds were prepared by a similar procedure to that described for Example 11, from the appropriate secondary amine and the appropriate 2-chlorobenzimidazole.

| | | | Reten- |
|---|---|---|---|
| | | | tion |
| | | | time |
| | Structure | m/z | (min) |
| Ex. | Name | [M + H]⁺ | LC/MS |
| 12 | 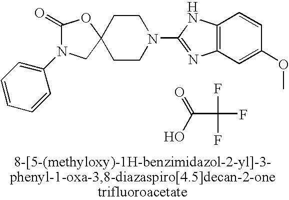 | 379.3 | 2.36 |
| 13 | 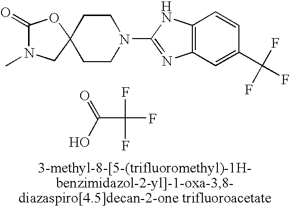 | 355.3 | 2.26 |
| 14 | 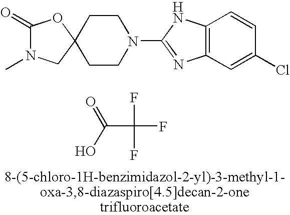 | 321.2 | 2.11 |
| 15 | 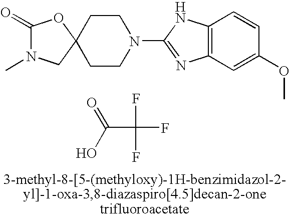 | 317.3 | 2.05 |

### Example 16

### 3-Phenyl-8-{5-[(trifluoromethyl)oxy]-1H-benzimidazol-2-yl}-1-oxa-3,8-diazaspiro[4.5]decan-2-one hydrochloride

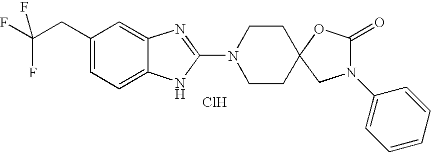

A mixture of 3-phenyl-1-oxa-3,8-diazaspiro[4.5]decan-2-one hydrobromide (for a preparation see U.S. Pat. No. 4,244,961, 100 mg, 0.32 mmol), 2-chloro-5-trifluoromethoxy-1H-benzimidazole (Intermediate 23, 72 mg), diisopropylethylamine (0.17 ml) and dimethylsulfoxide (1 ml) was heated to 180° C. in a microwave reactor and stirred for 40 minutes. The mixture was cooled to room temperature and partitioned between water and ethyl acetate. The organic layer was washed with water, filtered through a hydrophobic membrane (Phase Sep) and concentrated under vacuum to give the crude product (132 mg), which was purified via chromatography on silica gel eluting with dichloromethane\ethyl acetate (1:0 to 10:1 gradient) to give the free base (39.5 mg, 30%). The free base was treated with ethereal hydrogen chloride solution (1M, 2 eq.), left to stand 30 minutes then concentrated under nitrogen flux and dried under vacuum to give the title compound (40.7 mg). 1H-NMR (400 MHz, CD₃OD): δ 7.61 (1H, d, J 8 Hz), 7.49 (1H, d, J 8 Hz), 7.42 (2H, t, J 8 Hz), 7.37 (1H, br s), 7.29 (1H, d, J 8 Hz), 7.19 (1H, t, J 8 Hz), 4.04-3.96 (4H, m), 3.87-3.77 (2H, m), 2.33-2.26 (2H, m), 2.25-2.16 (2H, m); UPLC-MS: 0.59 min, m/z 433 [M+H]+.

### Example 17

### (Trans)-2-phenyl-8-[5-(trifluoromethyl)-1H-benzimidazol-2-yl]-2-azaspiro-[4.5]decan-3-one hydrochloride

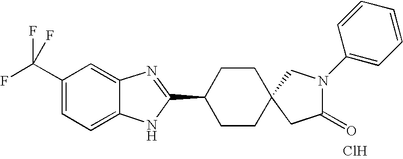

To a solution of (trans)-2-phenyl-8-[5-(trifluoromethyl)-1H-benzimidazol-2-yl]-2-azaspiro[4.5]decan-3-one (Example 19, 47.2 mg) in methanol (1 ml) was added ethereal hydrogen chloride solution (1M, 2 eq.). The mixture was left to stand 30 minutes then concentrated under nitrogen flux and dried under vacuum to give the title compound (50.2 mg) as a white solid. 1H-NMR (400 MHz, CD₃OD): δ 8.12 (1H, s), 7.99 (1H, d, J 8.5 Hz), 7.91 (1H, d, J 8.5 Hz), 7.61 (2H, d, J 8 Hz), 7.40 (2H, t, J 8 Hz), 7.21 (1H, t, J 8 Hz), 3.78 (2H, s), 3.36 (1H, m), 2.69 (2H, s), 2.32-2.24 (2H, m), 2.10-1.91 (4H, m) and 1.86-1.76 (2H, m); UPLC-MS: 0.63 min, m/z 414 [M+H]+.

### Example 18

### (Cis)-2-phenyl-8-[5-(trifluoromethyl)-1H-benzimidazol-2-yl]-2-azaspiro[4.5]-decan-3-one hydrochloride

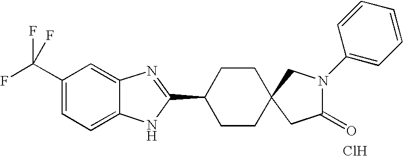

To a solution of (cis)-2-phenyl-8-[5-(trifluoromethyl)-1H-benzimidazol-2-yl]-2-azaspiro[4.5]decan-3-one (Example 20, 18.9 mg) in methanol (1 ml) was added ethereal hydrogen chloride solution (1M, 2 eq.). The mixture was left to stand 30 minutes then concentrated under nitrogen flux and dried under vacuum to give the title compound (20.7 mg) as a white solid, which crystallised slowly. 1H-NMR (400 MHz, CD₃OD): δ 8.11 (1H, s), 7.98 (1H, d, J 8.5 Hz), 7.91 (1H, d, J 8.5 Hz), 7.63 (2H, d, J 8 Hz), 7.42 (2H, t, J 8 Hz), 7.22 (1H, t, J 8 Hz), 3.93 (2H, s), 2.57 (2H, s), 2.31-2.22 (2H, m), 2.13-1.94 (4H, m) 1.84-1.74 (2H, m); UPLC-MS: 0.64 min, m/z 414 [M+H]+.

### Examples 19 and 20

### (Trans)-2-phenyl-8-[5-(trifluoromethyl)-1H-benzimidazol-2-yl]-2-azaspiro[4.5]decan-3-one (Example 19) and (cis) 2-phenyl-8-[5-(trifluoromethyl)-1H-benzimidazol-2-yl]-2-azaspiro[4.5]decan-3-one (Example 20)

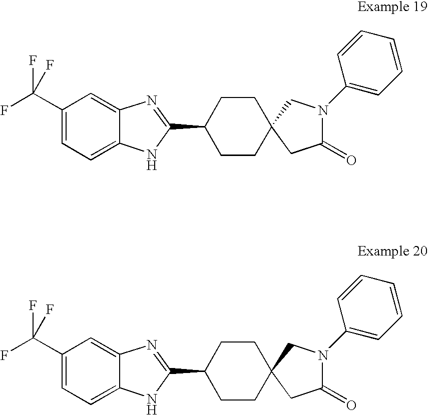

A mixture of N-[2-amino-5-(trifluoromethyl)phenyl]-3-oxo-2-phenyl-2-azaspiro[4.5]decane-8-carboxamide (Intermediate 25, 0.13 g, 0.3 mmol), p-toluenesulfonic acid (10 mg), toluene (0.5 ml) and dioxane (0.5 ml) was heated to 150° C. in a microwave reactor and stirred 40 minutes. The mixture was cooled to r.t. and loaded onto an acidic ion-exchange column (SCX-2), which was eluted with methanol, then with methanolic ammonia solution (2M). The basic washings were concentrated under vacuum, then purified by chromatography on silica gel, eluting with dichloromethane-methanol (1:0 to 95:5) to give unresolved product isomers (107 mg). The mixture was purified by chromatography on silica gel, eluting with dichloromethane-ethyl acetate-methanol (96:2:2) to give the faster-running trans isomer (47.2 mg), mixed fractions (31.2 mg) and the slower-running cis isomer (18.9 mg).

Trans isomer (Example 19): 1H-NMR (400 MHz, CD₃OD): δ 7.84 (1H, br m), 7.67 (1H, br m), 7.61 (2H, d, J 8 Hz), 7.51 (1H, d, J 8.5 Hz), 7.40 (2H, t, J 8 Hz), 7.20 (1H, t, J 8 Hz), 3.75 (2H, s), 3.04 (1H, m), 2.69 (2H, s), 2.17-2.09 (2H, m), 2.02-1.85 (4H, m) 1.79-1.69 (2H, m); UPLC-MS: 0.62 min, m/z 414 [M+H]+.

Cis isomer (Example 20): 1H-NMR (400 MHz, CD₃OD): δ 7.84 (1H, br m), 7.73-7.58 (1H, br m), 7.63 (2H, d, J 8 Hz), 7.50 (1H, d, J 8.5 Hz), 7.40 (2H, t, J 8 Hz), 7.21 (1H, t, J 8 Hz), 3.91 (2H, s), 3.03 (1H, m), 2.52 (2H, s), 2.16-2.06 (2H, m), 2.05-1.87 (4H, m) 1.76-1.66 (2H, m); UPLC-MS: 0.64 min, m/z 414 [M+H]+.

### Example 21

### (Cis)-3-phenyl-8-{5-[(trifluoromethyl)oxy]-1H-benzimidazol-2-yl}-1-oxa-3-azaspiro[4,5]decan-2-one

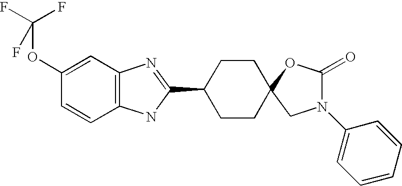

(Cis)2-Oxo-3-phenyl-1-oxa-3-azaspiro[4.5]decane-8-carbaldehyde (Intermediate 31, 50 mg, 0.193 mmol) and 4-(trifluoromethoxy)-1,2-benzenediamine (Intermediate 32, 37.0 mg, 0.193 mmol) were combined with 1,4-dioxane (2 ml) in a 4 ml vial. Air was bubbled in through a needle for ˜5 minutes then the vial was capped and warmed to 100° C. and was shaken at this temperature overnight (˜24 hours), left at room temperature for another 24 hours then dried under vacuo to give a residue that was purified by silica gel column chromatography (12+M, Biotage SP1), eluting with DCM\EtOAc 100:0 to 50:50 to afford the title compound as a brownish solid (0.034 g, 36% yield; purity 90%, 9:1 mixture of cis:trans isomers). 1H-NMR (400 MHz, CDCl₃): δ 1.73-1.92 (m, 2H), 1.95-2.41 (m, 6H), 3.01-3.23 (m, 1H), 3.82 (s, 2H), 7.08-7.25 (m, 2H), 7.33-7.71 (m, 6H), 9.60-9.98 (br s, 1H); HPLC-MS, 1: peak 1 1.969 min, m/z 432 [M+H]+ (89%); peak 2 2.110 min, m/z 432 [M+H]+ (10%).

### Example 22

### (Trans)-3-phenyl-8-{5-[(trifluoromethyl)oxy]-1H-benzimidazol-2-yl}-1-oxa-3-azaspiro[4.5]decan-2-one hydrochloride

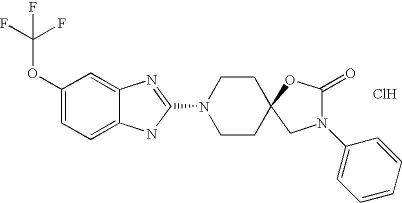

A mixture of (trans)-N-{2-amino-4-[(trifluoromethyl)oxy]phenyl}-2-oxo-3-phenyl-1-oxa-3-azaspiro[4.5]decane-8-carboxamide and (trans)-N-{2-amino-5-[(trifluoromethyl)oxy]phenyl}-2-oxo-3-phenyl-1-oxa-3-azaspiro[4.5]decane-8-carboxamide (Intermediate 34, 130 mg, 0.3 mmol), p-toluenesulfonic acid (57.1 mg, 0.3 mmol) in toluene (2 ml) and dioxane (2 ml) was stirred at 150° C. under microwave irradiation for 30 min. The reaction mixture was then purified with ion exchange SCX cartridge (2 g, Varian) washing with MeOH and eluting with 2M ammonia in methanol to afford 120 mg of crude compound. This was purified with Biotage SP1, 25M silica cartridge, eluting with a gradient of DCM/Et₂O. (Trans)-3-phenyl-8-{5-[(trifluoromethyl)oxy]-1H-benzimidazol-2-yl}-1-oxa-3-azaspiro[4.5]decan-2-one was eluted with 30% Et₂O and recovered as a colourless solid (85 mg).

The solid was then treated with 1.0 equiv. of 1M HCl in Et₂O to give the title compound as colourless solid (84 mg). 1H-NMR (400 MHz, CDCl₃): δ 1.9-2.6 (m, 8H), 3.2 (m, 1H), 4.0 (s, 2H), 7.1 (m, 1H), 7.3-7.5 (m, 3H), 7.6 (m, 1H); UPLC-MS: 0.68 min, m\z 432 [M+H]+.

### Example 23

### (Trans)-3-(2-fluorophenyl)-8-{5-[(trifluoromethyl)oxy]-1H-benzimidazol-2-yl}-1-oxa-3-azaspiro[4.5]decan-2-one hydrochloride

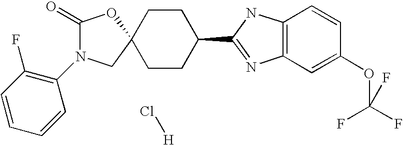

To a solution of (trans)-3-(2-fluorophenyl)-8-{5-[(trifluoromethyl)oxy]-1H-benzimidazol-2-yl}-1-oxa-3-azaspiro[4.5]decan-2-one (Example 24, 81 mg, 0.180 mmol) in DCM (1 ml) was added HCl (1.0M in Et₂O, 0.252 ml, 0.252 mmol) The solution was concentrated under vacuum to give the title compound as solid (mg 77, 88%). 1H-NMR (400 MHz, DMSO-d6): δ 1.86-2.01 (m, 4H), 2.07-2.27 (m, 4H), 3.19-3.30 (m, 1H), 3.90-3.95 (s, 2H), 7.23-7.46 (m, 4H), 7.60 (t, 1H), 7.71-7.75 (s, 1H), 7.79 (d, 1H); HPLC-MS, 1: 1.973 min, m/z 450 [M+H]+.

### Example 24

### (Trans)-3-(2-fluorophenyl)-8-{5-[(trifluoromethyl)oxy]-1H-benzimidazol-2-yl}-1-oxa-3-azaspiro[4.5]decan-2-one

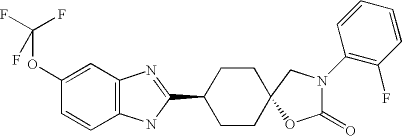

A solution of mixed (trans)-N-{2-amino-4-[(trifluoromethyl)oxy]phenyl}-3-(2-fluorophenyl)-2-oxo-1-oxa-3-azaspiro[4.5]decane-8-carboxamide and (trans)-N-{2-amino-5-[(trifluoromethyl)oxy]phenyl}-3-(6-methyl-2-pyridinyl)-2-oxo-1-oxa-3-azaspiro[4.5]decane-8-carboxamide (Intermediate 35, 0.297 mmol, 139 mg) and p-toluenesulfonic acid monohydrate (5.65 mg, 0.030 mmol) in anhydrous 1,4-dioxane (2 ml) and toluene (2 ml) was stirred at 150° C. in a microwave reactor for 30 min. The mixture was concentrated under vacuum and the residue was purified by silica gel chromatography eluting with 0-20% Et₂O\DCM to give the title compound as an oil (81 mg, 61%). 1H-NMR (400 MHz, DMSO-d6): δ 1.80-1.97 (m, 4H), 2.04-2.17 (m, 4H), 3.00-3.09 (m, 1H), 3.88-3.93 (s, 2H), 7.12 (d, 1H), 7.24-7.29 (m, 1H), 7.31-7.39 (m, 2H), 7.43-7.63 (m, 3H), 12.51 (br s, 1H); HPLC-MS, 1: 1.987 min, m/z 450 [M+H]+.

### Example 25

### (Trans)3-(4-fluorophenyl)-8-{5-[(trifluoromethyl)oxy]-1H-benzimidazol-2-yl}-1-oxa-3-azaspiro[4.5]decan-2-one hydrochloride

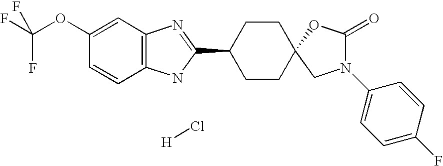

A mixture of (trans)N-{2-amino-5-[(trifluoromethyl)oxy]phenyl}-3-(4-fluorophenyl)-2-oxo-1-oxa-3-azaspiro[4.5]decane-8-carboxamide and its 5-trifluoromethyloxy regioisomer (Intermediate 41, 270 mg, 0.578 mmol) was mixed with p-toluenesulfonic acid (10.99 mg, 0.058 mmol) in 1,4-dioxane (5 ml) and toluene (5 ml). The mixture was irradiated in a microwave at 150° C. for 30 and thereafter for 20 min. The reaction mixture was transferred onto an SCX resin and eluted with MeOH followed by 2M NH₃ in MeOH. The basic fractions were evaporated and the residue was purified by silica gel chromatography eluting with DCM/MeOH 99/1 to 10/1. The fractions containing the desired product were evaporated and re-purified by silica gel chromatography eluting with DCM/Et₂O 10/1 to 1/2. Two batches were collected since some of the fractions were coloured slightly yellow: a white solid and an off-white solid. Hardly any difference between the two solids could be seen by 1H-NMR and UPLC-MS. Thus, they were combined and triturated with Et₂O (2×10 ml) to give 147.5 mg (0.328 mmol, 56.8%) of (trans)3-(4-fluorophenyl)-8-{5-[(trifluoromethyl)oxy]-1H-benzimidazol-2-yl}-1-oxa-3-azaspiro[4.5]decan-2-one, which was dissolved in DCM and treated with 1M HCl in Et₂O to form the hydrochloride salt. The solvent was stripped off and the hydrochloride salt was dried under high vacuum to give the title compound (158.7 mg, 56.5%). 1H-NMR (400 MHz, DMSO-d6): δ 7.82 (1H, d), 7.75 (1H, br s), 7.56-7.64 (2H, m), 7.41-7.48 (1H, m), 7.21-7.31 (2H, m), 3.98 (2H, s), 3.20-3.32 (1H, m), 2.17-2.27 (2H, m), 1.83-2.14 (6H, m); UPLC-MS: 0.69 min, m\z 450 [M+H]+.

### Example 26

### 2-[(Trans)-2-oxo-3-(2-pyridinyl)-1-oxa-3-azaspiro[4.5]dec-8-yl]-1H-benzimidazole-5-carbonitrile hydrochloride

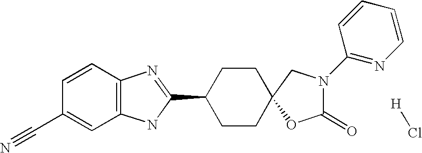

A solution of 2-[(trans)-2-oxo-3-(2-pyridinyl)-1-oxa-3-azaspiro[4.5]dec-8-yl]-1H-benzimidazole-5-carbonitrile (Example 27, 38.6 mg, 0.103 mmol) in DCM (1.5 ml), Et₂O (0.5 ml) and MeOH (0.5 ml) was treated with HCl (1.0M in Et₂O, 0.124 ml, 0.124 mmol). The mixture was concentrated under vacuum to give the title compound as a violet solid (42.9 mg). 1H-NMR (400 MHz, DMSO-d6): δ 1.85-2.05 (m, 4H), 2.1-2.25 (m, 4H), 3.2 (m, 1H), 4.04-4.13 (s, 2H), 7.11-7.20 (m, 1H), 7.75 (m, 1H), 7.8-7.9 (m, 2H), 8.15 (d, 1H), 8.25 (s, 1H), 8.4 (d, 1H); HPLC-MS, 1: 1.863 min, m/z 374 [M+H]+.

### Example 27

### 2-[(Trans)-2-oxo-3-(2-pyridinyl)-1-oxa-3-azaspiro[4.5]dec-8-yl]-1H-benzimidazole-5-carbonitrile

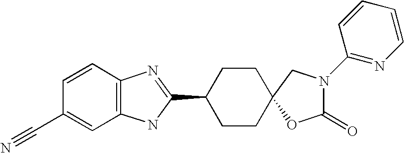

A solution of (trans)-N-(2-amino-5-cyanophenyl)-2-oxo-3-(2-pyridinyl)-1-oxa-3-azaspiro[4.5]decane-8-carboxamide (Intermediate 44, 155 mg, 0.396 mmol) and p-toluenesulfonic acid monohydrate (15.1 mg, 0.079 mmol) in 1,4-dioxane (2 ml) and toluene (2 ml) was stirred at 150° C. in a microwave reactor for 30 min. The mixture was concentrated under vacuum and the resulting residue was partitioned between DCM and saturated sodium bicarbonate solution. The combined organic extracts were dried (Na₂SO₄), filtered and concentrated under vacuum to give a residue which was purified by silica gel chromatography (eluent 0-30% Et₂O/DCM) affording the title compound as a pinkish solid (38.6 mg, 26%). 1H-NMR (400 MHz, DMSO-d6): δ 1.81-1.98 (m, 4H), 2.02-2.16 (m, 4H), 3.03-3.13 (m, 1H), 4.13 (s, 2H), 7.11-7.20 (m, 1H), 7.50-7.57 (d, 1H), 7.61-7.68 (d, 1H), 7.77-7.90 (m, 1H), 8.07 (br s, 1H), 8.12 (d, 1H), 8.36 (m, 1H), 12.84 (br s, 1H); HPLC-MS, 1: 1.859 min, m/z 374 [M+H]+.

### Example 28

### (Trans)-3-(2-pyridinyl)-8-{5-[(trifluoromethyl)oxy]-1H-benzimidazol-2-yl}-1-oxa-3-azaspiro[4.5]decan-2-one dihydrochloride

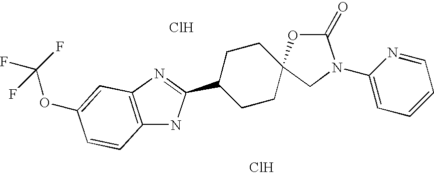

To a solution of (trans)-3-(2-pyridinyl)-8-{5-[(trifluoromethyl)oxy]-1H-benzimidazol-2-yl}-1-oxa-3-azaspiro[4.5]decan-2-one (Example 29, 77 mg) in DCM (3 ml) was added dropwise while stirring a solution of HCl (1M in Et₂O, 0.392 ml, 0.392 mmol). The solution was stirred for 30 min and then the precipitate was separated, triturated with Et₂O, concentrated under a flow of nitrogen and dried for 18 hours under high vacuum at 40° C. to give the title compound (89.0 mg, 94%). 1H-NMR (500 MHz, DMSO-d6): δ 1.81-1.93 (2H, m), 1.92-2.06 (2H, m), 2.07-2.14 (2H, m), 2.15-2.24 (2H, m), 3.22-3.36 (1H, m), 4.05-4.11 (2H, s), 7.11-7.16 (1H, m.), 7.47-7.53 (1H, m), 7.78-7.82 (1H, d), 7.80-7.86 (1H, m), 7.85-7.89 (1H, d), 8.07-8.12 (1H, d), 8.33-8.37 (1H, m); MS: m/z 433 [M+H]+.

### Example 29

### (Trans)-3-(2-pyridinyl)-8-{5-[(trifluoromethyl)oxy]-1H-benzimidazol-2-yl}-1-oxa-3-azaspiro[4.5]decan-2-one

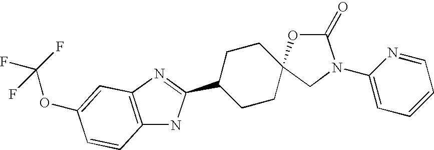

A solution of (trans)-N-{2-amino-4-[(trifluoromethyl)oxy]phenyl}-2-oxo-3-(2-pyridinyl)-1-oxa-3-azaspiro[4.5]decane-8-carboxamide (Intermediate 47, 163 mg, 0.362 mmol) and p-toluenesulfonic acid (6.89 mg, 0.036 mmol) in 1,4-dioxane (1.3 ml) and toluene (1.3 ml) was heated to 150° C. in microwave and stirred for 30 minutes for two cycles. The crude product was added to an SCX-2 ion exchange column (1 g) and was eluted with methanol (20 ml) followed by 2M ammonia/methanol (20 ml). The collected basic fractions were concentrated under vacuum and then purified on a 25+M NH column, which was eluted with DCM\Et₂O (0-70%) to give the title compound (77 mg, 47%). 1H-NMR (500 MHz, DMSO-d6): δ 1.82-1.97 (4H, m), 2.00-2.15 (4H, m), 2.97-3.10 (1H, m), 4.07-4.10 (2H, s), 7.10-7.14 (1H, dd), 7.14-7.17 (1H, dd), 7.47-7.50 (1H, s), 7.54-7.57 (1H, d), 7.83-7.87 (1H, td), 8.11-8.13 (1H, d), 8.36-8.38 (1H, dd), 12.14-13.23 (1H, br s); MS: m/z 433 [M+H]+.

### Example 30

### 2-[(Trans)-2-oxo-3-phenyl-1-oxa-3-azaspiro[4.5]dec-8-yl]-1H-benzimidazole-5-carbonitrile hydrochloride

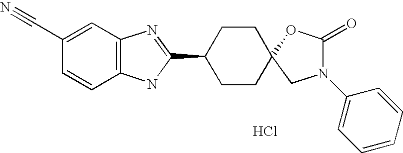

2-[(trans)-2-oxo-3-phenyl-1-oxa-3-azaspiro[4.5]dec-8-yl]-1H-benzimidazole-5-carbonitrile (Example 31, 46 mg, 0.124 mmol) was suspended in diethyl ether (1 ml). Hydrogen chloride (1M solution in Et₂O, 0.148 ml, 0.148 mmol) was added and the mixture was triturated with Et₂O (3×1 ml) to afford the title product as a pale pink solid (0.045 g, 89%). 1H-NMR (400 MHz, DMSO-d6): δ 1.78-2.28 (m, 8H), 3.08-3.26 (m, 1H), 3.98 (s, 2H), 7.07-7.19 (m, 1H), 7.35-7.46 (m, 2H), 7.54-7.82 (m, 4H), 8.16 (s, 1H); UPLC-MS: 0.63 min, m/z 373 [M+H]+.

### Example 31

### 2-[(Trans)-2-oxo-3-phenyl-1-oxa-3-azaspiro[4.5]dec-8-yl]-1H-benzimidazole-5-carbonitrile

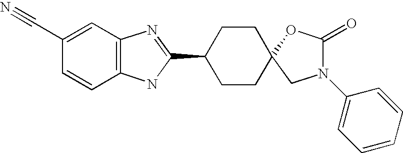

A mixture of (trans)-N-(2-amino-4-cyanophenyl)-2-oxo-3-phenyl-1-oxa-3-azaspiro[4.5]decane-8-carboxamide and (trans)-N-(2-amino-5-cyanophenyl)-2-oxo-3-phenyl-1-oxa-3-azaspiro[4.5]decane-8-carboxamide (Intermediate 48, 200 mg, quantity of mixture from previous step estimated to contain 0.3 mmol of desired compounds) was dissolved in 1,4-dioxane (1.5 ml) and toluene (1.5 ml). p-Toluenesulfonic acid (1.6 mg) was added and the mixture was heated in a microwave (150° C., 30 minutes, normal heating, 2 runs). LC-MS showed only traces of desired product. The mixture was filtered through an SCX cartridge (20 g) eluting with DCM, MeOH then 2M ammonia in MeOH. The latter fraction was dried, then diluted with DCM and partitioned with saturated. aqueous NaHCO₃ solution. The DCM extracts (3×4 ml) were combined and dried to give 93 mg of brown foam. 90 mg of this foam were dissolved in 1,4-dioxane (1.5 ml) and toluene (1.5 ml). p-Toluenesulfonic acid (2.195 mg, 0.012 mmol) was added and the mixture was heated in a microwave (150° C., 30 minutes, normal heating). LC-MS showed traces of target material. More p-toluenesulfonic acid (20 mg) was added and the mixture was heated in a microwave (150° C., 30 minutes, normal heating) then left to stand at r.t. overnight. The solvents were removed by evaporation and the mixture was partitioned between DCM and saturated. aqueous NaHCO₃ solution. The DCM extracts (3×3 ml) were combined and dried to give a residue that was purified by column chromatography (silica, 5 g) eluting with DCM:Et₂O from 100:0 to 60:40 to afford the title compound as a pink solid (0.048 g, 55%). 1H-NMR (400 MHz, CDCl₃): δ 1.97-2.12 (m, 4H), 2.12-2.23 (m, 2H), 2.29-2.42 (m, 2H), 3.07-3.17 (m, 1H), 3.88 (s, 2H), 7.14-7.19 (m, 1H), 7.40 (t, 2H) 7.51 (d, J=7.43 Hz, 1H), 7.56 (d, J=8.61 Hz, 2H), 7.80 (d, J=8.61 Hz, 1H), 8.06 (s, 1H) 9.74 (s, 1H); HPLC-MS, 2: 0.64 min, m/z 373 [M+H]+, m\z 414 [MH+MeCN]+.

### Example 32

### 2-[(Trans)-3-(4-fluorophenyl)-2-oxo-1-oxa-3-azaspiro[4.5]dec-8-yl]-1H-benzimidazole-5-carbonitrile hydrochloride

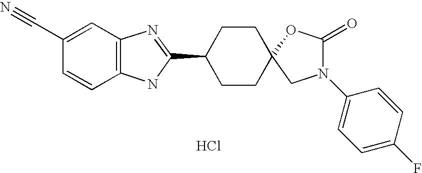

2-[(trans)-3-(4-fluorophenyl)-2-oxo-1-oxa-3-azaspiro[4.5]dec-8-yl]-1H-benzimidazole-5-carbonitrile (Example 33, 29.7 mg, 0.076 mmol) was suspended in diethyl ether (1 ml). Hydrogen chloride (1M in Et₂O, 0.091 ml, 0.091 mmol) was added and the mixture was triturated with Et₂O (3×1 ml) to afford the title compound as a pale pink solid (31.4 mg, 97%). 1H-NMR (400 MHz, DMSO-d6): δ 1.80-2.05 (m, 4H), 2.05-2.30 (m, 4H), 3.05-3.25 (m, 1H), 3.95 (s, 2H), 7.20-7.30 (m, 2H), 7.58-7.70 (m, 3H), 7.70-7.79 (m, 1H), 8.11-8.18 (m, 1H); UPLC-MS: 0.64 min, m/z 391 [M+H]+.

### Example 33

### 2-[(Trans)-3-(4-fluorophenyl)-2-oxo-1-oxa-3-azaspiro[4.5]dec-8-yl]-1H-benz-imidazole-5-carbonitrile

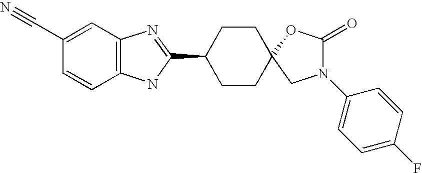

Mixture of (trans)-N-(2-amino-5-cyanophenyl)-3-(4-fluorophenyl)-2-oxo-1-oxa-3-azaspiro[4.5]decane-8-carboxamide and (trans)-N-(2-amino-4-cyanophenyl)-3-(4-fluorophenyl)-2-oxo-1-oxa-3-azaspiro[4.5]decane-8-carboxamide (Intermediate 49, 60 mg, 0.147 mmol) was dissolved in 1,4-dioxane (1.5 ml) and toluene (1.5 ml). p-Toluenesulfonic acid (14 mg, 0.074 mmol) was added and the mixture was heated in a microwave (150° C., 30 minutes, normal heating). The solvents were removed by evaporation and the mixture was partitioned between DCM/saturated aq. NaHCO₃ solution. The DCM extracts (3×3 ml) were combined and dried to give a residue that was purified by column chromatography (silica, 5 g) eluting with DCM:Et₂O from 100:0 to 80:20 to afford the title product as a pink solid (0.0297 g, 51.7%). 1H-NMR (400 MHz, CDCl₃): δ 1.95-2.15 (m, 4H), 2.15-2.25 (m, 2H), 2.25-2.45 (m, 2H), 3.05-3.20 (br m, 1H), 3.85 (s, 2H), 7.05-7.20 (m, 2H), 7.45-7.60 (m, 3H), 7.75-7.85 (m, 1H), 8.05-8.10 (m, 1H), 9.10-9.35 (br d, 1H); UPLC-MS: 0.64 min, m/z 391 [M+H]+.

### Example 34

### (Trans)-3-(2-pyridinyl)-8-[5-(trifluoromethyl)-1H-benzimidazol-2-yl]-1-oxa-3-azaspiro[4.5]decan-2-one dihydrochloride

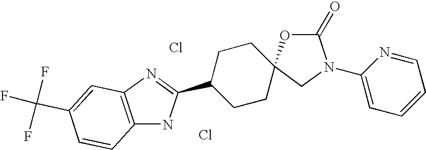

To a solution of (trans)-3-(2-pyridinyl)-8-[5-(trifluoromethyl)-1H-benzimidazol-2-yl]-1-oxa-3-azaspiro[4.5]decan-2-one (Example 35, 82.5 mg, 0.198 mmole) in DCM (2 ml) was added dropwise with stirring a solution of HCl (1M in Et₂O, 0.436 ml, 0.436 mmol). The solution was stirred for 30 mins, then the precipitate was separated, triturated with Et₂O (2 ml), concentrated under a flow of nitrogen and dried for 18 hours under high vacuum at 40° C. to give the title compound (95 mg, 98%). 1H-NMR (400 MHz, CD₃OD): δ 2.11-2.18 (m, 4H), 2.23-2.33 (m, 2H), 2.40-2.51 (m, 2H), 3.30-3.34 (m, 3H), 3.47-3.55 (m, 1H), 4.26-4.31 (m, 2H), 7.39-7.45 (m, 1H), 7.90-7.95 (m, 1H), 7.99-8.04 (m, 2H), 8.14-8.17 (m, 1H), 8.17-8.24 (m, 1H), 8.43-8.48 (m, 1H).

### Example 35

### (Trans)-3-(2-pyridinyl)-8-[5-(trifluoromethyl)-1H-benzimidazol-2-yl]-1-oxa-3-azaspiro[4.5]decan-2-one

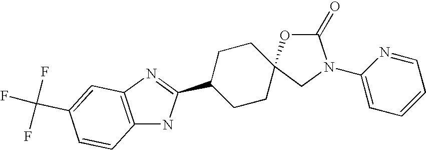

A solution of (trans)-N-[2-amino-4-(trifluoromethyl)phenyl]-2-oxo-3-(2-pyridinyl)-1-oxa-3-azaspiro[4.5]decane-8-carboxamide (Intermediate 50, 124 mg, 0.285 mmol) and p-toluenesulfonic acid (5.43 mg, 0.029 mmol) in 1,4-dioxane (1 ml) and toluene (1 ml) was heated to 150° C. in a microwave and stirred for 30 minutes for two cycles. The crude product was added to an SCX-2 ion exchange column (2 g) which was eluted with methanol (20 ml) followed by 2M ammonia/methanol (20 ml). The collected basic fractions were concentrated under vacuum and then purified on a 25+M NH column which was eluted with DCM\Et₂O (0-70%) to give the title compound (82.5 mg, 59%). 1H-NMR (500 MHz, CDCl₃): δ 1.97-2.21 (6H, m), 2.30-2.40 (2H, m), 3.10-3.12 (1H, m), 4.11-4.15 (2H, s), 7.02-7.08 (1H, m.), 7.52 (1H, m), 7.68-7.76 (1H, m), 7.84 (1H, d), 8.03 (1H, s), 8.22-8.28 (1H, m), 8.30-8.36 (1H, m), 9.10-9.23 (1H, br s); MS: m/z 417 [M+H]+.

### Example 36

### (Trans)-3-(6-methyl-2-pyridinyl)-8-{5-[(trifluoromethyl)oxy]-1H-benzimidazol-2-yl}-1-oxa-3-azaspiro[4.5]decan-2-one hydrochloride

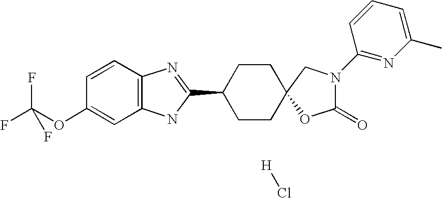

A solution of 1M HCl in Et₂O (0.172 mmol, 0.172 ml) was added to a solution of (trans)-3-(6-methyl-2-pyridinyl)-8-{5-[(trifluoromethyl)oxy]-1H-benzimidazol-2-yl}-1-oxa-3-azaspiro[4.5]decan-2-one (Example 37, 64 mg) in DCM (1.5 ml) and the mixture was shaken at room temperature for 15 min. The solvent was evaporated under vacuum and the resulting solid was triturated with Et₂O, pentane and Et₂O then dried under vacuum overnight to give the title compound (61.9 mg, 94%). 1H-NMR (400 MHz, DMSO-d6): δ 1.84-1.93 (m, 2H), 1.93-2.04 (m, 2H), 2.07-2.16 (m, 2H), 2.16-2.23 (m, 2H), 2.39-2.46 (m, 3H), 3.23-3.33 (m, 1H), 4.04-4.12 (s, 2H), 6.99-7.03 (d, 1H), 7.45-7.51 (d, 1H), 7.69-7.75 (t, 1H), 7.78-7.82 (br s, 1H), 7.83-7.88 (d, 1H), 7.89-7.95 (d, 1H); UPLC-MS: 0.69 min, m/z 447 [M+H]+.

### Example 37

### (Trans)-3-(6-methyl-2-pyridinyl)-8-{5-[(trifluoromethyl)oxy]-1H-benzimidazol-2-yl}-1-oxa-3-azaspiro[4.5]decan-2-one

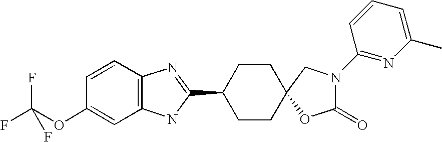

A solution of a mixture (trans)-N-{2-amino-4-[(trifluoromethyl)oxy]phenyl}-3-(6-methyl-2-pyridinyl)-2-oxo-1-oxa-3-azaspiro[4.5]decane-8-carboxamide and (trans)-N-{2-amino-5-[(trifluoromethyl)oxy]phenyl}-3-(6-methyl-2-pyridinyl)-2-oxo-1-oxa-3-azaspiro[4.5]decane-8-carboxamide (Intermediate 51, 0.211 mmol, 196 mg) and p-toluenesulfonic acid monohydrate (10.0 mg, 0.053 mmol) in anhydrous 1,4-dioxane (2 ml) and toluene (2 ml) was stirred in a microwave at 150° C. for 30 min. The mixture was concentrated under vacuum and the residue was taken into DCM and washed with a saturated sodium bicarbonate solution. The organic phase was dried (Na₂SO₄), filtered and concentrated under vacuum to obtain a residue which was purified by silica gel chromatography eluting with 0-50% Et₂O\DCM to give a residue which was re-purified by silica gel chromatography with 30% Et₂O\DCM to afford the title compound as pink solid (64 mg, 64%). 1H-NMR (400 MHz, DMSO-d6): δ 1.82-1.94 (m, 4H), 2.02-2.14 (m, 4H), 2.43-2.46 (m, 3H), 2.98-3.09 (m, 1H), 4.05 (s, 2H), 7.01 (d, 1H), 7.11-7.15 (m, 1H), 7.47-7.51 (br s, 1H), 7.55-7.59 (m, 1H), 7.72 (t, 1H), 7.92 (d, 1H), 12.55 (br s, 1H); UPLC-MS: 0.68 min, m/z 447 [M+H]+.

### Example 38

### (Trans)-3-(2-methyl-3-pyridinyl)-8-{5-[(trifluoromethyl)oxy]-1H-benzimidazol-2-yl}-1-oxa-3-azaspiro[4.5]decan-2-one dihydrochloride

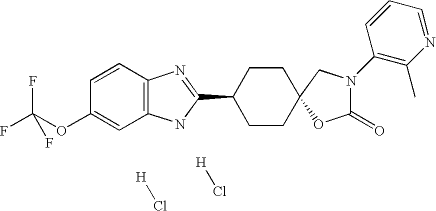

A solution of 1M HCl in Et₂O (0.242 mmol, 0.242 ml) was added to a solution of (trans)-3-(2-methyl-3-pyridinyl)-8-{5-[(trifluoromethyl)oxy]-1H-benzimidazol-2-yl}-1-oxa-3-azaspiro[4.5]decan-2-one (Example 39, 0.110 mmol, 49 mg) in DCM (1.5 ml) and the mixture was shaken for 15 minutes. The solvent was removed under vacuum to obtain the title compound as grey solid (53 mg, 93%). 1H-NMR (400 MHz, DMSO-d6): δ 1.89-2.10 (m, 4H), 2.17-2.29 (m, 4H), 2.56-2.59 (m, 3H), 3.28-3.35 (m, 1H), 3.99 (s, 2H), 7.45-7.50 (m, 1H), 7.65-7.71 (m, 1H), 7.77 (br s, 1H), 7.85 (d, 1H), 8.25-8.29 (m, 1H), 8.59-8.63 (m, 1H); UPLC-MS: 0.56 min, m/z 447 [M+H]+.

### Example 39

### (Trans)-3-(2-methyl-3-pyridinyl)-8-{5-[(trifluoromethyl)oxy]-1H-benzimidazol-2-yl}-1-oxa-3-azaspiro[4.5]decan-2-one

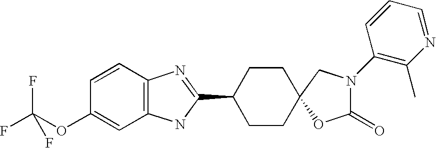

A solution of a mixture (trans)-N-{2-amino-4-[(trifluoromethyl)oxy]phenyl}-3-(2-methyl-3-pyridinyl)-2-oxo-1-oxa-3-azaspiro[4.5]decane-8-carboxamide and (trans)-N-{2-amino-5-[(trifluoromethyl)oxy]phenyl}-3-(2-methyl-3-pyridinyl)-2-oxo-1-oxa-3-azaspiro[4.5]decane-8-carboxamide (Intermediate 54, 0.233 mmol, 216 mg) and p-toluenesulfonic acid monohydrate (22.2 mg, 0.117 mmol) in anhydrous 1,4-dioxane (2 ml) and toluene (2 ml) was heated to 150° C. in microwave for 30 min. The mixture was concentrated under vacuum and the residue was taken into DCM and washed with a saturated NaHCO₃ solution. The aq. layer was backextracted with DCM and the combined organic extracts were concentrated under vacuum to give a residue. The crude was purified by NH chromatography (eluent EtOAc 100%) to afford the title compound as pinkish solid (49 mg, 47%). 1H-NMR (400 MHz, DMSO-d6): δ 1.83-1.97 (m, 4H), 2.09-2.19 (m, 4H), 2.42-2.45 (m, 3H), 3.01-3.09 (m, 1H), 3.89 (s, 2H), 7.08-7.15 (m, 1H), 7.30-7.36 (m, 1H), 7.38-7.67 (m, 2H), 7.82-7.86 (dd, 1H), 8.40-8.44 (dd, 1H), 12.48 (br s, 1H); UPLC-MS 0.54 min, m/z 447 [M+H]+.

### Example 40

### (Trans)-3-(2-pyrimidinyl)-8-{5-[(trifluoromethyl)oxy]-1H-benzimidazol-2-yl}-1-oxa-3-azaspiro[4.5]decan-2-one dihydrochloride

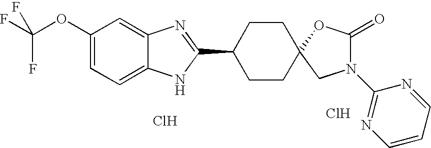

To a solution of (trans)-3-(2-pyrimidinyl)-8-{5-[(trifluoromethyl)oxy]-1H-benzimidazol-2-yl}-1-oxa-3-azaspiro[4.5]decan-2-one (Example 41, 55 mg, 0.127 mmol) in dichloromethane (DCM) (2 ml) was added 1M HCl in ether (0.317 ml, 0.317 mmol). The mixture was left to stand for 10 minutes then concentrated under a flow of nitrogen while heating at 40° C. The residue was heated for 3 hours at 40° C. under vacuum to give the title compound as a white solid (64.4 mg). 1H-NMR (400 MHz, DMSO-d6): δ 8.74 (2H, d, J 5 Hz), 7.91 (1H, d, J 9 Hz), 7.85 (1H, m), 7.56-7.52 (1H, m), 7.27 (1H, t, J 5 Hz), 4.11 (2H, s), 3.38-3.30 (1H, m), 2.27-2.18 (2H, m), 2.17-2.09 (2H, m), 2.08-1.96 (2H, m), 1.94-1.85 (2H, m); UPLC-MS 0.55 min, m/z 434 [M+H]+.

### Example 41

### (Trans)-3-(2-pyrimidinyl)-8-{5-[(trifluoromethyl)oxy]-1H-benzimidazol-2-yl}-1-oxa-3-azaspiro[4.5]decan-2-one

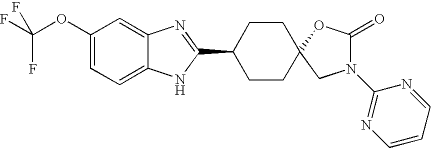

A mixture of (trans)-N-{2-amino-5-[(trifluoromethyl)oxy]phenyl}-2-oxo-3-(2-pyrimidinyl)-1-oxa-3-azaspiro[4.5]decane-8-carboxamide (Intermediate 57, 75 mg, 0.166 mmol) and p-toluenesulfonic acid (1.580 mg, 8.31 μmol) in 1,4-dioxane (1 ml) and toluene (1 ml) was heated to 150° C. in a sealed tube using microwave irradiation and stirred 30 minutes. The mixture was cooled to room temperature and quenched into saturated aqueous sodium bicarbonate solution (10 ml) and extracted with ethyl acetate (2×10 ml). The combined organic extracts were washed with water, filtered through a hydrophobic membrane and concentrated under vacuum to give the crude product (84.8 mg), which was purified by column chromatography on silica gel eluting with cyclohexane\ethyl acetate (1:0 to 1:1 to 0:1, gradient with isocratic steps), then dried under vacuum for 18 hours to give the title compound as a white solid (60.9 mg). 1H-NMR (400 MHz, CDCl₃): δ 11.45-11.07 (1H, m), 8.61 (2H, d, J 5 Hz), 7.74-7.20 (2H, m), 7.11-7.07 (1H, m), 7.06 (1H, t, J 5 Hz), 4.10 (2H, s), 3.08 (1H, septet, J 4 Hz), 2.34-2.24 (2H, m), 2.16-1.89 (6H, m); UPLC-MS: 0.55 min, m/z 434 [M+H]+.

### Example 42

### (Trans)-3-(2-fluorophenyl)-8-[5-(1H-pyrazol-1-yl)-1H-benzimidazol-2-yl]-1-oxa-3-azaspiro[4.5]decan-2-one hydrochloride

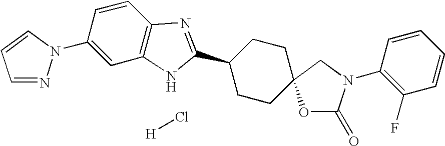

Copper(I) iodide (4.29 mg, 0.023 mmol), pyrazole (11.49 mg, 0.169 mmol), (trans)-8-(5-bromo-1H-benzimidazol-2-yl)-3-(2-fluorophenyl)-1-oxa-3-azaspiro[4.5]decan-2-one (Example 55, 50 mg, 0.113 mmol), (+\−)-trans-1,2-diaminocyclohexane (5.41 μl, 0.045 mmol) and K₂CO₃ (31.1 mg, 0.225 mmol) were stirred in dry DMSO (3 ml), under microwave irradiation at 160° C. for 30 min. The reaction mixture was cooled to r.t. Further copper(I) iodide (4.3 mg, 0.022 mmol) and (+\−)-trans-1,2-diaminocyclohexane (5.4 μl, 0.045 mmol) were added. The reaction mixture was then stirred at 160° C. under microwaves for a further 30 min, then it was cooled, diluted with ethyl acetate (30 ml) and water (50 ml). The organic layer was separated, dried over sodium sulphate, filtered and evaporated under vacuum. The crude was purified by MDAP. After evaporation, (trans)-3-(2-fluorophenyl)-8-[5-(1H-pyrazol-1-yl)-1H-benzimidazol-2-yl]-1-oxa-3-azaspiro[4.5]decan-2-one was obtained as a yellow oil (19.6 mg, 32.3%). 1H-NMR (400 MHz, CDCl₃): δ 1.60-1.71 (m, 2H), 1.97 (m, 2H), 2.14 (m, 2H), 2.27 (m, 2H), 3.03 (s, 1H), 3.87 (s, 2H), 6.50 (s, 1H), 7.11-7.22 (m, 2H), 7.50-7.61 (m, 3H), 7.69-7.76 (m, 2H), 7.86-7.94 (m, 2H), 8.25 (s, 1H); UPLC-MS: 0.56 min, m\z 432 [M+H]⁺.

To a solution of (trans)-3-(2-fluorophenyl)-8-[5-(1H-pyrazol-1-yl)-1H-benzimidazol-2-yl]-1-oxa-3-azaspiro[4.5]decan-2-one (19.6 mg, 0.036 mmol) in Et₂O (2 ml), was added 1M HCl (0.036 ml, 0.036 mmol) in diethyl ether and the reaction mixture was stirred 5 min at r.t. The solvent was evaporated under vacuum to give the title compound (21.26 mg, 100%). UPLC-MS: 0.56 min, m\z 432 [M+H]⁺.

### Example 43

### (Trans)-3-(2-fluorophenyl)-8-[5-(3-methyl-2-oxo-1-imidazolidinyl)-1H-benzimidazol-2-yl]-1-oxa-3-azaspiro[4.5]decan-2-one hydrochloride

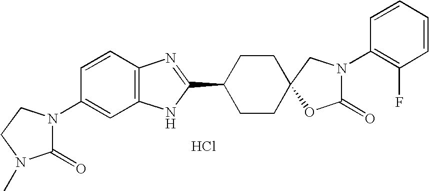

The title compound was made in a similar fashion to the preparation of Example 42 replacing pyrazole with 1-methyl-2-imidazolidinone (16.90 mg, 0.169 mmol) to give the title compound as (2.4 mg, 3.45%). 1H-NMR (400 MHz, CD₃OD): δ1.96-2.05 (m, 2H), 2.09 (d, 2H), 2.28 (d, 2H), 2.42 (br s, 2H) 2.92 (s, 3H), 3.60 (t, 2H), 3.67 (br s, 1H), 3.98 (t, 2H), 4.02 (s, 2H), 7.19-7.33 (m, 2H), 7.39 (d, 1H), 7.48-7.64 (m, 2H), 7.72 (s, 2H), 8.11 (s, 1H); UPLC-MS: 0.53 min, m\z 464 [M+H]⁺.

### Example 44

### (Trans)-3-(2-fluorophenyl)-8-[5-(1H-imidazol-1-yl)-1H-benzimidazol-2-yl]-1-oxa-3-azaspiro[4.5]decan-2-one bis(trifluoroacetate)

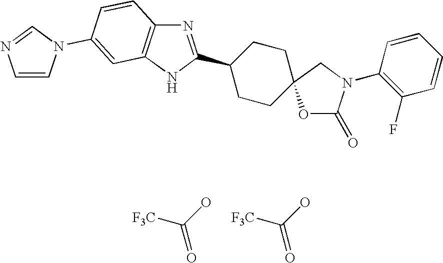

Copper(I) iodide (4.29 mg, 0.023 mmol), 1H-imidazole (11.49 mg, 0.169 mmol), (trans)-8-(5-bromo-1H-benzimidazol-2-yl)-3-(2-fluorophenyl)-1-oxa-3-azaspiro[4.5]decan-2-one (Example 55, 50 mg, 0.113 mmol), (+\−)-trans-1,2-diaminocyclohexane (5.41 μl, 0.045 mmol) and K₂CO₃ (31.1 mg, 0.225 mmol) were stirred in dry DMSO (3 ml), under microwave irradiation at 160° C. for 30 min. The reaction mixture was cooled to r.t. Further copper(I) iodide (4.3 mg, 0.022 mmol) and (+\−)-trans-1,2-diaminocyclohexane (5.4 μl, 0.045 mmol) were then added. The reaction mixture was then stirred at 160° C. under microwave irradiation for a further 30 min, then it was cooled, diluted with ethyl acetate (30 ml) and water (50 ml). The organic layer was separated, dried over sodium sulphate, filtered and evaporated under vacuum. The crude was purified by preparative HPLC R.t. 3.07 min, conditions: Column: Luna C18 (2) 100A AXIA, 100×21.2 mm, 5 μm; Mobile phase: A: H₂O+0.1% TFA; B: CH₃CN; Gradient: 10% (B) for 1 min, 10% to 50% (B) in 8 min, 50% to 100% (B) in 1 min, 100% (B) for 2 min; Flow rate: 17 ml/min; UV range: 210-350 nm; Ionization: ES+; Mass range: 100-900 amu) to afford, after evaporation of solvent, the title compound (2.0 mg, 2.16%). 1H-NMR (300 MHz, CD₃OD): δ: 1.82-2.10 (m, 6H), 2.12-2.25 (m, 2H), 3.14 (br s, 1H), 3.90 (s, 2H), 6.89-6.95 (m, 1H), 7.16 (m, 1H), 7.25 (m, 1H), 7.44 (m, 1H), 7.55 (d, 1H), 7.69 (m, 1H), 7.85 (s, 1H), 7.96 (s, 1H), 8.80 (s, 1H), 9.08 (s, 1H), 9.30 (s, 1H); HPLC-MS, 1: 3.43 min, m\z 432 [M+H]

### Example 45

### (Trans)-3-(2-fluorophenyl)-8-[5-(2-oxo-1(2H)-pyridinyl)-1H-benzimidazol-2-yl]-1-oxa-3-azaspiro[4.5]decan-2-one hydrochloride

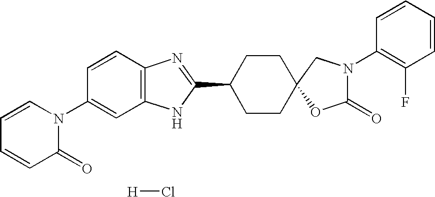

Copper(I) iodide (4.29 mg, 0.023 mmol), 2(1H)-pyridinone (16.05 mg, 0.169 mmol), (trans)-8-(5-bromo-1H-benzimidazol-2-yl)-3-(2-fluorophenyl)-1-oxa-3-azaspiro[4.5]decan-2-one (Example 55, 50 mg, 0.113 mmol), (+\−)-trans-1,2-diaminocyclohexane (5.41 μl, 0.045 mmol) and K₂CO₃ (31.1 mg, 0.225 mmol) were stirred in dry DMSO (3 ml), under microwaves at 160° C. for 30 min. Further copper(I) iodide (4.3 mg, 0.022 mmol) and (+\−)-trans-1,2-diaminocyclohexane (5.4 μl, 0.045 mmol) were then added. The reaction mixture was stirred at 160° C. under microwaves for a further 30 min, then it was cooled, diluted with ethyl acetate (30 ml) and water (50 ml). The organic layer was separated, dried over sodium sulphate, filtered and evaporated under vacuum. The crude was purified on preparative HPLC (R.t. 1.01 min, conditions: Column:Gemini C18 AXIA, 50×21 mm, 5 μm; Mobile phase: A: NH₄HCO₃ sol. 10 mM, pH 10; B: CH₃CN; Gradient: 10% to 15% (B) in 1 min, 15% to 70% (B) in 7 min, 70% to 100% (B) in 1 min, 100% (B) for 2 min; Flow rate: 17 ml/min; UV range: 210-350 nm; Ionization: ES+; Mass range: 100-900 amu). After evaporation, the residue was dissolved in 1M HCl in Et₂O (1 ml). The resulting mixture was evaporated to give the title compound (1.4 mg, 2.01%). 1H-NMR (300 MHz, CD₃OD): δ: 1.82-2.10 (m, 4H), 2.16 (m, 2H), 2.31-2-35 (m, 2H), 2.97 (br s, 1H), 3.91 (s, 2H), 6.41-6.46 (m, 2H), 6.56-6.59 (m, 2H), 7.15-7.18 (m, 2H), 7.45 (m, 1H), 7.48-7.51 (m, 3H), 7.76-7.82 (m, 2H); HPLC-MS, 1: 3.71 min, m\z 459 [M+H]⁺.

### Example 46

### (Trans)-3-(2-fluorophenyl)-8-[5-(6-oxo-1(6H)-pyridazinyl)-1H-benzimidazol-2-yl]-1-oxa-3-azaspiro[4.5]decan-2-one hydrochloride

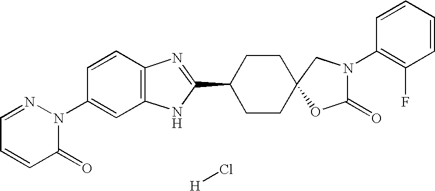

The title compound was made in a similar fashion to preparation of Example 45 replacing 2(1H)-pyridinone with 3(2H)-pyridazinone (16.22 mg, 0.169 mmol) to give the title compound (1.0 mg, 1.43%). 1H-NMR (300 MHz, CD₃OD): δ: 1.82-2.06 (m, 4H), 2.15-2.17 (m, 2H), 2.31-2-34 (m, 2H), 2.98 (br s, 1H), 3.90 (s, 2H), 7.05-7.15 (m, 2H), 7.17-7.19 (m, 2H), 7.38-7.40 (m, 2H), 7.41-7.43 (m, 1H), 7.73-7.78 (m, 2H), 7.94-7.98 (m, 2H); HPLC-MS, 1: 3.84 min, m\z 460 [M+H]⁺.

### Example 47

### (Trans)-3-(2-pyridinyl)-8-[5-(3-pyridinyloxy)-1H-benzimidazol-2-yl]-1-oxa-3-azaspiro[4.5]decan-2-one

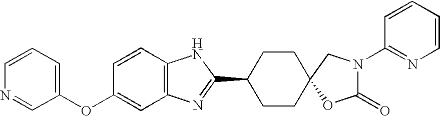

The mixture (trans)-N-[2-amino-4-(3-pyridinyloxy)phenyl]-2-oxo-3-(2-pyridinyl)-1-oxa-3-azaspiro[4.5]decane-8-carboxamide and (trans)-N-[2-amino-5-(3-pyridinyloxy)phenyl]-2-oxo-3-(2-pyridinyl)-1-oxa-3-azaspiro[4.5]decane-8-carboxamide (Intermediate 64, 103 mg, 0.202 mmol) was added to toluene (5 ml) and dioxane (5 ml). p-Toluenesulfonic acid monohydrate (38.4 mg, 0.202 mmol) was added and the reaction heated at 120° C. for 6 hours. The reaction mixture was cooled to r.t. and NaHCO₃ (sat. aqueous solution) was added (10 ml) followed by ethyl acetate (20 ml). The organic layer was separated, dried over sodium sulphate, filtered and concentrated under vacuum to give a crude oil which was purified by MDAP (method C). Collected fractions were treated with ethyl acetate (20 ml). The organic layer was separated, dried over sodium sulphate, filtered and concentrated under vacuum to give the title compound as yellow oil (56 mg, 56.5%). 1H-NMR (400 MHz, CDCl₃): 1.89-2.11 (m, 4H), 2.13-2.23 (m, 2H), 2.34 (br s, 2H), 3.07 (br s, 1H), 4.01-4.25 (s, 2H), 6.84-7.12 (m, 2H), 7.28 (m, 4H), 7.42 (s, 1H), 7.73 (t, 1H), 8.17-8.37 (m, 3H), 9.14 (s, 1H); UPLC-MS: 0.50 min, m\z 442 [M+H]⁺.

### Example 48

### (Trans)-3-(3-pyridinyl)-8-{5-[(trifluoromethyl)oxy]-1H-benzimidazol-2-yl}-1-oxa-3-azaspiro[4.5]decan-2-one hydrochloride

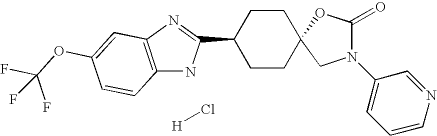

Mixture of (trans)-N-{2-amino-4-[(trifluoromethyl)oxy]phenyl}-2-oxo-3-(3-pyridinyl)-1-oxa-3-azaspiro[4.5]decane-8-carboxamide and (trans)-N-{2-amino-5-[(trifluoromethyl)oxy]phenyl}-2-oxo-3-(3-pyridinyl)-1-oxa-3-azaspiro[4.5]decane-8-carboxamide (Intermediate 67, 60 mg, 0.133 mmol) and p-toluenesulfonic acid monohydrate (25.4 mg, 0.133 mmol) were collected and suspended in 1,4-dioxane (3 ml) and toluene (1 ml). The mixture was irradiated at 150° C. for 30 min. The resulting mixture was purified by ion exchange cartridge (2 g, SCX) washing with MeOH and eluting with 2M ammonia in methanol. The resulting crude was purified by silica gel chromatography (Biotage SP1, 25+M column), using a gradient DCM\EtOH. (Trans)-3-(3-pyridinyl)-8-{5-[(trifluoromethyl)oxy]-1H-benzimidazol-2-yl}-1-oxa-3-azaspiro[4.5]decan-2-one was eluted with 10% EtOH and recovered as solid (38 mg, 88%). 1H-NMR (400 MHz, CDCl₃): δ 11.00-10.74 (m, 1H), 8.69 (br s, 1H), 8.40 (br s, 1H), 8.11 (br s, 1H), 7.74-7.59 (m, 1H), 7.43-7.26 (m, 2H), 7.16-7.10 (m, 1H), 3.83 (s, 2H).

(Trans)-3-(3-pyridinyl)-8-{5-[(trifluoromethyl)oxy]-1H-benzimidazol-2-yl}-1-oxa-3-azaspiro[4.5]decan-2-one (38 mg, 0.088 mmol) was dissolved in DCM (2 ml) and treated with 1 M HCl in Et₂O. The mixture was filtered to afford the title compound (41 mg, 65%). 1H-NMR (400 MHz, CDCl₃): δ 11.03-10.55 (m, 1H), 8.69 (br s, 1H), 8.41 (dd, 1H), 8.11 (dq, 1H), 7.77-7.52 (m, 1H), 7.46-7.26 (m, 1H), 7.36 (ddd, 1H), 7.16-7.11 (m, 1H), 3.84 (s, 2H), 3.16-3.05 (br s, 1H), 2.40-1.76 (m, 8H); UPLC-MS: 0.56 min, m\z 433 [M+H]+.

### Example 49

### (Trans)-3-(2-fluoro-3-pyridinyl)-8-{5-[(trifluoromethyl)oxy]-1H-benzimidazol-2-yl}-1-oxa-3-azaspiro[4.5]decan-2-one hydrochloride

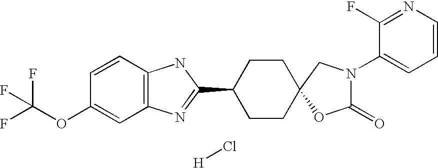

The title compound was made in a similar fashion to preparation of Example 48 replacing (trans)-N-{2-amino-4-[(trifluoromethyl)oxy]phenyl}-2-oxo-3-(3-pyridinyl)-1-oxa-3-azaspiro[4.5]decane-8-carboxamide-(trans)-N-{2-amino-5-[(trifluoromethyl)oxy]phenyl}-2-oxo-3-(3-pyridinyl)-1-oxa-3-azaspiro[4.5]decane-8-carboxamide with (trans)-N-{2-amino-4-[(trifluoromethyl)oxy]phenyl}-3-(2-fluoro-3-pyridinyl)-2-oxo-1-oxa-3-azaspiro[4.5]decane-8-carboxamide (Intermediate 70, 35 mg, 0.075 mmol) to afford the title compound as colourless solid (23 mg). 1H-NMR (400 MHz, DMSO-d6): δ 8.23-8.13 (m, 2H), 7.80-7.69 (m, 2H), 7.49-7.34 (m, 2H), 3.98 (s, 2H), 3.23 (m, 1H), 2.30-1.86 (m, 8H); UPLC-MS: 0.61 min, 455 m\z [M+H]+.

### Example 50

### (Trans)-3-(2-methylphenyl)-8-{5-[(trifluoromethyl)oxy]-1H-benzimidazol-2-yl}-1-oxa-3-azaspiro[4.5]decan-2-one hydrochloride

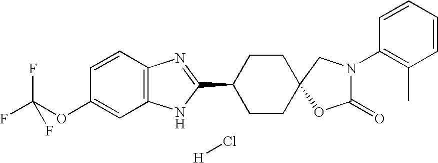

para-toluenesulfonic acid monohydrate (22.98 mg, 0.121 mmol) was added to a solution of (trans)-N-{2-amino-5-[(trifluoromethyl)oxy]phenyl}-3-(2-methylphenyl)-2-oxo-1-oxa-3-azaspiro[4.5]decane-8-carboxamide (Intermediate 73, 112 mg, 0.242 mmol) in anhydrous 1,4-dioxane (2 ml) and toluene (2 ml). The mixture was stirred at 120° C. for 1 hour then an additional amount of p-toluenesulfonic acid monohydrate (7 mg) was added and heating for further 2 hours. The mixture was diluted with DCM and washed with a saturated NaHCO₃ solution; the aqueous phase was backextracted with DCM and the combined organic extracts were dried (Na₂SO₄), filtered and concentrated in vacuo to give a residue. Purification by silica gel chromatography eluting with 30% Et₂O\DCM afforded (trans)-3-(2-methylphenyl)-8-{5-[(trifluoromethyl)oxy]-1H-benzimidazol-2-yl}-1-oxa-3-azaspiro[4.5]decan-2-one as solid (100 mg, 92%). 1H-NMR (400 MHz, DMSO-d6): δ 1.76-2.02 (m, 4H), 2.04-2.20 (m, 4H), 2.19-2.30 (m, 3H), 2.95-3.13 (m, 1H), 3.77-3.87 (m, 2H), 7.11 (d, 1H), 7.18-7.42 (m, 4H), 7.42-7.60 (m, 2H), 12.48 (br s, 1H); UPLC-MS: 0.68 min, m\z 446 [M+H]⁺.

HCl 1.0M in Et₂O (0.265 mmol, 265 μl) was added to a solution of (trans)-3-(2-methylphenyl)-8-{5-[(trifluoromethyl)oxy]-1H-benzimidazol-2-yl}-1-oxa-3-azaspiro[4.5]decan-2-one (100 mg, 0.225 mmol) in DCM and the mixture was shaken at room temperature for 1 hour. The solvent was removed under nitrogen flow affording the title compound as pale grey solid (91.4 mg, 83%). 1H-NMR (400 MHz, DMSO-d6): δ 1.84-2.09 (m, 4H), 2.11-2.30 (m, 7H), 3.20-3.34 (m, 1H), 3.84 (br s, 2H), 7.20-7.37 (m, 3H), 7.37-7.50 (m, 2H), 7.70-7.79 (m, 1H), 7.82 (d, 1H); UPLC-MS: 0.68 min, m\z 446 [M+H]⁺.

### Example 51

### (Trans)-3-(2-chlorophenyl)-8-{5-[(trifluoromethyl)oxy]-1H-benzimidazol-2-yl}-1-oxa-3-azaspiro[4.5]decan-2-one hydrochloride

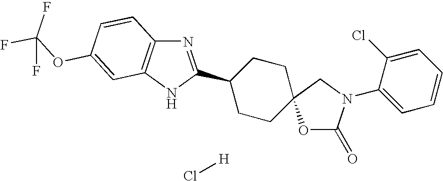

The title compound was made in a similar fashion to preparation of Example 50 replacing (trans)-N-{2-amino-5-[(trifluoromethyl)oxy]phenyl}-3-(2-methylphenyl)-2-oxo-1-oxa-3-azaspiro[4.5]decane-8-carboxamide with (trans)-N-{2-amino-5-[(trifluoromethyl)oxy]phenyl}-3-(2-chlorophenyl)-2-oxo-1-oxa-3-azaspiro[4.5]decane-8-carboxamide (Intermediate 76, 83 mg, 0.172 mmol) to give the title compound as pale grey solid (57 mg, 94%). 1H-NMR (400 MHz, DMSO-d6): δ 1.87-2.06 (m, 4H), 2.09-2.30 (m, 4H), 3.21-3.35 (m, 1H), 3.86 (br s, 2H), 7.34-7.56 (m, 3H), 7.56-7.68 (m, 2H), 7.70-7.77 (m, 1H), 7.81 (d, 1H); UPLC-MS: 0.69 min, m\z 466 [M+H]⁺.

### Example 52

### (Trans)-8-(5-bromo-1H-benzimidazol-2-yl)-3-(2-pyridinyl)-1-oxa-3-azaspiro[4.5]decan-2-one

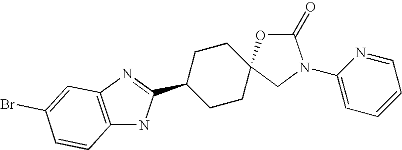

A solution of (trans)-N-(2-amino-4-bromophenyl)-2-oxo-3-(2-pyridinyl)-1-oxa-3-azaspiro[4.5]decane-8-carboxamide (Intermediate 77, 850 mg, 1.91 mmol) and p-toluenesulfonic acid monohydrate (36.3 mg, 0.191 mmol) in 1,4-dioxane (3 ml) and toluene (3 ml) was heated to 120° C. for 54 hours. The mixture was diluted with ethyl acetate and treated with a sat. NaHCO₃ solution; the aq phase was back extracted with ethyl acetate and the combined organic extracts were dried (Na₂SO₄), filtered and concentrated under vacuum to give a crude. The crude was purified by silica gel chromaphography (40+M column, eluent DCM:Et₂O 85:15) to obtain a mixture of desired compound trans-8-(5-bromo-1H-benzimidazol-2-yl)-3-(2-pyridinyl)-1-oxa-3-azaspiro[4.5]decan-2-one and (trans)-N-(2-amino-4-bromophenyl)-2-oxo-3-(2-pyridinyl)-1-oxa-3-azaspiro[4.5]decane-8-carboxamide starting material (616 mg). The mixture was dissolved in toluene (2 ml) and 1,4-dioxane (2 ml) and p-toluenesulfonic acid monohydrate was added (30 mg). The mixture was heated at 110° C. for 2 hours, then diluted with ethyl acetate and treated with a saturated NaHCO₃ solution; the aqueous phase was backextracted with ethyl acetate and combined organic extracts were dried (Na₂SO₄), filtered and concentrated under vacuum to give the title compound (500 mg, 59.5%). 1H-NMR (500 MHz, DMSO-d6): δ 1.81-1.95 (4H, m), 2.01-2.15 (4H, m), 2.96-3.06 (1H, m), 4.09 (2H, s), 7.12-7.18 (1H, dd), 7.24-7.30 (1H, d), 7.36-7.53 (1H, m), 7.59-7.75 (1H, m), 7.80-7.88 (1H, td), 8.09-8.14 (1H, d), 8.35-8.40 (1H, d), 12.34-12.70 (1H, br s); UPLC-MS: 0.83 min, m\z 427 [M+H]+

### Example 53

### (Trans)-8-[5-(3,5-dimethyl-4-isoxazolyl)-1H-benzimidazol-2-yl]-3-(2-pyridinyl)-1-oxa-3-azaspiro[4.5]decan-2-one hydrochloride

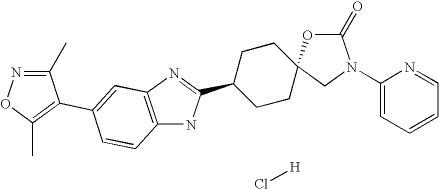

(Trans)-8-(5-bromo-1H-benzimidazol-2-yl)-3-(2-pyridinyl)-1-oxa-3-azaspiro[4.5]decan-2-one (Example 52, 51.1 mg, 0.12 mmol) was dissolved in a mixture of H₂O:DME (1:1). (3,5-Dimethyl-4-isoxazolyl)boronic acid (21.07 mg, 0.149 mmol), tetrakis(triphenylphosphine)palladium(0) (20.73 mg, 0.018 mmol) and sodium carbonate (38 mg, 0.359 mmol) were added. The mixture was stirred and heated at 100° C. in microwave oven for 30 minutes. The reaction was poured into water and extracted with DCM. The organic phase was dried on Na₂SO₄ and concentrated under vacuum. The crude was purified by flash chromatography on KP-NH column eluting with DCM\Et₂O (from 0 to 30%) to obtain (trans)-8-[5-(3,5-dimethyl-4-isoxazolyl)-1H-benzimidazol-2-yl]-3-(2-pyridinyl)-1-oxa-3-azaspiro[4.5]decan-2-one (6.6 mg, 12.4%). UPLC-MS: 0.57 min, m\z 444 [M+H]+.

To a solution of (trans)-8-[5-(3,5-dimethyl-4-isoxazolyl)-1H-benzimidazol-2-yl]-3-(2-pyridinyl)-1-oxa-3-azaspiro[4.5]decan-2-one (6.6 mg, 0.015 mmol) in DCM (2 ml) was added drop wise under stirring a 1M HCl in Et₂O (0.021 ml, 0.021 mmol). The solution was stirred at r.t. for 30 min, then the precipitate was separated, triturated with Et₂O, concentrated under nitrogen flow and dried under high vacuum at 40° C. for 18 hours to give the title compound (6.6 mg, 79%). 1H-NMR (500 MHz, DMSO-d6): δ 1.87-1.96 (2H, td), 1.96-2.06 (2H, q), 2.12-2.17 (2H, d), 2.18-2.26 (2H, m), 2.24 (3H, s), 2.42 (3H, s), 4.14 (2H, s) 7.16-7.21 (1H, dd), 7.50-7.54 (1H, d), 7.76 (1H, s), 7.83-7.90 (2H, m), 8.12-8.15 (1H, d), 8.37-8.39 (1H, d), 14.28-15.33 (1H, br s); UPLC-MS: 0.78 min, m\z 444 [M+H]+

### Example 54

### (Trans)-8-(5-bromo-1H-benzimidazol-2-yl)-3-(2-fluorophenyl)-1-oxa-3-azaspiro[4.5]decan-2-one hydrochloride

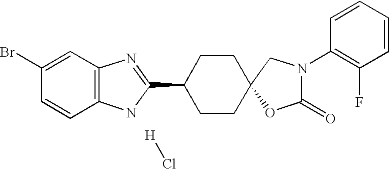

The title compound was made in a similar fashion to preparation of Example 22 using (trans)-8-(5-bromo-1H-benzimidazol-2-yl)-3-(2-fluorophenyl)-1-oxa-3-azaspiro[4.5]decan-2-one (Example 55, 15.8 mg, 0.036 mmol) to give the title compound (16.5 mg, 87%). UPLC-MS: 0.60 min, m\z 446 [M+H]⁺.

### Example 55

### (Trans)-8-(5-bromo-1H-benzimidazol-2-yl)-3-(2-fluorophenyl)-1-oxa-3-azaspiro[4.5]decan-2-one

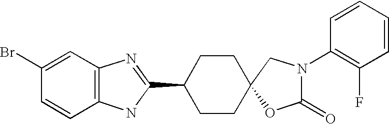

The title compound was made in a similar fashion to preparation of Example 50 replacing (trans)-N-{2-amino-5-[(trifluoromethyl)oxy]phenyl}-3-(2-methylphenyl)-2-oxo-1-oxa-3-azaspiro[4.5]decane-8-carboxamide with a mixture of (trans)-N-(2-amino-5-bromophenyl)-3-(2-fluorophenyl)-2-oxo-1-oxa-3-azaspiro[4.5]decane-8-carboxamide and (trans)-N-(2-amino-4-bromophenyl)-3-(2-fluorophenyl)-2-oxo-1-oxa-3-azaspiro[4.5]decane-8-carboxamide (Intermediate 60, 3 g, 6.49 mmol) to give the title compound as a white solid (1.98 g, 68.7%). 1H-NMR (400 MHz, CDCl₃): δ 7.55 (2H, m), 7.35 (1H, dd), 7.28 (3H, m), 7.17 (2H, m), 3.87 (2H, s), 3.03 (1H, m), 2.31 (2H, m), 2.17 (2H, m), 2.02 (3H, m); UPLC-MS: 0.60 min, m\z 446 [M+H]⁺

### Example 56

### (Trans)-8-[5-(2,3-dihydro-1,4-benzodioxin-6-yl)-1H-benzimidazol-2-yl]-3-(2-fluorophenyl)-1-oxa-3-azaspiro[4.5]decan-2-one hydrochloride

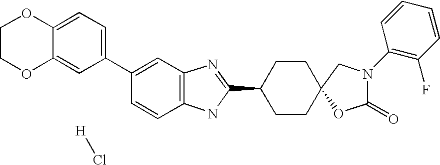

Tetrakis(triphenylphosphine)palladium(0) (1.910 mg, 1.653 μmol) was added to a solution of 1,1-dimethylethyl 5-bromo-2-[(trans)-3-(2-fluorophenyl)-2-oxo-1-oxa-3-azaspiro[4.5]dec-8-yl]-1H-benzimidazole-1-carboxylate (Intermediate 78, 30 mg, 0.055 mmol), 2,3-dihydro-1,4-benzodioxin-6-ylboronic acid (14.88 mg, 0.083 mmol) and sodium carbonate (2M solution) (0.5 ml, 1.0 mmol) in. 1,2-dimethoxyethane (5 ml). The mixture was stirred at 90° C. overnight. Additional tetrakis(triphenylphosphine)palladium(0) (2 mg) and 2,3-dihydro-1,4-benzodioxin-6-ylboronic acid (15 mg) were added and stirring was continued for further 4 hours. To the crude solution/suspension TFA (3 ml) and DCM (4 ml) were added and the reaction was stirred at room temperature overnight. The mixture was partitioned between DCM (2×3 ml) and water (4 ml). The organic layer was dried over Na₂SO₄, filtered and evaporated under reduced pressure. Purification was accomplished by silica gel chromatography on Biotage SP1 automated purification system eluting in gradient with EtOAc/Cy (2/8 to 8/2) to afford (trans)-8-[5-(2,3-dihydro-1,4-benzodioxin-6-yl)-1H-benzimidazol-2-yl]-3-(2-fluorophenyl)-1-oxa-3-azaspiro[4.5]decan-2-one (6 mg, 21.8%). 1H-NMR (400 MHz, CDCl₃): δ 7.30-7.80 (3H, m), 6.78-7.26 (5H, m), 5.29-5.34 (4H, m), 4.22-4.32 (2H, m), 4.07-4.18 (1H, m), 3.92 (1H, s), 3.22 (1H, br s), 2.09 (3H, d), 1.61-1.91 (2H, m); UPLC-MS: 0.62 min, m\z 500 [M+H]⁺.

(Trans)-8-(5-bromo-1H-benzimidazol-2-yl)-3-(2-fluorophenyl)-1-oxa-3-azaspiro[4.5]decan-2-one (15.8 mg, 0.036 mmol) was dissolved in DCM (0.5 ml) and 1M HCl in Et₂O (0.071 ml, 0.071 mmol) was added. The excess solvent and hydrochloric acid were evaporated under vacuum to give the title compound (5.5 mg, 68.3%); UPLC-MS: 0.61 min, m\z 500 [M+H]⁺.

### Examples 57 and 58

### (Trans)-3-(2-fluorophenyl)-8-{5-[2-(methyloxy)-5-pyrimidinyl]-1H-benzimidazol-2-yl}-1-oxa-3-azaspiro[4.5]decan-2-one (Example 57) and (trans)-8-(1H-benzimidazol-2-yl)-3-(2-fluorophenyl)-1-oxa-3-azaspiro[4.5]decan-2-one (Example 58)

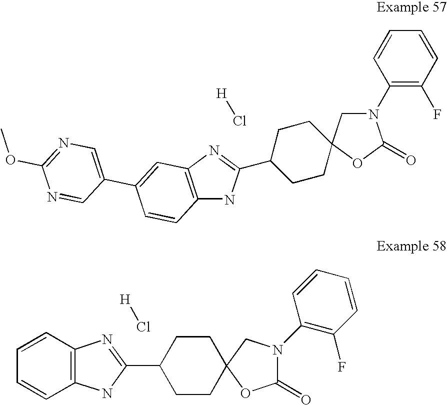

To 8-(5-bromo-1H-benzimidazol-2-yl)-3-(2-fluorophenyl)-1-oxa-3-azaspiro[4.5]decan-2-one (Example 55, 50 mg, 0.113 mmol) dissolved in 1,4-dioxane (5 ml), Pd₂ dba₃ (3.09 mg, 3.38 μmol), P(t-Bu)₃ (22.77 mg, 0.113 mmol), [2-(methyloxy)-5-pyrimidinyl]boronic acid (26.0 mg, 0.169 mmol) and cesium carbonate (44.0 mg, 0.135 mmol) were added and the solution was stirred at 90° C. for 2 hours. Then the mixture was irradiated for 15 min at 160° C. Pd₂ dba₃ (3.09 mg, 3.38 μmol), P(t-Bu)₃ (22.77 mg, 0.113 mmol), [2-(methyloxy)-5-pyrimidinyl]boronic acid (26.0 mg, 0.169 mmol) and cesium carbonate (44.0 mg, 0.135 mmol) were added and the mixture was irradiated for further 15 min at 160° C. The reaction was cooled to room temperature and the mixture partitioned between water (3 ml) and DCM (3×3 ml). The organic layer was eluted through a SCX SPE cartridge (DCM 100, 2 CV, MeOH, 3 CV, MeOH/1M Ammonia in methanol 8/2, 3 CV). Purification by chromatography on Si SPE cartridge (DCM/MeOH 100% to 9/1) afforded 3-(2-fluorophenyl)-8-{5-[2-(methyloxy)-5-pyrimidinyl]-1H-benzimidazol-2-yl}-1-oxa-3-azaspiro[4.5]decan-2-one and (trans)-8-(1H-benzimidazol-2-yl)-3-(2-fluorophenyl)-1-oxa-3-azaspiro[4.5]decan-2-one which were treated with 1.0M HCl in Et₂O (0.135 ml) to afford the title compounds 3-(2-fluorophenyl)-8-{5-[2-(methyloxy)-5-pyrimidinyl]-1H-benzimidazol-2-yl}-1-oxa-3-azaspiro[4.5]decan-2-one hydrochloride (Example 57, 9 mg, 14.11%) and (trans)-8-(1H-benzimidazol-2-yl)-3-(2-fluorophenyl)-1-oxa-3-azaspiro[4.5]decan-2-one hydrochloride (Example 58, 9 mg, 18.91%).

Example 57: 1H-NMR (400 MHz, CDCl₃): δ 8.76 (2H, s), 7.77-7.93 (1H, m), 7.46-7.61 (2H, m), 7.32-7.46 (1H, m), 7.12-7.28 (3H, m), 4.09 (3H, s), 3.88-3.91 (2H, m), 3.12 (1H, br s), 2.28-2.42 (2H, m), 2.12-2.26 (2H, m), 1.90-2.10 (4H, m); UPLC-MS: 0.56 min, m\z 474 [M+H]⁺.

Example 58: 1H-NMR (400 MHz, CDCl₃): δ 7.54 (2H, td), 7.34 (2H, dd), 7.12-7.23 (4H, m), 3.86 (3H, s), 3.03 (1H, br s), 2.25-2.38 (2H, m), 2.11-2.22 (3H, m), 1.90-2.06 (4H, m); UPLC-MS: 0.54 min, m\z 366 [M+H]⁺.

### Example 59

### 3-(2-fluorophenyl)-8-[5-(4-pyridazinyl)-1H-benzimidazol-2-yl]-1-oxa-3-azaspiro[4.5]decan-2-one

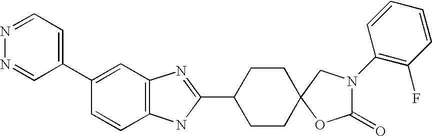

The mixture containing 1,1-dimethylethyl 5-bromo-2-[3-(2-fluorophenyl)-2-oxo-1-oxa-3-azaspiro[4.5]dec-8-yl]-1H-benzimidazole-1-carboxylate (prepared in a similar manner to Intermediate 78, 50 mg, 0.092 mmol), 4-(tributylstannanyl)pyridazine (50.9 mg, 0.138 mmol) and tetrakis(triphenylphosphine)palladium(0) (2.123 mg, 1.837 μmol) in DMSO (4 ml) was irradiated on a microwave oven (4 cycles, 15 min, 160° C.). The reaction was quenched with water (4 ml) and extracted with DCM (3×3 ml). The title compound was obtained by purification on a silica gel column on SP1 automated system eluting with DCM/MeOH 100 to 95/5 (3 mg, 6.63%). 1H-NMR (400 MHz, CDCl₃): δ 9.50-9.61 (2H, m), 9.45 (1H, dd), 9.23 (1H, dd), 7.79 (1H, dd), 7.73 (1H, d), 7.50-7.63 (3H, m), 7.05-7.26 (3H, m), 3.90 (2H, s), 3.14 (1H, br s), 2.29-2.42 (2H, m), 2.14-2.26 (2H, m), 1.96-2.11 (4H, m); UPLC-MS: 0.51 min, m\z 444 [M+H]⁺.

### Example 60

### (Trans)-3-(2-fluorophenyl)-8-[5-(5-pyrimidinyl)-1H-benzimidazol-2-yl]-1-oxa-3-azaspiro[4.5]decan-2-one

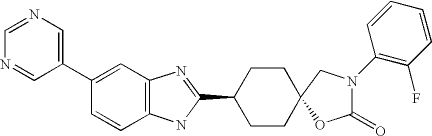

The mixture containing 1,1-dimethylethyl 5-bromo-2-[(trans)-3-(2-fluorophenyl)-2-oxo-1-oxa-3-azaspiro[4.5]dec-8-yl]-1H-benzimidazole-1-carboxylate (Intermediate 78, 50 mg, 0.092 mmol), 5-pyrimidinylboronic acid (11.38 mg, 0.092 mmol), Pd₂(dba)₃ (1.682 mg, 1.837 μmol), tri-t-butylphosphine (1 mg, 4.94 μmol) and cesium carbonate (35.9 mg, 0.110 mmol) in DMSO (4 ml) was irradiated on a microwave oven at 160° C. for 15 min (3 cycles). The mixture was quenched with water (3 ml) and extracted with DCM (3×3 ml). Purification by flash chromatography on SP1 automated system (DCM/MeOH 100% to 95/5) gave the title compound (2 mg, 5%). 1H-NMR (400 MHz, CDCl₃): δ 9.22 (1H, s), 9.01 (2H, s), 7.52-7.61 (2H, m), 7.48 (1H, dd), 7.12-7.24 (4H, m), 3.90 (2H, s), 3.13 (1H, br s), 2.36 (2H, d), 2.16-2.27 (2H, m), 1.98-2.09 (4H, m); UPLC-MS: 0.54 min, m\z 444 [M+H]⁺.

### Example 61

### (Trans)-3-(2-fluorophenyl)-8-[5-(2-pyridinyl)-1H-benzimidazol-2-yl]-1-oxa-3-azaspiro[4.5]decan-2-one

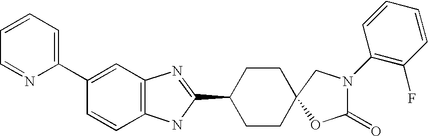

The mixture containing (trans)-8-(5-bromo-1H-benzimidazol-2-yl)-3-(2-fluorophenyl)-1-oxa-3-azaspiro[4.5]decan-2-one (Example 55, 50 mg, 0.113 mmol), 2-(tributylstannanyl)pyridine (49.7 mg, 0.135 mmol) and tetrakis(triphenylphosphine)palladium(0) (3.90 mg, 3.38 μmol) in DMSO (4 ml) was irradiated at 130° C. on a microwave oven for 30 min (3 cycles). The reaction was quenched with water (4 ml) and extracted with DCM (3×3 ml). The title compound was obtained by purification on a silica gel cartridge using Biotage SP1 automated system eluting with DCM/MeOH 100 to 95/5 (6 mg, 10.8%). 1H-NMR (400 MHz, CDCl₃): δ 8.73 (2H, d), 8.23 (2H, s), 8.06-8.02 (1H, m) 7.52-7.60 (2H, m), 7.11-7.23 (4H, m), 3.80-3.91 (2H, m), 2.91-3.06 (1H, m), 2.23-2.39 (2H, m), 2.12-2.21 (3H, m), 1.88-2.09 (4H, m); UPLC-MS: 0.56 min, m\z 443 [M+H]⁺.

### Example 62

### (Trans)-3-(2-fluorophenyl)-8-[5-(1,3-thiazol-4-yl)-1H-benzimidazol-2-yl]-1-oxa-3-azaspiro[4.5]decan-2-one

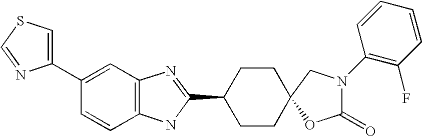

The title compound was made in a similar fashion to the preparation of Example 61 replacing 2-(tributylstannanyl)pyridine with 4-(tributylstannanyl)-1,3-thiazole (42.1 mg, 0.113 mmol) to give the title compound (3 mg, 5.3%). 1H-NMR (400 MHz, CDCl₃): δ 8.91 (1H, d), 8.17 (1H, s), 7.83 (1H, d), 7.50-7.58 (1H, m), 7.23-7.31 (3H, m), 7.10-7.22 (2H, m), 3.88 (2H, s), 2.96-3.13 (1H, m), 2.26-2.39 (2H, m), 2.10-2.24 (2H, m), 1.87-2.07 (4H, m); MS: m\z 449 [M+H]⁺.

### Example 63

### (Trans)-3-(2-fluorophenyl)-8-[5-(1,3-thiazol-2-yl)-1H-benzimidazol-2-yl]-1-oxa-3-azaspiro[4.5]decan-2-one

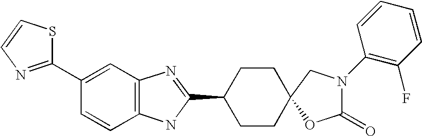

The mixture containing (trans)-8-(5-bromo-1H-benzimidazol-2-yl)-3-(2-fluorophenyl)-1-oxa-3-azaspiro[4.5]decan-2-one (Example 55, 50 mg, 0.113 mmol), tetrakis(triphenylphosphine)palladium(0) (3.90 mg, 3.38 μmol) and 2-(tributylstannanyl)-1,3-thiazole (42.1 mg, 0.113 mmol) in DMSO (4 ml) was irradiated on a microwave oven (15 min at 160° C., 2 cycles). The mixture was then stirred at 160° C. for 4 hours, quenched with water (4 ml) and extracted with DCM (3×3 ml). The organic layer was dried over Na₂SO₄, filtered and evaporated under reduced pressure. Purification by flash chromatography on SP1 system (DCM/MeOH 100 to 95/5) gave the title compound (4 mg, 8%). 1H-NMR (400 MHz, CDCl₃): δ 8.20 (1H, br s), 7.88 (1H, d), 7.56 (1H, td), 7.34 (1H, d), 7.24-7.31 (3H, m), 7.11-7.23 (2H, m), 3.88 (2H, s), 3.07 (1H, br s), 2.26-2.40 (2H, m), 2.16-2.24 (2H, m), 1.94-2.08 (4H, m); MS: m\z 449 [M+H]⁺.

### Example 64

### (Trans)-3-(2-fluorophenyl)-8-[5-(2-pyrimidinyl)-1H-benzimidazol-2-yl]-1-oxa-3-azaspiro[4.5]decan-2-one

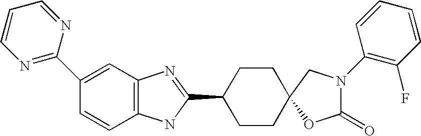

The title compound was made in a similar fashion to preparation of Example 59 replacing 4-(tributylstannanyl)-pyridazine with tributylstannanyl)pyrimidine (41.5 mg, 0.113 mmol) and 1,1-dimethylethyl 5-bromo-2-[3-(2-fluorophenyl)-2-oxo-1-oxa-3-azaspiro[4.5]dec-8-yl]-1H-benzimidazole-1-carboxylate with (trans)-8-(5-bromo-1H-benzimidazol-2-yl)-3-(2-fluorophenyl)-1-oxa-3-azaspiro[4.5]decan-2-one (Example 55, 50 mg, 0.113 mmol) to give the title compound (4 mg, 6.4%). 1H-NMR (400 MHz, CDCl₃): δ 7.93-8.07 (1H, m), 7.79-7.87 (2H, m), 7.62-7.72 (2H, m), 7.45-7.56 (1H, m), 7.20-7.29 (3H, m), 7.03-7.20 (2H, m), 3.88 (2H, s), 3.11-3.31 (1H, m), 2.21-2.39 (2H, m), 1.83-2.09 (6H, m).

### Example 65

### 8-(2-Chloro-1H-purin-8-yl)-3-phenyl-1-oxa-3-azaspiro[4.5]decan-2-one

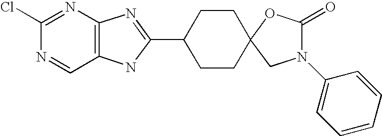

N-(5-amino-2-chloro-4-pyrimidinyl)-2-oxo-3-phenyl-1-oxa-3-azaspiro[4.5]decane-8-carboxamide-N-(4-amino-2-chloro-5-pyrimidinyl)-2-oxo-3-phenyl-1-oxa-3-azaspiro[4.5]decane-8-carboxamide (Intermediate 82, 35 mg, 0.087 mmol) was suspended in 1,4-dioxane (0.8 ml) and toluene (0.8 ml). p-Toluenesulfonic acid monohydrate (8.29 mg, 0.044 mmol) was added and the mixture was irradiated in microwave oven at 150° C. for 15 min. Solvent was removed under vacuum and the resulting residue was partitioned between saturated NaHCO₃ solution and DCM. After extraction, the organic extracts were combined and dried to afford a residue as pale yellow solid containing unreacted starting material. The residue was suspended in 1,4-dioxane (0.7 ml) and toluene (0.7 ml) and p-toluenesulfonic acid monohydrate (28.4 mg, 0.149 mmol) was added. The mixture was irradiated in microwave oven for 30 minutes at 150° C. NMP (0.1 ml) was added and the mixture was stirred at room temperature for 10 minutes up to dissolution then it was further irradiated for 30 minutes, 150° C. Solvent was removed under vacuum and the mixture was partitioned between DCM and saturated NaHCO₃ solution. After extraction with DCM (3×3 ml) the organic extracts were combined and dried to give a crude which was purified by silica gel chromatography (5 g cartridge) eluting in gradient with 1% up to 10% MeOH\DCM to give a residue. The residue was loaded into SCX cartridge (500 mg), washed with DCM then MeOH and finally eluted with 2M NH₃ in MeOH to give a residue (3.5 mg). The residue was purified by silica chromatography (1 g, elution with DCM: MeOH 98:2) affording the title compound (1.1 mg, 5.7%) as a mixture of isomers. 1H-NMR (400 MHz, CDCl₃): δ 1.74-1.93 (m, 1H), 1.93-2.11 (m, 2H), 2.11-2.49 (m, 5H), 3.08-3.35 (m, 1H), 3.78-3.93 (m, 2H), 7.10-7.24 (m, 1H), 7.34-7.49 (m, 2H), 7.50-7.63 (m, 2H), 8.83-8.99 (m, 1H); UPLC-MS: peak 1 0.61 min, m/z 384 [M+H]+; peak 2 0.62 min, m/z 384 [M+H]+.

### Examples 66 and 67

### (Trans)-3-(6-fluoro-2-pyridinyl)-8-{5-[(trifluoromethyl)oxy]-1H-benzimidazol-2-yl}-1-oxa-3-azaspiro[4.5]decan-2-one (Example 66) and (cis)-3-(6-fluoro-2-pyridinyl)-8-{5-[(trifluoromethyl)oxy]-1H-benzimidazol-2-yl}-1-oxa-3-azaspiro-[4.5]decan-2-one (Example 67)

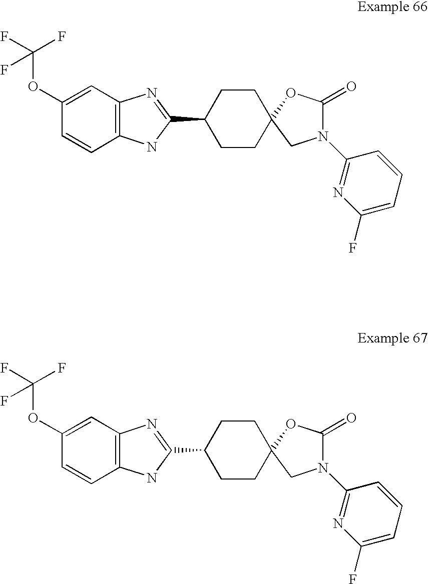

The title compounds were made in a similar fashion to preparation of Example 33 replacing the mixture of (trans)-N-(2-amino-5-cyanophenyl)-3-(4-fluorophenyl)-2-oxo-1-oxa-3-azaspiro[4.5]decane-8-carboxamide and (trans)-N-(2-amino-4-cyanophenyl)-3-(4-fluorophenyl)-2-oxo-1-oxa-3-azaspiro[4.5]decane-8-carboxamide with a mixture of N-{2-amino-4-[(trifluoromethyl)oxy]phenyl}-3-(6-fluoro-2-pyridinyl)-2-oxo-1-oxa-3-azaspiro[4.5]decane-8-carboxamide and N-{2-amino-5-[(trifluoromethyl)oxy]phenyl}-3-(6-fluoro-2-pyridinyl)-2-oxo-1-oxa-3-azaspiro[4.5]decane-8-carboxamide (Intermediate 81, 83 mg, 0.177 mmol) to give the title compounds (trans)-3-(6-fluoro-2-pyridinyl)-8-{5-[(trifluoromethyl)oxy]-1H-benzimidazol-2-yl}-1-oxa-3-azaspiro[4.5]decan-2-one (Example 66, 17.6 mg, 22%) as a pink solid and (cis)-3-(6-fluoro-2-pyridinyl)-8-{5-[(trifluoromethyl)oxy]-1H-benzimidazol-2-yl}-1-oxa-3-azaspiro[4.5]decan-2-one (Example 67, 14.8 mg, 18%) as a yellowish solid.

Example 66: 1H-NMR (500 MHz, CDCl₃): δ 1.93-2.10 (m, 4H), 2.06-2.21 (m, 2H), 2.26-2.41 (m, 2H), 3.03-3.15 (m, 1H), 4.07 (s, 2H), 6.65 (d, 1H), 7.16 (d, 1H), 7.62-7.65 (m, 1H), 7.73 (d, 1H), 7.76-7.87 (m, 1H), 8.12 (d, 1H), 8.95-9.10 (m, 1H); UPLC-MS: 0.80 min, m/z 451 [M+H]+

Example 67: 1H-NMR (400 MHz, CDCl₃): δ 1.70-1.96 (m, 2H), 2.01-2.36 (m, 6H), 3.03-3.21 (m, 1H), 4.00 (s, 2H), 6.57-6.77 (m, 1H), 7.06-7.20 (m, 1H), 7.30-7.50 (m, 1H), 7.50-7.75 (m, 1H), 7.75-7.90 (m, 1H), 8.00-8.21 (m, 1H), 9.30-9.70 (br s, 1H); UPLC-MS: 0.67 min, m/z 451 [M+H]+.

### Example 68

### (Trans)-3-(6-fluoro-2-pyridinyl)-8-{5-[(trifluoromethyl)oxy]-1H-benzimidazol-2-yl}-1-oxa-3-azaspiro[4.5]decan-2-one hydrochloride

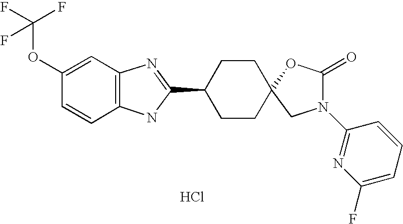

(Trans)-3-(6-fluoro-2-pyridinyl)-8-{5-[(trifluoromethyl)oxy]-1H-benzimidazol-2-yl}-1-oxa-3-azaspiro[4.5]decan-2-one (Example 66, 15.3 mg, 0.034 mmol) was suspended in diethyl ether (1 ml). and 1.0M HCl in Et₂O (0.041 ml, 0.041 mmol) was added and the mixture was triturated with Et₂O (3×1 ml) to afford the title compound as a greyish solid (16.1 mg, 93%). 1H-NMR (400 MHz, DMSO-d6): δ 1.79-2.02 (m, 4H), 2.02-2.25 (m, 4H), 3.10-3.34 (m, 1H), 3.51-3.96 (br s, 1H), 4.05 (s, 2H), 6.86-6.97 (m, 1H), 7.34-7.50 (m, 1H), 7.68-7.88 (m, 2H), 7.96-8.12 (m, 2H); UPLC-MS: 0.70 min, m/z 451 [M+H]+.

### Example 69

### 3-(6-fluoro-2-pyridinyl)-8-[5-(trifluoromethyl)-1H-benzimidazol-2-yl]-1-oxa-3-azaspiro[4.5]decan-2-one

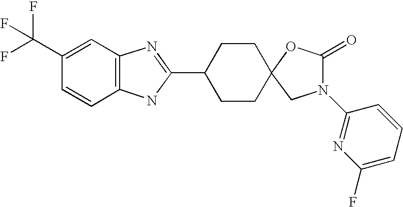

The title compound was made in a similar fashion to preparation of Example 21 replacing (cis)-2-oxo-3-phenyl-1-oxa-3-azaspiro[4.5]decane-8-carbaldehyde with 3-(6-fluoro-2-pyridinyl)-2-oxo-1-oxa-3-azaspiro[4.5]decane-8-carbaldehyde (Intermediate 84, 29 mg, 0.104 mmol) and (trifluoromethoxy)-1,2-benzenediamine with 4-(trifluoromethyl)-1,2-benzenediamine (commercially available, 18.36 mg, 0.104 mmol) to afford two batches of the title compound of different isomeric ratios:

Batch 1: ˜35:65 mixture of isomer 1 & 2 (according to NMR analysis) (5.9 mg, 11.6%). 1H-NMR (400 MHz, CDCl₃): δ 1.77-1.94 (m, 2H), 1.94-2.10 (m, 2H), 2.09-2.30 (m, 4H), 2.30-2.45 (m, 1H), 3.06-3.26 (m, 1H), 4.01 major isomer, 4.06 minor isomer (m, 2H), 6.62-6.73 (m, 1H), 7.44-7.58 (m, 2H), 7.74-7.90 (m, 2H), 8.06-8.18 (m, 2H); UPLC-MS: peak 1 0.69 min, m/z 435 [M+H]+; peak 2 0.72 min, m/z 435 [M+H]+.

Batch 2: ˜8:92 mixture of isomer 1 & 2 (5.0 mg, 8.7%). 1H-NMR (400 MHz, CDCl₃): δ 1.73-1.97 (m, 2H), 2.03-2.47 (m, 6H), 3.04-3.27 (m, 1H), 4.02 (s, 2H), 6.61-6.74 (m, 1H), 7.47-7.60 (m, 2H), 7.70-7.90 (m, 2H), 7.98-8.18 (m, 2H); UPLC-MS: 0.70 min, m/z 435 [M+H]+.

### Example 70

### 3-(2-pyridinyl)-8-[6-(trifluoromethyl)-1H-imidazo[4,5-c]pyridin-2-yl]-1-oxa-3-azaspiro[4.5]decan-2-one

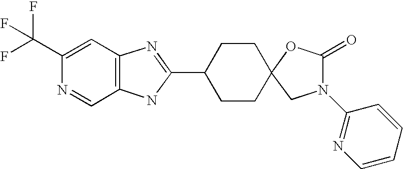

Crude 3-(2-pyridinyl)-8-[6-(trifluoromethyl)-1H-imidazo[4,5-c]pyridin-2-yl]-1-oxa-3-azaspiro[4.5]decan-2-one (Intermediate 85, 32 mg, 0.073 mmol) was suspended in 1,4-dioxane (0.5 ml) and toluene (0.5 ml). p-Toluenesulfonic acid monohydrate (13.98 mg, 0.073 mmol) was added and the mixture was irradiated at 150° C. for 30 minutes in microwave oven (3 cycles of addition and irradiation). The mixture was concentrated under vacuum and the resulting residue was partitioned between DCM (2 ml) and saturated NaHCO₃ aqueous solution (2 ml). The aqueous phase was extracted with DCM (2×2 ml), combined organic extracts were dried (Na₂SO₄) and concentrated under vacuum to give a solid which was purified by MDAP to afford the title compound as ˜30:70 mixture of isomers (relative chemical shifts of CH2N methine groups suggest major isomer is trans isomer) (2.5 mg, 7%, white solid). 1H-NMR (400 MHz, CDCl₃): δ 1.78-1.96 (m, 2H), 1.96-2.49 (m, 6H), 3.10-3.33 (m, 1H), 4.09 major isomer (s, 2H), 4.13 minor isomer (s, 2H), 7.01-7.13 (m, 1H), 7.68-7.81 (m, 1H), 7.81-8.17 (br s, 1H), 8.21-8.30 (m, 1H), 8.30-8.42 (m, 1H), 8.96-9.23 (m, 1H); UPLC-MS: peak 1 0.63 min, m/z 418 [M+H]+ (minor compound, isomer 1); peak 2 0.64 min, m/z 418 [M+H]+ (major compound, isomer 2)

### Example 71

### 3-(2-pyridinyl)-8-[6-(trifluoromethyl)-1H-imidazo[4,5-b]pyridin-2-yl]-1-oxa-3-azaspiro[4,5]decan-2-one

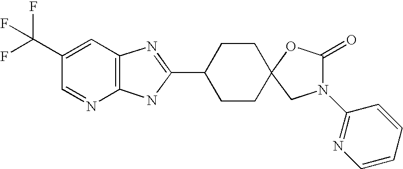

The mixture N-[2-amino-5-(trifluoromethyl)-3-pyridinyl]-2-oxo-3-(2-pyridinyl)-1-oxa-3-azaspiro[4.5]decane-8-carboxamide and N-[3-amino-5-(trifluoromethyl)-2-pyridinyl]-2-oxo-3-(2-pyridinyl)-1-oxa-3-azaspiro[4.5]decane-8-carboxamide (Intermediate 86, 54 mg, 0.124 mmol) was suspended in 1,4-dioxane (1 ml) and toluene (1 ml). p-Toluenesulfonic acid monohydrate (28.3 mg, 0.149 mmol) was added and the mixture was stirred at 120° C. under nitrogen atmosphere for 5 hours then left standing at rt overnight. Additional p-toluenesulfonic acid monohydrate (15 mg, 0.079 mmol) was added and the mixture was stirred at 120° C. for 3 hours, then cooled to r.t. and concentrated under vacuum to give a solid. The solid was partitioned between DCM (2 ml) and saturated NaHCO₃ aqueous solution (2 ml). The aqueous phase was extracted with DCM (2×2 ml), the organic extracts were combined, dried (Na₂SO₄) and concentrated to give a whitish solid (53 mg) containing unreacted starting material as determined by LC-MS. The crude material (52 mg, 0.120 mmol) was dissolved in 1,4-dioxane (1 ml) and toluene (1 ml); p-toluenesulfonic acid monohydrate (27.3 mg, 0.143 mmol) was added and the mixture was irradiated in microwave oven at 150° C., for 40 minutes followed by another addition of reagents (p-toluenesulfonic acid monohydrate 27.3 mg, 0.143 mmol and 1,4-dioxane 1 ml) and a microwave cycle (150° C., normal heating, 30 minutes). The mixture was concentrated under vacuum, the residue was partitioned between DCM (2 ml) and saturated NaHCO₃ aqueous solution (2 ml). The aqueous phase was back extracted with DCM (2×2 ml) and the combined organic extracts were dried (Na₂SO₄) and concentrated to give a crude. The crude was purified by MDAP to afford the title compound as a mixture of isomers (10 mg; 20%, white solid). 1H-NMR (400 MHz, CDCl₃): δ 1.78-1.96 (m, 1H), 2.00-2.49 (m, 7H), 3.11-3.34 (m, 1H), 4.08 major isomer (s, 2H), 4.13 minor isomer (s, 2H), 7.02-7.16 (m, 1H), 7.69-7.81 (m, 1H), 8.21-8.31 (m, 2H), 8.31-8.41 (m, 1H), 8.59-8.72 (m, 1H); HPLC-MS, 1: 1.541 min; m/z 418, [M+H]+.

### EXAMPLE

### In Vitro Profile

The in vitro assessment of the NPY Y5 antagonist compounds used different assay systems to determine the potency and affinities against the NPY Y5 receptor.

The affinities of the compounds of the invention for the NPY Y5 receptor may be determined by the binding assays described below. Such affinity is typically calculated from the IC₅₀ obtained in competition experiments as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22: 3099, 1973). In the context of the present invention pKi values (corresponding to the antilogarithm of Ki) are used instead of Ki; pKi results are only estimated to be accurate to about 0.3-0.5.

The functional activity of the compounds of the invention for the NPY Y5 receptor may be determined by the FLIPR/Ca²⁺ assay as described below. Such potency is typically calculated from the IC₅₀ obtained in FLIPR experiments as the concentration of a compound necessary to decrease 50% of the calcium release following cells exposure to a concentration of PYY eliciting 80% response (i.e. EC₈₀), and is reported as a “fKᵢ” value calculated by the following equation:

$${fK}_{i} = \frac{{IC}_{50}}{1 + EC 80 / EC 50}$$

where EC₈₀ and EC₅₀ corresponding to the agonist (PYY) concentrations that eliciting 80% and 50% response, respectively (corresponding to the Cheng and Prusoff equation). In the context of the present invention pfKi values (corresponding to the antilogarithm of fKi) are used instead of fKi; pfKi results are only estimated to be accurate to about 0.3-0.5.

### Functional Activity at Recombinant Human NPY Y5 Receptor

The functional activity at the human NPY Y5 receptor stably expressed in HEK293 cells was assessed using FLIPR/Ca²⁺ methodology (cell line name: HEK 293 signal-hNPY Y5/G16z49). The assay is configured to re-direct receptor-mediated signalling to the calcium release from intracellular stores via the promiscuous Gα16z49 protein. PYY (peptide YY) is an endogenous agonist and can activate the receptor, thereupon causing an increase in the level of calcium in the cells sensed by Fluo4-AM and measured by FLIPR. Antagonist effects are monitored by the blockade or decrease in calcium release once cells co-expressing hNPY Y5 receptor and Gα16z49 are exposed to a concentration of PYY eliciting 80% response (i.e. EC₈₀). A non-linear, 4 parameter logistic curve-fit of the data generated pIC₅₀ value. Applying the Cheng-Prusoff equation to antagonist concentration-response for inhibition of fixed PYY concentration yielded the fpKi values.

Cells are cultured in DMEM/F12 supplemented with 10% FBS, 2 mM Glutamine, 200 μg/mL hygromycin B and 500 μg/mL G418. The day before a FLIPR experiment, cells are plated out into 384-well Poly-D-Lysine coated FLIPR plates at a density of 200′000 cells/mL corrects to give 10′000 cells per 50 μL per well using medium without antibiotics.

On the day of experiment, cells are washed with an assay buffer containing 20 mM HEPES/NaOH, 145 mM NaCl, 5 mM KCl, 1 mM MgCl₂, 2 mM CaCl₂, 1 g/L D-glucose and 2.5 mM probenecid, pH 7.3 and loaded with 2 μM Fluo-4 AM for 60 min at 37° C. and 5% CO₂. The excess of dye solution is removed by washing cells with buffer. Compound solutions, prepared by serially diluting compounds in neat DMSO and then a final 1:50 dilution step in assay buffer added with 0.05% pluronic acid, are added and incubated with the loaded cells for 30 min at 37° C. and 5% CO₂.

Cells are then put in the FLIPR for the stimulus addition corresponding to a concentration of PYY eliciting 80% of the response. The response of cells to the agonist is fast and measured for 2 min after PYY addition.

### Binding Affinities at Human and Rat NPY Y5 Receptors

The assays used to measure compound affinity to human and rat NPY Y5 receptors were binding assays using Scintillation Proximity Assay (SPA) technology. The SPA involves the coupling of cell membrane fragments, via their glycosylated residues, to the wheat germ agglutinin (WGA) present on the surface of SPA beads. This coupling mechanism immobilises receptors in close proximity to the scintillant within the SPA beads and binding to the receptors of a radiolabelled ligand can thus be measured directly without the need to separate bound from free ligand.

Binding experiments are carried out in 384-well plates. The assay buffer contains 50 mM HEPES/NaOH pH 7.4, 1 mM MgCl₂, 2.5 mM CaCl2 and 0.05% pluronic acid. Specific binding is defined as the portion of [125I]-porcinePYY that is displaceable by 1 μM human PYY. A non-linear, 4 parameter logistic curve-fit of the data generated pIC₅₀ and pKi values.

### 125I-PYY Binding on Human NPY Y5 BacMam Membranes

Competition experiments are carried out in 384-well white with clear bottom plates in a final volume of 50 μl. PVT-WGA beads and membranes (prepared from HEK293F G0 cells) are diluted in assay buffer to have 2.5 mg/ml and 50 μg/ml, respectively and precoupled at 4° C. for 60 min. [125I]-PYY is added to the membrane-beads mix to achieve a concentration of 20 μM. 50 μl of the SPA mix is added to each well containing 0.5 μl compound solution. Compound solutions are prepared by serially diluting compounds in neat DMSO. The incubation lasted 3 hours at room temperature under gentle shaking. Then plates are left overnight at room temperature to allow the beads to settle and bound radioactivity is measured using Trilux MicroBeta.

### 125I-PYY Binding on Rat NPY Y5 BacMam Membranes

Competition experiments are carried out in 384-well white plates in a final volume of 30 μl. WGA-Polystyrene LEADseeker imaging beads and membranes (prepared from HEK293F G0 cells), are diluted in assay buffer to have 2.5 mg/ml and 30 μg/ml, respectively and precoupled at 4° C. for 60 min. [125I]-PYY is added to the membrane-beads mix to achieve a concentration of 75 μM. 30 μl of the SPA mix is added to each well containing 0.3 μl compounds solution. Compound solutions are prepared by serially diluting compounds in neat DMSO. The incubation lasted 3 hours at room temperature under gentle shaking. Then plates are left overnight at room temperature and bound radioactivity is measured using ViewLux.

All the compounds of formula (I) are believed to bind the NPY Y5 receptor. Preferred compounds show pKi comprised between 6 and 10 and fpKi comprised between 6 and 11 towards NPY Y5 receptor.

What is claimed is:

1. A compound of formula (I), or a pharmaceutically acceptable salt thereof, 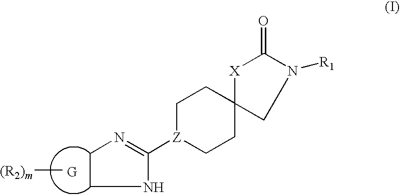
R₁ may be C₁-C₄ alkyl, aryl or heteroaryl, which may be substituted by one or more: halogen. C₁-C₄ alkyl, C₁-C₄ alkoxy, C₁-C₄ haloalkyl, C₁-C₄ haloalkoxy, cyano;
R₂ may be halogen, C₁-C₄ alkyl, C₁-C₄ alkoxy, C₁-C₄ haloalkyl, C₁-C₄ haloalkoxy, cyano, nitro; or aryl, heteroaryl or heterocycle, which may be substituted by one or more: halogen, C₁-C₄ alkyl, C₁-C₄ alkoxy, C₁-C₄ haloalkyl, C₁-C₄ haloalkoxy, cyano; or R₂ may correspond to —O—R₃;
R₃ is a 6-membered aromatic carbocyclic ring which may contain 1 or 2 nitrogen
X is carbon or oxygen;
Z is carbon or nitrogen;
G is a fused 6-membered aromatic carbocyclic ring which may contain 1 or 2 nitrogen;
m may be 0 or an integer ranging from 1 to 4.

2. A compound according to claim 1 wherein said compound is of formula (Ia)′, or a pharmaceutically acceptable salt thereof, 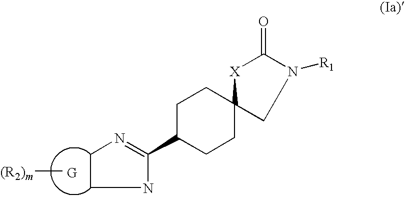
in which R₁, R₂, X, G, Z, and m are defined as in claim 1.

3. A compound according to claim 1 wherein said compound is of formula (Ib)′, or a pharmaceutically acceptable salt thereof, 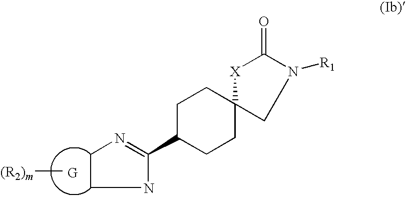
in which R₁, R₂, X, G, Z, and m are defined as in claim 1.

4. A compound or a pharmaceutically acceptable salt thereof according to claim 1 selected from a group consisting of:
8-(1H-benzimidazol-2-yl)-3-phenyl-1-oxa-3,8-diazaspiro[4.5]decan-2-one);
3-phenyl-8-[5-(trifluoromethyl)-1H-benzimidazol-2-yl]-1-oxa-3,8-diazaspiro-[4.5]decan-2-one;
(cis)-3-phenyl-8-[5-(trifluoromethyl)-1H-benzimidazol-2-yl]-1-oxa-3-azaspiro[4.5]decan-2-one;
(trans)-3-phenyl-8-[5-(trifluoromethyl)-1H-benzimidazol-2-yl]-1-oxa-3-azaspiro[4.5]decan-2-one;
8-(5-bromo-1H-benzimidazol-2-yl)-3-phenyl-1-oxa-3,8-diazaspiro[4.5]decan-2-one;
8-(5,6-dichloro-1H-benzimidazol-2-yl)-3-phenyl-1-oxa-3,8-diazaspiro[4.5]decan-2-one;
8-(5-fluoro-1H-benzimidazol-2-yl)-3-phenyl-1-oxa-3,8-diazaspiro[4.5]decan-2-one;
3-(3,4-dichlorophenyl)-8-[5-(trifluoromethyl)-1H-benzimidazol-2-yl]-1-oxa-3,8-diazaspiro[4.5]decan-2-one;
8-[5-(4-fluorophenyl)-1H-benzimidazol-2-yl]-3-phenyl-1-oxa-3,8-diazaspiro[4.5]decan-2-one;
8-(5-chloro-1H-benzimidazol-2-yl)-3-phenyl-1-oxa-3,8-diazaspiro[4.5]decan-2-one;
8-[5-(methyloxy)-1H-benzimidazol-2-yl]-3-phenyl-1-oxa-3,8-diazaspiro[4.5]decan-2-one;
3-methyl-8-[5-(trifluoromethyl)-1H-benzimidazol-2-yl]-1-oxa-3,8-diazaspiro[4.5]decan-2-one;
8-(5-chloro-1H-benzimidazol-2-yl)-3-methyl-1-oxa-3,8-diazaspiro[4.5]decan-2-one;
3-methyl-8-[5-(methyloxy)-1H-benzimidazol-2-yl]-1-oxa-3,8-diazaspiro[4.5]decan-2-one;
3-phenyl-8-{5-[(trifluoromethyl)oxy]-1H-benzimidazol-2-yl}-1-oxa-3,8-diazaspiro[4.5]decan-2-one;
(trans)-2-phenyl-8-[5-(trifluoromethyl)-1H-benzimidazol-2-yl]-2-azaspiro[4.5]decan-3-one;
(cis)-2-phenyl-8-[5-(trifluoromethyl)-1H-benzimidazol-2-yl]-2-azaspiro[4.5]decan-3-one;
(cis)-3-phenyl-8-{5-[(trifluoromethyl)oxy]-1H-benzimidazol-2-yl}-1-oxa-3-azaspiro[4.5]decan-2-one;
(trans)-3-phenyl-8-{5-[(trifluoromethyl)oxy]-1H-benzimidazol-2-yl}-1-oxa-3-azaspiro[4.5]decan-2-one;
(trans)-3-(2-fluorophenyl)-8-{5-[(trifluoromethyl)oxy]-1H-benzimidazol-2-yl}-1-oxa-3-azaspiro[4.5]decan-2-one;
(trans)3-(4-fluorophenyl)-8-{5-[(trifluoromethyl)oxy]-1H-benzimidazol-2-yl}-1-oxa-3-azaspiro[4.5]decan-2-one;
2-[(trans)-2-oxo-3-(2-pyridinyl)-1-oxa-3-azaspiro[4.5]dec-8-yl]-1H-benzimidazole-5-carbonitrile;
(trans)-3-(2-pyridinyl)-8-{5-[(trifluoromethyl)oxy]-1H-benzimidazol-2-yl}-1-oxa-3-azaspiro[4.5]decan-2-one;
2-[(trans)-2-oxo-3-phenyl-1-oxa-3-azaspiro[4.5]dec-8-yl]-1H-benzimidazole-5-carbonitrile;
2-[(trans)-3-(4-fluorophenyl)-2-oxo-1-oxa-3-azaspiro[4.5]dec-8-yl]-1H-benzimidazole-5-carbonitrile;
(trans)-3-(2-pyridinyl)-8-[5-(trifluoromethyl)-1H-benzimidazol-2-yl]-1-oxa-3-azaspiro[4.5]decan-2-one;
(trans)-3-(6-methyl-2-pyridinyl)-8-{5-[(trifluoromethyl)oxy]-1H-benzimidazol-2-yl}-1-oxa-3-azaspiro[4.5]decan-2-one;
(trans)-3-(2-methyl-3-pyridinyl)-8-{5-[(trifluoromethyl)oxy]-1H-benzimidazol-2-yl}-1-oxa-3-azaspiro[4.5]decan-2-one;
(trans)-3-(2-pyrimidinyl)-8-{5-[(trifluoromethyl)oxy]-1H-benzimidazol-2-yl}-1-oxa-3-azaspiro[4.5]decan-2-one;
(trans)-3-(2-fluorophenyl)-8-[5-(1H-pyrazol-1-yl)-1H-benzimidazol-2-yl]-1-oxa-3-azaspiro[4.5]decan-2-one;
(trans)-3-(2-fluorophenyl)-8-[5-(3-methyl-2-oxo-1-imidazolidinyl)-1H-benzimidazol-2-yl]-1-oxa-3-azaspiro[4.5]decan-2-one;
(trans)-3-(2-fluorophenyl)-8-[5-(1H-imidazol-1-yl)-1H-benzimidazol-2-yl]-1-oxa-3-azaspiro[4.5]decan-2-one;
(trans)-3-(2-fluorophenyl)-8-[5-(2-oxo-1(2H)-pyridinyl)-1H-benzimidazol-2-yl]-1-oxa-3-azaspiro[4.5]decan-2-one;
(trans)-3-(2-fluorophenyl)-8-[5-(6-oxo-1(6H)-pyridazinyl)-1H-benzimidazol-2-yl]-1-oxa-3-azaspiro[4.5]decan-2-one;
(trans)-3-(2-pyridinyl)-8-[5-(3-pyridinyloxy)-1H-benzimidazol-2-yl]-1-oxa-3-azaspiro[4.5]decan-2-one;
(trans)-3-(3-pyridinyl)-8-{5-[(trifluoromethyl)oxy]-1H-benzimidazol-2-yl}-1-oxa-3-azaspiro[4.5]decan-2-one;
(trans)-3-(2-fluoro-3-pyridinyl)-8-{5-[(trifluoromethyl)oxy]-1H-benzimidazol-2-yl}-1-oxa-3-azaspiro[4.5]decan-2-one;
(trans)-3-(2-methylphenyl)-8-{5-[(trifluoromethyl)oxy]-1H-benzimidazol-2-yl}-1-oxa-3-azaspiro[4.5]decan-2-one;
(trans)-3-(2-chlorophenyl)-8-{5-[(trifluoromethyl)oxy]-1H-benzimidazol-2-yl}-1-oxa-3-azaspiro[4.5]decan-2-one;
(trans)-8-(5-bromo-1H-benzimidazol-2-yl)-3-(2-pyridinyl)-1-oxa-3-azaspiro[4.5]decan-2-one;
(trans)-8-[5-(3,5-dimethyl-4-isoxazolyl)-1H-benzimidazol-2-yl]-3-(2-pyridinyl)-1-oxa-3-azaspiro[4.5]decan-2-one;
(trans)-8-(5-bromo-1H-benzimidazol-2-yl)-3-(2-fluorophenyl)-1-oxa-3-azaspiro[4.5]decan-2-one;
(trans)-8-[5-(2,3-dihydro-1,4-benzodioxin-6-yl)-1H-benzimidazol-2-yl]-3-(2-fluorophenyl)-1-oxa-3-azaspiro[4.5]decan-2-one;
(trans)-3-(2-fluorophenyl)-8-{5-[2-(methyloxy)-5-pyrimidinyl]-1H-benzimidazol-2-yl}-1-oxa-3-azaspiro[4.5]decan-2-one;
(trans)-8-(1H-benzimidazol-2-yl)-3-(2-fluorophenyl)-1-oxa-3-azaspiro[4.5]decan-2-one;
3-(2-fluorophenyl)-8-[5-(4-pyridazinyl)-1H-benzimidazol-2-yl]-1-oxa-3-azaspiro[4.5]decan-2-one;
(trans)-3-(2-fluorophenyl)-8-[5-(5-pyrimidinyl)-1H-benzimidazol-2-yl]-1-oxa-3-azaspiro[4.5]decan-2-one;
(trans)-3-(2-fluorophenyl)-8-[5-(2-pyridinyl)-1H-benzimidazol-2-yl]-1-oxa-3-azaspiro[4.5]decan-2-one;
(trans)-3-(2-fluorophenyl)-8-[5-(1,3-thiazol-4-yl)-1H-benzimidazol-2-yl]-1-oxa-3-azaspiro[4.5]decan-2-one;
(trans)-3-(2-fluorophenyl)-8-[5-(1,3-thiazol-2-yl)-1H-benzimidazol-2-yl]-1-oxa-3-azaspiro[4.5]decan-2-one;
(trans)-3-(2-fluorophenyl)-8-[5-(2-pyrimidinyl)-1H-benzimidazol-2-yl]-1-oxa-3-azaspiro[4.5]decan-2-one;
8-(2-Chloro-1H-purin-8-yl)-3-phenyl-1-oxa-3-azaspiro[4.5]decan-2-one;
(trans)-3-(6-fluoro-2-pyridinyl)-8-{5-[(trifluoromethyl)oxy]-1H-benzimidazol-2-yl}-1-oxa-3-azaspiro[4.5]decan-2-one;
(cis)-3-(6-fluoro-2-pyridinyl)-8-{5-[(trifluoromethyl)oxy]-1H-benzimidazol-2-yl}-1-oxa-3-azaspiro[4.5]decan-2-one;
3-(6-fluoro-2-pyridinyl)-8-[5-(trifluoromethyl)-1H-benzimidazol-2-yl]-1-oxa-3-azaspiro[4.5]decan-2-one;
3-(2-pyridinyl)-8-[6-(trifluoromethyl)-1H-imidazo[4,5-c]pyridin-2-yl]-1-oxa-3-azaspiro[4.5]decan-2-one;
and 3-(2-pyridinyl)-8-[6-(trifluoromethyl)-1H-imidazo[4,5-b]pyridin-2-yl]-1-oxa-3-azaspiro[4.5]decan-2-one.

5. A method of treating a condition for which modulation of NPY-Y5 receptors is beneficial, which comprises administering to a mammal (e.g. human) in need thereof an effective amount of a compound of claim 1.

6. A method as claimed in claim 5, wherein the condition is eating disorders.

7. A method as claimed in claim 6, wherein the condition is binge eating.

8. A method as claimed in claim 6, wherein the condition is obesity.

9. A method as claimed in claim 5, wherein the condition is depression.

10.-20. (canceled)

21. A pharmaceutical composition comprising a compound as claimed in claim 1 and a pharmaceutically acceptable carrier.
